SEQUENCE LISTING (34 sequences)

SEQ ID NO: 1 - protein, 289 aa, Paenibacillus sp.
MNNRENEDILIGQIKYSNVWPVFHYFDVTRLSQVSQLVTEVPSKLNRRMLGGMLDISPVSSFAYGYGSERFLLLPDLSVSSDGPVGSILLFSRKPPEEIANGKIALTNTSATSVNLLKIIMEKAYGGKPEYWDSEPDLDLMMAESDGALLIGDHAIQASWRDHDYIVTDLGEVWKSWTGYGMTFAVWAVQKSFAEAHGDYLAEVTEAFVESKRKSLSDVSPLVAKACAEIGGTADYWRHYFTQNIRYDFNESHRQGLSLYFDYAYEMGLLDHRVELEIWSDNLLTRVKE

SEQ ID NO: 2 - protein, 286 aa, Paenibacillus sp.
MTGRLNHPLRVGRIDYANAWPLFHYLEDYTEQAGIDIVLKVPAELNRLLREGEIQVSAVSSFAYGMNDKDYVLLPHLSVGSEGKVNSILLFLKQPIEQSSPERIAVTTTSATSVNLLKILMKKYYELDPAYESAEPDLDRMLEWADGALLIGDPAIRESWENKGLTVIDLGEVWNQWTGLAMTYAVVAARKEAVEQSPEAFERLFQAFTASKEKSVGDLKPLIQKAMEQLGGDPDYWQMYFTCLNYDFGPKQQEGLTLYFRYASELGLLDHDVNLHFYKEESLPNR

SEQ ID NO: 3 - protein, 241 aa, Pedobacter heparinus
MNKIKISAVSYTNTKPFIYGIEHSALLDQIDLSLDIPTDCAAKLIDGQVDIGLIPVAAIPDVPNAHIVADYCIGSVGAVNSVFIFSKVPVAEIGTVKLDLHSRTSNHLAKVLLKFHFKQQVSYTTDQAAETDAIVLIGDRTFGRKEEFAYAYDMGQEWMNFTGLPFMYAAWVANKPIPQAFINDFNEALAFGLSKRKALLQELPEQANFDLEDYLLHKLDFELTAKKREALELFLMYITKL

SEQ ID NO: 4 - protein, 365 aa, Corynebacterium glutamicum
MSAVQIFNTVHVNGSSPYDVHIGSGLNELIVQRAAESGAEQVAILHQPSMDDIASELDAALVAAGLKVLHLNVPDAENGKSLEVAGQCWDELGGAAFGRRDIVIGLGGGAATDLAGFVAAAWMRGVRVIQVPTTLLAMVDAAVGGKTGINTAAGKNLVGAFHEPDAVFIDTDRLATLPDAEIIAGSAEIIKTGFIADPEILRLYETDPAACLKKEVEGSHLPELIWRSVTVKGSVVGQDLKESSLREILNYGHTFAHAVELRENFRWRHGNAVAVGMMFIANLSHKLGLIDAPLLERHRSILAAIGLPTSYEGGAFDELYDGMTRDKKNRDGNIRFVALTAVGEVTRIEGPSKQDLQSAYEAISH

SEQ ID NO: 5 - protein, 169 aa, Corynebacterium glutamicum
MLLPIVVLVGLPGAGKSTIGRRLARALNTELVDSDELIERATGKACGAVFSELGEPAFRELEAIHVAEALKSSGVVSLGGGSVLTESTRELLKGQDVVWIDVPVEEGIRRTANERSRPVLQAADPAEHYRNLVKVRTPLYEEVATYRLRTNNRSPQQVVAAVLHHLEID

SEQ ID NO: 6 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 7 - protein, 292 aa, Streptomyces griseus
MDNSTVSPREGDDRRSRPRVGHIQFLNCLPLYWGLARTGTLLDLELSKDTPERLSEQLIRGDLDIGPVTLVEYLRNADDLVAFPDIAVGCDGPVMSCVIVSQLPLEQLDRARVALGSTSRTSVRLAQLLLAERYGVRPDYYTCPPDLGLMMQEADAAVLIGDAALRANLHDAPRLGLQVHDLGRMWKEWTGLPFVFAVWAARKDFLAAHPETAAKVHEAFLASRDLSLEEVAKVAEQAARWEAFDAEVLERYFTTLDFRFGPDQLAGVREFARRTGRDTGYPADVHVELLGG

SEQ ID NO: 8 - protein, 282 aa, Streptomyces coelicolor
MDNSRTRPRVGHIQFLNCLPLYWGLARTGTLLDFELTKDTPEKLSEQLVRGDLDIGPVTLVEFLKNADDLVAFPDIAVGCDGPVMSCVIVSQVPLDRLDGARVALGSTSRTSVRLAQLLLSERFGVQPDYYTCPPDLSLMMQEADAAVLIGDAALRANMIDGPRYGLDVHDLGALWKEWTGLPFVFAVWAARRDYAEREPVITRKVHEAFLASRNLSLEEVEKVAEQAARWEAFDEDTLAKYFTTLDFRFGAPQLEAVTEFARRVGPTTGFPADVKVELLKP

SEQ ID NO: 9 - protein, 287 aa, Streptomyces sp.
MDVYRSRPRVGHIQFLNCLPLYWGLARTGTLLDLELTKDTPEKLSERLVRGELDIAPITLVEFLRNADQLVAFPDLAVGCDGPVMSCVIVSQVPLEQLDGARVALGSTSRTSVRLAQLLLAEQYGVRPDYYTCPPDLGLMMQEADAAVLIGDAALRASLHDAPRLGLAVHDLGQMWKEWTGLPFVFAVWAARKDYLAREPVVVRKVHEAFLASRDVSLEEVTKVAEQAARWEAFDAELLERYFTTLDFRFGPEQLAGVREFARRTGATTGYPADIAVELLAAVEASH

SEQ ID NO: 10 - protein, 282 aa, Streptomyces collinus
MDNSRTRPRVGHIQFLNCLPLYWGLARTGTLLDFELTKDTPEKLSEQLVRGDLDIGPVTLVEFLRNADDLVAFPDIAVGCDGDVMSCVIVSQVPLDRLDGARVALGSTSRTSVRLAQLLLAERFGVRPDYYTCPPDLSLMMQESDAAVLIGDAALRANLIDGPRYGLEVHDLGALWKEWTGLPFVFAVWAARRDYAEREPFITRKVHEAFLASRNLSLEEVGKVAEQAARWESFDEATLEQYFTTLDFSFGGPQLKAVAEFARRVGPTTGFPADVRVDLLQP

SEQ ID NO: 11 - protein, 283 aa, Salinispora arenicola
MVDPVARPRVGHIQFLNCLPIYWGLMRSGALIDVDLRKDSPDRLSAALVAGDLDIGPITLVEYLRNTDDLLLLPDLAVGSDGPVLSVNIVSTRPLADLDRRRVALGSTSRTGVLLAQLLLAERYGVRPDYFRCPPDLTQMLLEADAGVLIGDVALRALLEAPSRGLTVTDLGQAWHEWTGLPMVFAVWAVRRDFAAAHPGLVKEVHEAFLRSRDLCLAELDQVAAAAARWESFDADTLATYFRTLDFSLGDRQIAGLREFARRAAAADATPELPAGGPARFIG

SEQ ID NO: 12 - protein, 282 aa, Streptomyces leeuwenhoekii
MDNSRTRPRVGHIQFLNCLPLYWGLARTGTLLDFELTKDTPEKLSEQLVRGDLDIGPITLVEFLKHADRLVAFPDIAVGCDGPVMSCVIVSQVPLERLDGARVALGSTSRTSVRLARLLLEERIGVRPDYYTCPPDLGLMMREAEAAVLIGDAALRANMLDGPRYGLDVHDLGALWKEWTGLPFVFAVWAARRDYLEREPVITRKVHEAFLASRNLSLQEVDKVAEQAARWEAFDEGTLARYFKTLDFRFGAPQLEAVAEFARRVGPTTGFPADVKVELLQP

SEQ ID NO: 13 - protein, 249 aa, Leptospira sp.
MKIGIVKHLNARPLTWGFEKNKEHVVVYENPAILKDYLLNGEIDLGLISSIECVRNQDALNTYYGTGVCAQEKVRSILFFQNKKEKFPPELILTDNGSKTSVALVRVLVHEKCGFVPDVEPTDSNFIRERITKGIGSHMLFGDNALLAKWDPKFYEVKDLAQWWNEITGLGFIFALWAGKKSLKVDDSIFIQSLEYGISHIEEIISYESRLSSTLVREYLTKELHYKITEKDRKGFLLFSEKCRKLDLL

SEQ ID NO: 14 - protein, 356 aa, Escherichia coli
MQKDALNNVHITDEQVLMTPEQLKAAFPLSLQQEAQIADSRKSISDIIAGRDPRLLVVCGPCSIHDPETALEYARRFKALAAEVSDSLYLVMRVYFEKPRTTVGWKGLINDPHMDGSFDVEAGLQIARKLLLELVNMGLPLATEALDPNSPQYLGDLFSWSAIGARTTESQTHREMASGLSMPVGFKNGTDGSLATAINAMRAAAQPHRFVGINQAGQVALLQTQGNPDGHVILRGGKAPNYSPADVAQCEKEMEQAGLRPSLMVDCSHGNSNKDYRRQPAVAESVVAQIKDGNRSIIGLMIESNIHEGNQSSEQPRSEMKYGVSVTDACISWEMTDALLREIHQDLNGQLTARVA

SEQ ID NO: 15 - protein, 350 aa, Escherichia coli
MNYQNDDLRIKEIKELLPPVALLEKFPATENAANTVAHARKAIHKILKGNDDRLLVVIGPCSIHDPVAAKEYATRLLALREELKDELEIVMRVYFEKPRTTVGWKGLINDPHMDNSFQINDGLRIARKLLLDINDSGLPAAGEFLDMITPQYLADLMSWGAIGARTTESQVHRELASGLSCPVGFKNGTDGTIKVAIDAINAAGAPHCFLSVTKWGHSAIVNTSGNGDCHIILRGGKEPNYSAKHVAEVKEGLNKAGLPAQVMIDFSHANSSKQFKKQMDVCADVCQQIAGGEKAIIGVMVESHLVEGNQSLESGEPLAYGKSITDACIGWEDTDALLRQLANAVKARRG

SEQ ID NO: 16 - protein, 301 aa, Streptomyces caniferus
MSEPLLTASAAPAPRSGRRPRVGHIDFLNCLPLFWGLARSGSLLDLDLRKGSPDALSDELVAGGLDIGPISLLEFLKHADDLVALPDIAVGSDGPVESCLIVSQVPLDQLDGEPVALGSTSRTSVRLAELLFAESVGVRPEYFVCPPDLAAMMKQARAAVVIGDAALRASLHEAPRLGLHVHDLGGMWRDLTGLPFVFAVFAARKDFLAKDPETVHRVHADLLASRDLSLAAVKEVCEQAARWEAFEARTLEHYYTTALDFSLGERQLSGIAEFARRVGGGQAGFPPDVRVQLLGSEAISM

SEQ ID NO: 17 - protein, 294 aa, Desulfovibrio vulgaris
MRRAPRLTPCGERTMHTPCTYPLRLGRIGYLNVLPIYHPLETGIIPHEFDIVAGPPAVLNNMMARGDLHVSATSSIEYGRHADDYLLVPDMAIGSRGPVMSVLLLSRRPVEELAGQTVLVSAETHTSAALLRLLFREYYRFPVSYVTGCATTQIAGPEAPEAFLAIGDEALRLRNHPDYPHVLDLGEAWRQWTGLPFIFGVWVVSRAAAAACRFSENPADLLRRARDWGEAHMDTILDLTLQGCPLTREELRVYYDGLVYHLGDEEQEGLRLFYSRLAASGDLARAPALAFFGA

SEQ ID NO: 18 - DNA, 867 bp, Paenibacillus sp.
atgaacaaccgtgaaaacgaagatatcctgatcggccagatcaagtactccaacgtgtggccagtgttccactacttcgatgtgacccgtctgtcccaggtgtcccagctggtgaccgaagtgccatccaagctgaaccgtcgtatgcttggcggtatgctggatatctccccagtgtcctccttcgcatacggctacggctccgaacgtttcctgctgctgccagatctgtccgtgtcctccgatggtccagtgggctccatcctgctgttctcccgtaagccaccagaagaaatcgcaaacggcaagatcgcactgaccaacacctccgcaacctccgtgaacctgctgaagatcatcatggaaaaggcatacggtggcaagccagaatactgggattccgaaccagatttggatctgatgatggcagaatccgatggcgcactgctgatcggcgatcacgcaatccaggcatcctggcgtgaccacgattacatcgtgaccgatctgggcgaagtgtggaagtcctggaccggctacggtatgaccttcgcagtgtgggcagtgcagaagtccttcgcagaagcacacggcgattacctggcagaagtgaccgaggcattcgtggaatccaagcgtaagtccctgtccgatgtgtccccactggtggcaaaggcatgcgcagaaatcggtggcaccgcagattactggcgtcactacttcacccagaacatccgttacgatttcaacgaatcccaccgtcagggcctgtccttgtacttcgattacgcatacgaaatgggcctgctggatcaccgtgtggaactggaaatctggtccgataacctgttgacccgtgtgaaagag

SEQ ID NO: 19 - DNA, 858 bp, Paenibacillus sp.
atgaccggtcgtctgaatcacccactgcgtgttggccgtatcgattacgcaaacgcatggccactgttccactacctggaagattacaccgaacaggcaggcatcgatatcgtgctgaaggtgccagcagaactgaaccgtctgctgcgtgaaggcgaaatccaggtgtccgcagtgtcctccttcgcatacggcatgaacgataaggattacgtgctgctgccacacctgtccgtgggctccgaaggcaaggtgaactccatcctgctgttcttgaagcagccaatcgaacagtcctctccagaacgtatcgcagtgaccaccacctccgcaacctccgtgaacctgctgaagatcctgatgaagaagtactacgaattggatccagcatacgaatccgcagaaccagatttggatcgtatgttggaatgggcagatggcgcactgctgatcggcgatccagcaatccgtgaatcctgggaaaacaagggcctgaccgtgatcgatctgggcgaagtgtggaaccagtggaccggcttggcaatgacctacgcagtggtggcagcccgtaaagaagcagttgaacagtccccagaagcattcgaacgtctgttccaggcattcaccgcatccaaagaaaagtccgtcggcgatctgaagccactgatccagaaagcaatggaacagcttggcggtgatccagattactggcagatgtactttacctgcctgaactacgatttcggtccaaagcagcaagaaggcctgaccttgtacttccgttacgcatccgaactgggcctgctggatcacgatgtgaacttgcacttctacaaagaagaatccctgccaaaccgc

SEQ ID NO: 20 - DNA, 723 bp, Pedobacter heparinus
atgaacaagatcaagatctccgcagtgtcctacaccaacaccaagccattcatctacggcatcgaacactccgcactgctggatcagatcgatttgtccctggatatcccaaccgattgcgcagcaaagctgatcgatggccaggtggatatcggcttgatcccagtggcagcaatcccagatgtgccaaacgcacacatcgtggcagattactgcatcggctccgttggcgcagtgaactccgtgttcatcttctccaaggtgccagtggccgaaatcggcaccgtgaagctggatctgcactcccgtacctccaaccacctggcaaaggtgctgctgaagttccacttcaagcaacaggtgtcctataccaccgatcaggcagcagaaaccgatgcaatcgtgctgatcggcgatcgtaccttcggtcgtaaagaagaattcgcatacgcatacgatatgggccaagaatggatgaacttcaccggcttgcctttcatgtacgcagcatgggtcgcaaacaagccaattccacaggcattcatcaacgatttcaacgaagcactggcattcggcctgtccaagcgtaaggcactgctgcaagaactgccagaacaggcaaacttcgatctggaagattacctgctgcacaagctggatttcgaactgaccgcaaagaagcgtgaagccctggaactgttcctgatgtacattaccaagctg

SEQ ID NO: 21 - DNA, 1095 bp, Corynebacterium glutamicum
atgagcgcagttcagatctttaacactgtgcacgtgaacggctcttccccctacgatgtgcacatcggttccggtttgaacgaactcattgtccaacgcgccgctgagagcggcgccgaacaggttgcgatcctgcatcagccatctatggatgatatcgcgtctgagctggatgcagcccttgtcgcagctggcttgaaggtgctgcacttgaacgttcctgatgcggagaacggtaagtcgctggaggtggcgggccaatgctgggacgaactgggtggtgcagctttcggacgtcgtgacatcgtgattggtctgggaggcggtgccgcgacagatcttgcaggctttgtggccgcggcgtggatgcgcggcgttcgtgttatccaggttccgacaacacttcttgccatggtggacgccgcagtgggcggtaaaaccggaatcaacactgcggcaggcaagaacctggttggcgcgttccacgaaccagatgcagtttttattgatacagaccgcctcgctacgctgcctgatgcagaaattattgcgggttccgcagagattattaagactggctttatcgccgatccagaaattctgcgactctacgaaaccgaccctgccgcatgccttaagaaggaggtggagggctcccatctgcctgaacttatctggcgatctgtaaccgtcaagggtagcgtcgtgggtcaggatcttaaggagtcgtccctccgagaaattttgaactatggccacaccttcgcccatgcggtcgaacttcgagaaaattttcgctggcgccacggcaacgctgtcgccgttggcatgatgttcattgccaatctgtcgcacaagcttggcctcatcgatgccccactgctggagcgccatcgctcaatcttggccgcgatcggactgccaactagctacgaaggcggtgcatttgacgaactctatgacggtatgacccgtgataaaaaaaaccgagacggtaatatccgtttcgtggcccttaccgcagtaggcgaagtcacccgtatcgaaggcccgtccaaacaggacctgcagagcgcgtatgaagcgatctcccac

SEQ ID NO: 22 - DNA, 507 bp, Corynebacterium glutamicum
atgcttctccccatcgttgttcttgttggccttccaggtgccggtaagtctaccatcggacgacgactggcgcgcgcactcaacacagagcttgtggattccgatgagctcatcgagcgagcaacaggcaaggcctgcggcgccgtattctcggagctcggcgagccagcattccgcgagcttgaagctattcacgtcgctgaagccttgaagtcctccggtgtggtgtccctgggtggaggcagcgtgctgactgaatcgacgcgcgaacttctgaagggccaagatgtggtttggattgatgtcccagtcgaggaaggaatccgtcgcactgctaacgaacgttctcgaccagtgcttcaagccgcagatcctgctgagcactatcgcaatctggtaaaggtccgcacccctctctatgaagaagtggcaacctatcgtctgcgaacaaacaatcgctccccacagcaagtagtcgctgcggtgctccaccacttggagatcgat

SEQ ID NO: 23 - DNA, 1110 bp, Saccharomyces cerevisiae
atgtcggaaagcccaatgttcgccgccaacggcatgccaaaagttaatcagggcgccgaagaggacgtccgcattttgggctatgatcccctcgcctcccccgcactcctgcaagtacagattcccgccaccccgacctctttggaaaccgcgaagcgcggccgccgagaagcgattgatattatcaccggcaaggatgaccgagtactcgttatcgtcggtccctgctcgatccacgacctcgaagccgcgcaggagtatgcattgcgattgaagaagctgtctgatgagttgaagggtgatctttcgattattatgcgtgcgtacctggaaaagcctcgcaccaccgtgggttggaagggattgatcaatgatcctgacgtaaacaacaccttcaacatcaataagggcctccaatctgcccgccagcttttcgttaacctcactaacattggccttcccatcggctccgaaatgctcgatacgatctcgccgaaatacctggccgatttggtgtcctttggtgcaattggtgcccgtaccaccgaatcacaactgcaccgcgaattggcaagcggattgagcttccccgtgggtttcaaaaacggtactgatggcactcttaacgtggccgtcgatgcatgtcaagccgccgcacactcccaccatttcatgggtgtgactaaacacggagtggcggcgattactaccaccaagggtaatgagcactgcttcgtcatcctgcgtggcggaaagaagggtaccaactacgacgcaaagagcgtcgctgaggccaaggcccaattgccagcaggttccaatggccttatgatcgattattcccacggcaattctaataaggactttcgcaaccaaccgaaggtgaatgatgtggtatgtgagcagatcgcgaatggtgaaaatgcgatcactggcgtgatgatcgaatcgaatattaatgagggcaaccagggaattcctgcggagggaaaggcgggtttgaagtacggcgtatctattactgacgcttgcattggctgggagaccaccgaggacgtgctgcgcaagcttgcagcggcggtccgccagcgtcgtgaagttaataaaaaa

SEQ ID NO: 24 - DNA, 876 bp, Streptomyces griseus
atggataattcaaccgtgagccctcgtgaaggtgatgaccgtcgttctcgtccacgcgtgggccatattcagtttctgaactgtcttcccctctactggggacttgcacgaactggaacacttcttgatttggagctcagcaaagacacgccagagcgtctgtccgagcagttgatccgcggcgatctggatatcggaccagttacccttgttgagtatctccgtaatgcagacgaccttgtggcattcccagatatcgcggttggctgcgacggacccgtgatgtcttgtgtaatcgtgtcgcaacttccgctggaacagcttgatcgcgcccgtgtggcgctcggatcaacctcacgcacgtcagttcgtctggcgcagctgctgcttgctgaacgctacggcgttcgccccgactactatacatgcccacctgatctgggtctcatgatgcaagaagcagacgctgctgtgctcatcggtgatgcggcactgcgtgcgaacttgcatgatgcaccccgcctcggcttgcaggttcacgatctgggacgcatgtggaaggaatggaccggccttcctttcgtattcgcagtttgggccgcccgcaaggattttctggctgcacacccggaaacagctgccaaagttcacgaagcgtttttggcctctcgtgatctcagccttgaggaggtcgcaaaggttgctgaacaagctgcccgctgggaggcctttgacgccgaggtattggaacgttactttactacgttggactttcgtttcggacccgaccagctcgctggagttcgcgaatttgcccgtcgtaccggtcgcgatacgggttatccagcagacgtgcacgtggagctgctgggcggc

SEQ ID NO: 25 - DNA, 846 bp, Streptomyces coelicolor
atggacaactcccgcacgcgcccacgcgtgggtcatatccaatttctcaactgccttcccctttattggggcttggcccgcacaggcacccttctcgacttcgagctgaccaaagatacaccagaaaaactttcggagcaactggttcgtggagatttggatatcggccccgtgaccctggttgagtttttgaaaaacgcagatgatcttgttgccttcccggacatcgcggtcggctgcgatggacctgttatgtcgtgtgtaattgtctcgcaggtgcctctggatcgcctcgatggtgcacgagtcgcactcggaagcacatcccgtacttctgtgcgtctcgctcagctcctcctgtctgagcgcttcggtgtccaaccggattactacacctgccctcccgatttgtccctgatgatgcaggaagcagacgccgcggtgttgatcggcgacgcggcacttcgcgcaaacatgattgatggcccccgatacggactcgacgtgcacgacctcggagcattgtggaaagagtggacgggcctcccattcgttttcgccgtttgggctgcccgccgagactacgcagaacgcgagccagtgatcacccgtaaggtccacgaggcctttcttgccagccgtaacctttcacttgaagaggtggagaaagtggccgagcaagctgcgcgctgggaggcattcgacgaggataccctggctaaatactttactactttggatttccgctttggcgcaccacagcttgaagccgtgacggagtttgcgcgtcgtgttggcccgaccactggtttccccgcagacgttaaagtcgaactgcttaaaccg

SEQ ID NO: 26 - DNA, 861 bp, Streptomyces sp.
atggacgtgtaccgctcgcgacctcgagtcggccatatccaatttctgaattgccttcccctctattggggccttgcgcgtacgggaaccttgcttgatctcgagctgactaaggataccccagagaagcttagcgagcgtctggtccgtggtgagttggacatcgcgcctatcaccctggtggaattcctgcgtaacgcagatcagttggttgcttttccggatcttgcggtgggttgcgacggacccgtgatgtcttgcgttatcgtttctcaagtgccgctggagcaacttgacggcgcccgagttgcactgggttcaaccagccgcacgtccgtacgtcttgcccaacttctccttgctgaacagtatggtgtccgtcctgactattacacctgcccacctgatcttggtttgatgatgcaggaggcggatgcagccgtgttgatcggcgatgcagctctgcgtgcttccctccacgatgctccacgacttggcctggctgtccatgacttgggccagatgtggaaagaatggacgggcctcccattcgtattcgctgtctgggctgcgcgcaaagattacctggcacgcgagcctgtcgtggtccgtaaggttcacgaggccttcctcgcatcacgtgatgtctctctggaagaggttaccaaggtggcagaacaggccgctcgctgggaagcttttgacgcggagctgctggaacgctacttcactaccctcgacttccgctttggcccggaacaactcgcaggtgttcgcgaatttgcgcgtcgcaccggtgcaaccaccggttatcccgcagatattgccgttgaacttctggcagcggtggaggcctcgcat

SEQ ID NO: 27 - DNA, 846 bp, Streptomyces collinus
atggataatagccgcacacgcccacgcgtgggacatatccaattcttgaactgtttgcctctctactggggcctcgcgcgcaccggaactctgcttgatttcgagttgactaaggataccccagaaaagctgtccgaacagttggtgcgaggtgacctcgatattggcccagtgacccttgtggaattccttcgtaacgctgacgacctggtggcattccctgatatcgcagtcggttgcgatggtgatgtgatgagctgcgtcattgtgtctcaggtaccgctggaccgcctggacggtgcccgtgttgcgctcggcagcaccagccgcacgtccgtgcgcctggcccaactgctcctggcggaacgattcggcgtgcgtcccgactactacacttgcccacccgacctctcacttatgatgcaagaaagcgatgcagctgtactgattggcgacgcagcgctccgtgccaaccttatcgacggcccacgctatggcctggaagttcacgatcttggtgcgttgtggaaagaatggaccggacttccctttgttttcgctgtctgggctgcccgccgcgactacgctgaacgcgagcctttcatcacacgcaaggtgcacgaggccttcctcgcttcacgcaatctgtccctcgaagaagtaggaaaggtggctgagcaagcagcccgctgggaaagcttcgacgaagctacccttgagcagtatttcactacgctcgatttctcatttggaggtccccagctgaaggcggtggccgaatttgcgcgacgtgtgggccctaccaccggcttcccggctgatgtccgcgtcgatctcctgcaaccg

SEQ ID NO: 28 - DNA, 849 bp, Salinispora arenicola
atggtggatccagtggcgcgtccccgcgtgggccacattcagttcctgaactgtcttccgatttactggggactcatgcgctccggtgccctgatcgatgttgaccttcgtaaggactcccctgatcgcctgtccgccgcactcgtggcaggtgacctggacattggcccaatcacgttggtcgagtaccttcgtaacacggatgacctgttgctcctcccagaccttgcggttggttcggatggaccggttctgtcagtaaacattgtttctactcgtcctcttgcagatctggatcgtcgtcgagtcgcactcggtagcacctcgcgcaccggcgtccttctggcccagcttctgctcgcggagcgctacggcgtacgcccagattactttcgctgcccaccggatctcacccaaatgctgctcgaagcagatgctggtgttttgatcggagatgtggcgttgcgtgctctcctggaagctccatcccgcggtttgaccgtcactgacctcggtcaggcctggcacgagtggacgggtctccctatggtattcgcagtctgggcagtgcgacgcgatttcgccgccgcccacccaggcctggttaaggaagttcatgaagcctttcttcgatcccgcgacctgtgcctcgcagagctggatcaggttgccgcggctgctgcccgctgggagtctttcgacgcagatacgcttgctacgtactttcgaactctggatttttctttgggtgatcgtcagattgcaggcctccgtgagttcgctcgccgcgcagcggcagctgatgcgactcctgaactcccagcaggcggccctgcccgttttattggc

SEQ ID NO: 29 - DNA, 846 bp, Streptomyces leeuwenhoekii
atggataactctcgcacccgccctcgcgttggtcacatccaatttttgaactgcctcccactgtattggggcctggcacgtacaggtacattgcttgattttgaactgacgaaggacactcccgagaagctcagcgaacagcttgtccgtggagatctcgatattggaccaatcacccttgtagaattcctcaagcacgcagaccgccttgttgcatttcccgatattgccgtgggctgcgatggtcctgtcatgtcatgcgttattgtcagccaagtccccttggaacgtctcgatggtgcccgtgttgcgctgggatcaacgtctcgtacatccgttcgcctcgcacgccttcttcttgaggagcgcattggcgtgcgtcctgactattacacatgcccgcccgacctcggcctcatgatgcgtgaggccgaggctgccgtgctgatcggagacgcagcactccgcgcaaacatgcttgacggcccccgctatggtctcgatgtccacgaccttggtgcactgtggaaggagtggaccggtctgccttttgtgtttgcagtgtgggcagcccgacgcgactatctggagcgagaaccggtaatcacccgtaaggtccatgaagcgttcttggcgtcccgaaacctgtccctgcaagaagtggacaaggtggcagaacaagcagcacgttgggaagcatttgatgaaggaacccttgcgcgctacttcaagaccttggacttccgatttggagcaccccagttggaagctgtcgctgagtttgctcgtcgcgtgggcccgacaaccggattccctgcagacgtgaaggttgagcttttgcagcct

SEQ ID NO: 30 - DNA, 858 bp, Leptospira sp.; identical to SEQ ID NO: 19

SEQ ID NO: 31 - DNA, 1068 bp, Escherichia coli
atgcagaaggatgccctgaataaagtgcacatcaccgacgaacaggtacttatgacccctgaacagcttaaggccgcgttcccgctttccctgcagcaagaagcccaaattgcggactctcgcaaatccatctcagatatcatcgcaggtcgcgatccccgcctgttggtagtgtgcggtccctgcagcatccatgacccggagaccgcactggaatatgcgcgccgcttcaaagcccttgccgcggaggtgtcagactcattgtatcttgtgatgcgcgtgtacttcgaaaagccccgtaccacggttggatggaaaggcctgatcaacgaccctcacatggatggctccttcgatgtcgaggcaggcctgcaaatcgcccgcaagctgctcctggagttggtcaacatgggattgcccttggccacagaagcattggatcccaatagcccgcaatatctgggcgatctcttctcctggtcggctatcggcgcacgcaccaccgaatcgcagacacaccgcgaaatggcatccggtttgtccatgccagttggatttaagaacggtactgatggttcgctcgccaccgccattaacgcaatgcgcgcggccgcccagccacatcgcttcgttggcatcaatcaagcaggccaggttgctctgttgcagacccaaggcaaccctgacggccacgtgatcctccgcggtggtaaggctccaaactattcccccgcggacgtggcccagtgtgagaaggagatggaacaggccggactgcgaccctccctgatggtggattgttcccacggcaattccaacaaagactaccgtcgccagcccgccgtggctgagagcgtcgtcgctcagattaaagatggcaaccgctccatcattggattgatgattgagagcaacattcatgaaggaaaccagtcctcagaacaaccacgctcggaaatgaagtatggtgtgtctgtcaccgatgcctgcatctcctgggagatgacggacgctctgctgcgcgagatccaccaagatcttaacggccagttgaccgctcgcgtcgcc

SEQ ID NO: 32 - DNA, 1050 bp, Escherichia coli
atgaattaccagaacgacgaccttagaattaaggaaataaaagagttgctacctcccgtcgcgctgttggaaaaattccccgccacagaaaatgctgctaatacggtcgcacatgctagaaaggctattcataaaattctgaaaggtaatgatgatagattattggttgttattggtccatgttctatacacgaccccgtagccgcaaaagaatatgctaccagacttttagctttacgtgaagagttgaaagatgaacttgaaattgtcatgcgtgtgtacttcgaaaaaccgagaacgaccgttggttggaagggcttaatcaacgacccacatatggacaactctttccagatcaatgatggcctaaggatcgcacgtaaacttctattagatattaatgattcaggacttcccgctgcaggagaattccttaatatgattactcctcagtatctagccgacttaatgtcctggggtgccatcggtgcgagaacgacagaaagtcaagttcacagggaactggcctccggtttgtcctgtccagttggtttcaagaacggtacagatggtaccatcaaagtcgcaattgacgccataaacgccgccggagctccacactgctttttgagcgtcactaaatggggacactcagccatagtcaacacttcaggtaacggtgattgtcatattattcttagaggtggcaaggaacctaactactccgccaaacatgttgctgaagttaaagaaggtcttaacaaggcgggtcttccggcccaagtgatgatagattttagccatgccaacagctctaagcaatttaaaaaacaaatggatgtttgtgctgatgtttgtcaacaaatcgcgggtggggagaaagcgatcattggtgtaatggtcgaaagccacttggttgaaggcaaccaaagtttagaatctggagagcccttagcgtatggaaaaagcatcacagatgcgtgtataggttgggaagacactgacgccttactaagacaactagctaatgcagttaaggcccgtagaggg

SEQ ID NO: 33 - DNA, 903 bp, Streptomyces caniferus
atgagcgaaccactgttgaccgcatcagcagcaccagcccctcgctccggccgtcgtccacgtgtgggacacatcgatttccttaactgcctgcccctgttttggggcctggcaagatccggctccttgttggatttggatttgcgtaagggctccccagatgccctttcggacgaattggtggcaggcggcttggacatcggtcctatttctttgctggagttcctgaaacatgctgatgacttggtcgcgctgccagacatcgcagttggttccgacggcccagtggaatcctgcttgattgtgtctcaggtcccgcttgatcaactcgacggcgagcccgtcgcgttgggctctacctctcgtacctctgtgcgactggccgaattgttgttcgctgagtccgttggcgtgcgtcctgaatactttgtttgtccacctgatctggctgcgatgatgaagcaggcacgtgcagccgtggtcattggtgacgctgcgctgcgcgcgtcccttcacgaggcaccacgtttgggcctgcacgtccatgatttgggcggcatgtggcgagacttgaccggtctgccgttcgtttttgcggtgttcgcagcccgtaaggattttctggcaaaagaccccgaaactgtccaccgtgttcatgctgatttgctggcatcccgtgatttgtccttggctgcggtgaaagaagtctgtgagcaggcagcccgttgggaagcattcgaggcgcgaaccttggagcactactataccactgcattggatttctcactgggcgaacgtcaactgtctggtatcgcagagttcgcacgtcgtgtgggcggcggccaggcaggatttccgcccgacgtccgtgttcaacttctcggctctgaagcaatctccatg

SEQ ID NO: 34 - DNA, 882 bp, Desulfovibrio vulgaris
atgcgtcgagccccgcgcctgaccccatgcggcgaacgtaccatgcacaccccatgtacctacccattgcgtttgggccgaatcggttaccttaacgtcctcccgatctaccaccccttggaaaccggaatcattccgcatgagttcgacatcgttgcaggcccacctgccgtgctgaacaatatgatggcgcgtggtgacttgcacgtgtccgcaacctcttctatcgagtacggccgtcacgcggatgactatttgctggtcccagatatggcaattggctcccgtggcccagtgatgagcgttcttctcttgtcccgtcgtccagtggaagaattggccggccagactgttcttgtgtctgctgaaacccacacctctgcagccctgcttcgcttgttgttccgtgagtactatcgttttcctgtgtcctacgtcaccggctgcgctaccactcaaatcgcgggtccggaagcaccagaggctttcctggcgattggtgacgaagccctgcgccttagaaaccacccagattacccacacgtgttggatttgggcgaggcttggcgtcagtggaccggtttgccgttcatctttggagtgtgggtggtgtcccgtgcagcagcagcagcatgccgtttttctgaaaatcccgcagatctgcttcgtcgagcccgtgactggggtgaggctcacatggatactattctggaccttaccctccagggctgtccacttacccgtgaagaattgcgcgtctactatgatggcttggtttaccacttgggtgacgaagaacaggaaggcttgcgtttgttctactcccgtttggctgcatccggcgatttggcccgtgctccagcgttggcattctttggagca

(19) United States
(12) Patent Application Publication
(10) Pub. No.: US 2024/0229046 A1
(43) Pub. Date: Jul. 11, 2024

(54) ENGINEERED BIOSYNTHETIC PATHWAYS FOR PRODUCTION OF DEOXYHYDROCHORISMIC ACID BY FERMENTATION

(71) Applicant: Zymergen Inc., Emeryville, CA (US)

(72) Inventors: Cara Ann Tracewell, Walnut Creek, CA (US); Alexander Glennon Shearer, San Francisco, CA (US); Anupam Chowdhury, Emeryville, CA (US)

(21) Appl. No.: 17/925,593

(22) PCT Filed: May 18, 2021
(86) PCT No.: PCT/US2021/032980

§ 371 Date: Nov. 15, 2022

(51) Int. Cl. C12N 15/52; C12N 9/10; C12N 9/12; C12N 9/88

(52) U.S. Cl. CPC C12N 15/52; C12N 9/1085; C12N 9/1205; C12N 9/88; C12Y 205/01054; C12Y 207/01071; C12Y 402/03004

(57) ABSTRACT

The present disclosure describes the engineering of microbial cells for fermentative production of deoxyhydrochorismic acid and provides novel engineered microbial cells and cultures, as well as related deoxyhydrochorismic acid production methods.

### STATEMENT AS TO RIGHTS TO INVENTIONS MADE UNDER FEDERALLY SPONSORED RESEARCH AND DEVELOPMENT

This invention was made with Government support under Agreement No. HR0011-15-9-0014, awarded by DARPA. The Government has certain rights in the invention.

### INCORPORATION BY REFERENCE OF THE SEQUENCE LISTING

This application includes a sequence listing which has been submitted electronically in ASCII format and is hereby incorporated by reference in its entirety. This ASCII copy, created on Apr. 12, 2021, is named ZMGNPO10WO_SL.txt and is 65,560 bytes in size.

### FIELD OF THE DISCLOSURE

The present disclosure relates generally to the area of engineering microbes for production of deoxyhydrochorismic acid by fermentation.

### BACKGROUND

Deoxyhydrochorismic acid, also known as 3-((1-carboxyvinyl)oxy)benzoate, exists in nature as an intermediate in the biosynthesis of menaquinone, or vitamin K2.

The metabolic pathway to deoxyhydrochorismic acid is derived from the shikimate pathway metabolite, chorismate. Production of deoxyhydrochorismic acid by fermentation of a simple carbon source entails linking the flux of the shikimate biosynthesis pathway to a highly active chorismate dehydratase in a suitable industrial microbial host and optionally improving flux through this pathway.

### SUMMARY

The disclosure provides engineered microbial cells, cultures of the microbial cells, and methods for producing deoxyhydrochorismic acid, including the following:

Various embodiments cnetemplated herein may include, but need not be limited to, one or more of the following:

Embodiment 1: An engineered microbial cell that expresses a non-native chorismate dehydratase, wherein the engineered microbial cell produces deoxyhydrochorismic acid.

Embodiment 2: The engineered microbial cell of embodiment 1, wherein the engineered microbial cell includes increased activity of one or more upstream deoxyhydrochorismic acid pathway enzyme(s), said increased activity being increased relative to a control cell.

Embodiment 3: The engineered microbial cell of embodiment 2, wherein the one or more upstream deoxyhydrochorismic acid pathway enzyme(s) are selected from the group consisting of a glucokinase, a transketolase, a transaldolase, phospho-2-dehydro-3-deoxyheptonate aldolase, a 3-deoxy-D-arabino-heptulosonate-7-phosphate (DAHP) synthase, a 3-dehydroquinate synthase, a 3-dehydroquinate dehydratase, a shikimate dehydrogenase, a shikimate kinase, a 3-phosphoshikimate 1-carboxyvinyltransferase, and a chorismate synthase.

Embodiment 4: The engineered microbial cell of any one of embodiments 1-3, wherein the engineered microbial cell includes reduced activity of one or more enzyme(s) that consume one or more deoxyhydrochorismic acid pathway precursors, said reduced activity being reduced relative to a control cell.

Embodiment 5: The engineered microbial cell of embodiment 4, wherein the one or more enzyme(s) that consume one or more deoxyhydrochorismic acid pathway precursors are selected from the group consisting of dihydroxyacetone phosphatase and phosphoenolpyruvate phosphotransferase.

Embodiment 6: The engineered microbial cell of embodiment 4 or embodiment 5, wherein the reduced activity is achieved by replacing a native promoter of a gene for said one or more enzymes with a less active promoter.

Embodiment 7: The engineered microbial cell of any one of embodiments 1-6, wherein the engineered microbial cell additionally expresses a feedback-deregulated DAHP synthase.

Embodiment 8: The engineered microbial cell of any one of embodiments 1-7, wherein the engineered microbial cell includes increased activity of one or more enzyme(s) that increase the supply of the reduced form of nicotinamide adenine dinucleotide phosphate (NADPH), said increased activity being increased relative to a control cell.

Embodiment 9: The engineered microbial cell of embodiment 8, wherein the one or more enzyme(s) that increase the supply of the reduced form of NADPH are selected from the group consisting of pentose phosphate pathway enzymes, NADP+-dependent glyceraldehyde 3-phosphate dehydrogenase (GAPDH), and NADP+-dependent glutamate dehydrogenase.

Embodiment 10: An engineered microbial cell, wherein the engineered microbial cell includes means for expressing a non-native chorismate dehydratase, wherein the engineered microbial cell produces deoxyhydrochorismic acid.

Embodiment 11: The engineered microbial cell of embodiment 10, wherein the engineered microbial cell includes means for increasing the activity of one or more upstream deoxyhydrochorismic acid pathway enzyme(s), said increased activity being increased relative to a control cell.

Embodiment 12: The engineered microbial cell of embodiment 1-11, wherein the one or more upstream deoxyhydrochorismic acid pathway enzyme(s) are selected from the group consisting of a glucokinase, a transketolase, a transaldolase, phospho-2-dehydro-3-deoxyheptonate aldolase, a 3-deoxy-D-arabino-heptulosonate-7-phosphate (DAHP) synthase, a 3-dehydroquinate synthase, a 3-dehydroquinate dehydratase, a shikimate dehydrogenase, a shikimate kinase, a 3-phosphoshikimate 1-carboxyvinyltransferase, and a chorismate synthase.

Embodiment 13: The engineered microbial cell of any one of embodiments 10-12, wherein the engineered microbial cell includes means for reducing the activity of one or more enzyme(s) that consume one or more deoxyhydrochorismic acid pathway precursors, said reduced activity being reduced relative to a control cell.

Embodiment 14: The engineered microbial cell of embodiment 13, wherein the one or more enzyme(s) that consume one or more deoxyhydrochorismic acid pathway precursors selected from the group consisting of dihydroxyacetone phosphatase and phosphoenolpyruvate phosphotransferase.

Embodiment 15: The engineered microbial cell of embodiment 13 or embodiment 14, wherein the reduced activity is achieved by means for replacing a native promoter of a gene for said one or more enzymes with a less active promoter.

Embodiment 16: The engineered microbial cell of any one of embodiments 10-15, wherein the engineered microbial cell additionally includes means for expressing a feedback-deregulated DAHP synthase.

Embodiment 17: The engineered microbial cell of any one of embodiments 10-16, wherein the engineered microbial cell includes means for increasing the activity of one or more enzyme(s) that increase the supply of the reduced form of nicotinamide adenine dinucleotide phosphate (NADPH), said increased activity being increased relative to a control cell.

Embodiment 18: The engineered microbial cell of embodiment 17, wherein the one or more enzyme(s) that increase the supply of the reduced form of NADPH are selected from the group consisting of pentose phosphate pathway enzymes, NADP+-dependent glyceraldehyde 3-phosphate dehydrogenase (GAPDH), and NADP+-dependent glutamate dehydrogenase.

Embodiment 19: The engineered microbial cell of any one of embodiments 1-16, wherein the engineered microbial cell includes a fungal cell.

Embodiment 20: The engineered microbial cell of embodiment 19, wherein the engineered microbial cell includes a yeast cell.

Embodiment 21: The engineered microbial cell of embodiment 20, wherein the yeast cell is a cell of the genus Saccharomyces.

Embodiment 22: The engineered microbial cell of embodiment 21, wherein the yeast cell is a cell of the species cerevisiae.

Embodiment 23: The engineered microbial cell of any one of embodiments 1-22, wherein the non-native chorismate dehydratase includes a chorismate dehydratase having at least 70% amino acid sequence identity with a chorismate dehydratase from an organism selected from the group consisting of Paenibacillus sp. oral taxon 786 str. D14, Paenibacillus sp. (strain JDR-2), and Pedobacter heparinus, wherein: the chorismate dehydratase from Paenibacillus sp. oral taxon 786 str. D14 includes SEQ ID NO:1; the chorismate dehydratase from Paenibacillus sp. (strain JDR-2) includes SEQ ID NO:2; and the chorismate dehydratase from Pedobacter heparinus includes SEQ ID NO:3.

Embodiment 24: The engineered microbial cell of embodiment 23, wherein the non-native chorismate dehydratase includes a chorismate dehydratase having at least 70% amino acid sequence identity with the chorismate dehydratase from Paenibacillus sp. oral taxon 786 str. D14.

Embodiment 25: The engineered microbial cell of any one of embodiments 1 and 20-24, wherein the engineered microbial cell includes increased activity of one or more upstream deoxyhydrochorismic acid pathway enzyme(s), said increased activity being increased relative to a control cell, wherein the one or more upstream deoxyhydrochorismic acid pathway enzyme(s) comprise a dehydroquinate synthase or a shikimate kinase.

Embodiment 26: The engineered microbial cell of embodiment 25 wherein the increased activity of the dehydroquinate synthase or shikimate kinase is achieved by heterologously expressing one or both enzyme(s).

Embodiment 27: The engineered microbial cell of embodiment 26, wherein the heterologous dehydroquinate synthase has at least 70% amino acid sequence identity with a dehydroquinate synthase from Corynebacterium glutamicum including SEQ ID NO:4.

Embodiment 28: The engineered microbial cell of embodiment 26 or embodiment 27, wherein the heterologous shikimate kinase has at least 70% amino acid sequence identity with a shikimate kinase from Corynebacterium glutamicum including SEQ ID NO:5.

Embodiment 29: The engineered microbial cell of embodiment 28, wherein the engineered microbial cell expresses an additional copy of a chorismate dehydratase having at least 70% amino acid sequence identity with the chorismate dehydratase from Paenibacillus sp. (strain JDR-2) or Pedobacter heparinus.

Embodiment 30: The engineered microbial cell of any one of embodiments 7, 16, and 20-29, wherein the feedback-deregulated DAHP synthase is a feedback-deregulated variant of a S. cerevisiae DAHP synthase that includes amino acid substitution K229L and has at least 70% amino acid sequence identity with SEQ ID NO: 6.

Embodiment 31: The engineered microbial cell of any one of embodiments 1-16, wherein the engineered microbial cell is a bacterial cell.

Embodiment 32: The engineered microbial cell of embodiment 31, wherein the bacterial cell is a cell of the genus Corynebacterium.

Embodiment 33: The engineered microbial cell of embodiment 32, wherein the bacterial cell is a cell of the species glutamicum.

Embodiment 34: The engineered microbial cell of any one of embodiments 31-33, wherein the non-native chorismate dehydratase includes a chorismate dehydratase having at least 70% amino acid sequence identity with a chorismate dehydratase from an organism selected from the group consisting of Streptomyces griseus, Streptomyces coelicolor, Streptomyces sp Mg1, Streptomyces collinus, Salinispora arenicola, Streptomyces leeuwenhoekii, Leptospira mayottensis, and Paenibacillus sp. (strain JDR-2), wherein: the chorismate dehydratase from Streptomyces griseus includes SEQ ID NO:7; the chorismate dehydratase from Streptomyces coelicolor includes SEQ ID NO:8; the chorismate dehydratase from Streptomyces sp Mg1 includes SEQ ID NO:9; the chorismate dehydratase from Streptomyces collinus includes SEQ ID NO:10; the chorismate dehydratase from Salinispora arenicola includes SEQ ID NO:11; the chorismate dehydratase from Streptomyces leeuwenhoekii includes SEQ ID NO:12; the chorismate dehydratase from Leptospira mayottensis includes SEQ ID NO:13; and the chorismate dehydratase from Paenibacillus sp. (strain JDR-2) includes SEQ ID NO:2.

Embodiment 35: The engineered microbial cell of embodiment 34, wherein the non-native chorismate dehydratase includes a chorismate dehydratase having at least 70% amino acid sequence identity with a chorismate dehydratase from Streptomyces griseus including SEQ ID NO:7.

Embodiment 36: The engineered microbial cell of embodiment 35, wherein the engineered microbial cell expresses an additional copy of the chorismate dehydratase.

Embodiment 37: The engineered microbial cell of any one of embodiments 7, 16, and 31-36, wherein the feedback-deregulated DAHP synthase is a feedback-deregulated variant of an Escherichia coli K12 DAHP synthase that includes amino acid substitution P150L and has at least 70% amino acid sequence identity with SEQ ID NO:15.

Embodiment 38: The engineered microbial cell of embodiment 36 or embodiment 37, wherein the engineered microbial cell additionally expresses: a chorismate dehydratase having at least 70% amino acid sequence identity with a chorismate dehydratase from Strepomyces caniferus including SEQ ID NO:16; a chorismate dehydratase having at least 70% amino acid sequence identity with a chorismate dehydratase from Desulfovibrio vulgaris subsp. vulgaris (strain DP4) including SEQ ID NO:17 and a chorismate dehydratase having at least 70% amino acid sequence identity with a chorismate dehydratase from Paenibacillus sp. (strain JDR-2) including SEQ ID NO:2.

Embodiment 39: The engineered microbial cell of embodiment 38, wherein the engineered microbial cell expresses at least two copies each of: a chorismate dehydratase having at least 70% amino acid sequence identity with a chorismate dehydratase from Strepomyces caniferus including SEQ ID NO:16; a chorismate dehydratase having at least 70% amino acid sequence identity with a chorismate dehydratase from Desulfovibrio vulgaris subsp. vulgaris (strain DP4) including SEQ ID NO: 17; and a chorismate dehydratase having at least 70% amino acid sequence identity with a chorismate dehydratase from Penibacillus sp. (strain JDR-2) including SEQ ID NO:2.

Embodiment 40: The engineered microbial cell of any one of embodiments 1-30, wherein, when cultured, the engineered microbial cell produces deoxyhydrochorismic acid at a level of at least 20, 50, 100, 500, 1000, or 1500 mg/L of culture medium.

Embodiment 41: The engineered microbial cell of embodiment 40, wherein, when cultured, the engineered microbial cell produces deoxyhydrochorismic acid at a level of at least 200 mg/L of culture medium.

Embodiment 42: A culture of engineered microbial cells according to any one of embodiments 1-40.

Embodiment 43: The culture of embodiment 42, wherein the substrate includes a carbon source and a nitrogen source selected from the group consisting of urea, an ammonium salt, ammonia, and any combination thereof.

Embodiment 44: The culture of embodiment 42 or embodiment 43, wherein the engineered microbial cells are present in a concentration such that the culture has an optical density at 600 nm of 10-500.

Embodiment 45: The culture of any one of embodiments 42-44, wherein the culture includes deoxyhydrochorismic acid.

Embodiment 46: The culture of any one of embodiments 42-45, wherein the culture includes deoxyhydrochorismic acid at a level at least 20 mg/L of culture medium.

Embodiment 47: A method of culturing engineered microbial cells according to any one of embodiments 1-40, the method including culturing the cells under conditions suitable for producing deoxyhydrochorismic acid.

Embodiment 48: The method of embodiment 47, wherein the method includes fed-batch culture, with an initial glucose level in the range of 1-100 g/L, followed controlled sugar feeding.

Embodiment 49: The method of embodiment 47 or embodiment 48, wherein the fermentation substrate includes glucose and a nitrogen source selected from the group consisting of urea, an ammonium salt, ammonia, and any combination thereof.

Embodiment 50: The method of any one of embodiments 47-49, wherein the culture is pH-controlled during culturing.

Embodiment 51: The method of any one of embodiments 47-50, wherein the culture is aerated during culturing.

Embodiment 52: The method of any one of embodiments 47-51, wherein the engineered microbial cells produce deoxyhydrochorismic acid at a level at least 20, 50, 100, 500, 1000, or 1500 mg/L of culture medium.

Embodiment 53: The method of any one of embodiments 47-52, wherein the method additionally includes recovering deoxyhydrochorismic acid from the culture.

Embodiment 54: A method for preparing deoxyhydrochorismic acid using microbial cells engineered to produce deoxyhydrochorismic acid, the method including: (a) expressing a non-native chorismate dehydratase in microbial cells; (b) cultivating the microbial cells in a suitable culture medium under conditions that permit the microbial cells to produce deoxyhydrochorismic acid, wherein the deoxyhydrochorismic acid is released into the culture medium; and (c) isolating deoxyhydrochorismic acid from the culture medium.

### BRIEF DESCRIPTION OF THE DRAWINGS

FIG. 1: Pathway for production of deoxyhydrochorismic acid by fermentation.

FIG. 2: Deoxyhydrochorismic acid titers measured in the extracellular broth following fermentation by first-round-engineered host Corynebacterium glutamicum.

FIG. 3: Deoxyhydrochorismic acid titers measured in the extracellular broth following fermentation by first-round engineered host Saccharomyces cerevisiae.

FIG. 4: Deoxyhydrochorismic acid titers measured in the extracellular broth following fermentation by second-round engineered host C. glutamicum.

FIG. 5: Deoxyhydrochorismic acid titers measured in the extracellular broth following fermentation by second-round engineered host S. cerevisiae.

FIG. 6: Deoxyhydrochorismic acid titers measured in the extracellular broth following fermentation by third-round engineered host C. glutamicum.

FIG. 7: Deoxyhydrochorismic acid titers measured in the extracellular broth following fermentation by third-round engineered host S. cerevisiae.

FIG. 8: Deoxyhydrochorismic acid titers measured in the extracellular broth following fermentation by fourth-round engineered host C. glutamicum.

FIG. 9: Deoxyhydrochorismic acid titers measured in the extracellular broth following fermentation by fifth-round engineered host C. glutamicum.

FIG. 10: A “split-marker, double-crossover” genomic integration strategy, which was developed to engineer S. cerevisiae strains. Two plasmids with complementary 5′ and 3′ homology arms and overlapping halves of a URA3 selectable marker (direct repeats shown by the hashed bars) were digested with meganucleases and transformed as linear fragments. A triple-crossover event integrated the desired heterologous genes into the targeted locus and re-constituted the full URA3 gene. Colonies derived from this integration event were assayed using two 3-primer reactions to confirm both the 5′ and 3′ junctions (UF/IF/wt-R and DR/IF/wt-F).

FIG. 11: A “loop-in, single-crossover” genomic integration strategy, which was developed to engineer C. glutamicum strains. Loop-in only constructs (shown under the heading “Loop-in”) contained a single 2-kb homology arm (denoted as “integration locus”), a positive selection marker (denoted as “Marker”)), and gene(s) of interest (denoted as “promoter-gene-terminator”). A single crossover event integrated the plasmid into the C. glutamicum chromosome. Integration events are stably maintained in the genome by growth in the presence of antibiotic (e.g., 25 ug/ml kanamycin). Correct genomic integration in colonies derived from loop-in integration were confirmed by colony PCR with UF/IR and DR/IF PCR primers. Loop-in, loop-out constructs (shown under the heading “Loop-in, loop-out) contained two 2-kb homology arms (5′ and 3′ arms), gene(s) of interest (arrows), a positive selection marker (denoted “Marker”), and a counter-selection marker. Similar to “loop-in” only constructs, a single crossover event integrated the plasmid into the chromosome of C. glutamicum. Note: only one of two possible integrations is shown here. Correct genomic integration was confirmed by colony PCR and counter-selection was applied so that the plasmid backbone and counter-selection marker could be excised. This results in one of two possibilities: reversion to wild-type or the desired pathway integration. Again, correct genomic loop-out is confirmed by colony PCR. (Abbreviations: Primers: UF=upstream forward, DR=downstream reverse, IR=internal reverse, IF=internal forward.) See Example 1.

### DETAILED DESCRIPTION

The present disclosure describes the engineering of microbial cells for fermentative production of deoxyhydrochorismic acid and provides novel engineered microbial cells and cultures, as well as related deoxyhydrochorismic acid production methods.

### Definitions

Terms used in the claims and specification are defined as set forth below unless otherwise specified.

The term “fermentation” is used herein to refer to a process whereby a microbial cell converts one or more substrate(s) into a desired product (such as deoxyhydrochorismic acid) by means of one or more biological conversion steps, without the need for any chemical conversion step.

The term “engineered” is used herein, with reference to a cell, to indicate that the cell contains at least one targeted genetic alteration introduced by man that distinguishes the engineered cell from the naturally occurring cell.

The term “native” is used herein to refer to a cellular component, such as a polynucleotide or polypeptide, that is naturally present in a particular cell. A native polynucleotide or polypeptide is endogenous to the cell.

When used with reference to a polynucleotide or polypeptide, the term “non-native” refers to a polynucleotide or polypeptide that is not naturally present in a particular cell.

When used with reference to the context in which a gene is expressed, the term “non-native” refers to a gene expressed in any context other than the genomic and cellular context in which it is naturally expressed. A gene expressed in a non-native manner may have the same nucleotide sequence as the corresponding gene in a host cell, but may be expressed from a vector or from an integration point in the genome that differs from the locus of the native gene.

The term “heterologous” is used herein to describe a polynucleotide or polypeptide introduced into a host cell. This term encompasses a polynucleotide or polypeptide, respectively, derived from a different organism, species, or strain than that of the host cell. In this case, the heterologous polynucleotide or polypeptide has a sequence that is different from any sequence(s) found in the same host cell. However, the term also encompasses a polynucleotide or polypeptide that has a sequence that is the same as a sequence found in the host cell, wherein the polynucleotide or polypeptide is present in a different context than the native sequence (e.g., a heterologous polynucleotide can be linked to a different promotor and inserted into a different genomic location than that of the native sequence). “Heterologous expression” thus encompasses expression of a sequence that is non-native to the host cell, as well as expression of a sequence that is native to the host cell in a non-native context.

As used with reference to polynucleotides or polypeptides, the term “wild-type” refers to any polynucleotide having a nucleotide sequence, or polypeptide having an amino acid, sequence present in a polynucleotide or polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized. The term “wild-type” is also used to denote naturally occurring cells.

A “control cell” is a cell that is otherwise identical to an engineered cell being tested, including being of the same genus and species as the engineered cell, but lacks the specific genetic modification(s) being tested in the engineered cell. The control cell can include one or more specific modifications that are also present in the engineered cell being tested (i.e., genetic modifications that are not “being tested”).

Enzymes are identified herein by the reactions they catalyze and, unless otherwise indicated, refer to any polypeptide capable of catalyzing the identified reaction. Unless otherwise indicated, enzymes may be derived from any organism and may have a native or mutated amino acid sequence. As is well known, enzymes may have multiple functions and/or multiple names, sometimes depending on the source organism from which they derive. The enzyme names used herein encompass orthologs, including enzymes that may have one or more additional functions or a different name.

The term “feedback-deregulated” is used herein with reference to an enzyme that is normally negatively regulated by a downstream product of the enzymatic pathway (i.e., feedback-inhibition) in a particular cell. In this context, a “feedback-deregulated” enzyme is a form of the enzyme that is less sensitive to feedback-inhibition than the enzyme native to the cell or a form of the enzyme that is native to the cell but is naturally less sensitive to feedback inhibition than one or more other natural forms of the enzyme. A feedback-deregulated enzyme may be produced by introducing one or more mutations into a native enzyme. Alternatively, a feedback-deregulated enzyme may simply be a heterologous, native enzyme that, when introduced into a particular microbial cell, is not as sensitive to feedback-inhibition as the native, native enzyme. In some embodiments, the feedback-deregulated enzyme shows no feedback-inhibition in the microbial cell.

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

The term “titer” as used herein, refers to the mass of a product (e.g., deoxyhydrochorismic acid) present in the culture medium (i.e., extracellular) in a culture of microbial cells divided by the culture volume.

As used herein with respect to recovering deoxyhydrochorismic acid from a cell culture, “recovering” refers to separating the deoxyhydrochorismic acid from at least one other component of the cell culture medium.

As used herein, the phrase “an additional copy of an enzyme” is used herein to refer to an additional copy of a gene encoding the enzyme.

### Engineering Microbes for Deoxyhydrochorismic Acid Production

### Deoxyhydrochorismic Acid Biosynthesis Pathway

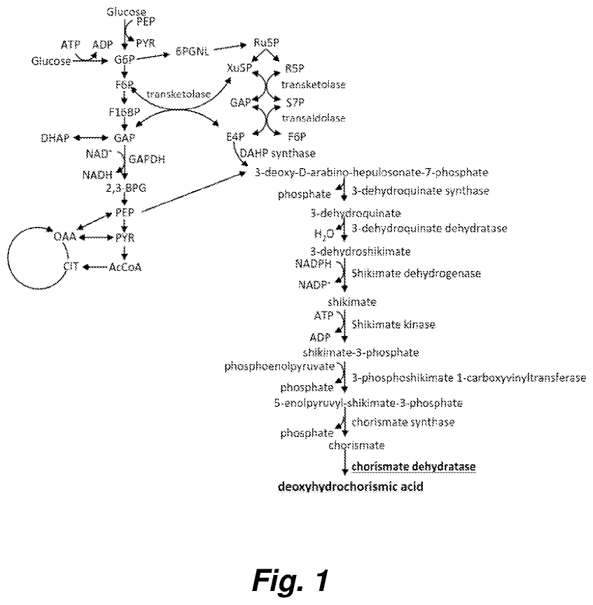

The metabolic pathway to deoxyhydrochorismic acid is derived from the shikimate pathway metabolite, chorismate. (See FIG. 1.) Chorismate is derived from the aromatic branch of amino acid biosynthesis, based on the precursors phosphoenolpyruvate (PEP) and erythrose-4-phosphate (E4P). The first step of the biosynthesis pathway (carried out by 3-deoxy-D-arabinoheptulosonate 7-phosphate [DAHP] synthase) is subject to feedback inhibition by the aromatic amino acids tyrosine, tryptophan, and phenylalanine. The production of deoxyhydrochorismic acid by fermentation of a simple carbon source can be achieved by linking flux through the shikimate biosynthesis pathway to an active chorismate dehydratase, and optionally improving flux through this pathway, in a suitable microbial host.

### Engineering for Microbial Deoxyhydrochorismic Acid Production

Any chorismate dehydratase that is active in the microbial cell being engineered may be introduced into the cell, typically by introducing and expressing the gene(s) encoding the enzyme(s) using standard genetic engineering techniques. Suitable chorismate dehydratases may be derived from any source, including plant, archaeal, fungal, gram-positive bacterial, and gram-negative bacterial sources (see, e.g., those described herein).

One or more copies of any of these genes can be introduced into a selected microbial host cell. If more than one copy of a gene is introduced, the copies can have the same or different nucleotide sequences. In some embodiments, one or both (or all) of the heterologous gene(s) is/are expressed from a strong, constitutive promoter. In some embodiments, the heterologous gene(s) is/are expressed from an inducible promoter. The heterologous gene(s) can optionally be codon-optimized to enhance expression in the selected microbial host cell. The codon-optimization tables used in the Examples are as follows: Bacillus subtilis Kazusa codon table: www.kazusa.or.jp/codon/cgi-bin/showcodon.cgi?species=1423&aa=1&style=N; Yarrowia lipolytica Kazusa codon table: www.kazusa.or.jp/codon/cgi-bin/showcodon.cgi?species=4952&aa=1&style=N; Corynebacterium glutamicum Kazusa codon table: www.kazusa.or.jp/codon/cgi-bin/showcodon.cgi?species=340322&aa=1&style=N; Saccharomyces cerevisiae Kazusa codon table: www.kazusa.or.jp/codon/cgi-bin/showcodon.cgi?species=4932&aa=1&style=N. Also used, was a modified, combined codon usage scheme for S. cereviae and C. glutamicum, which is reproduced below.

Modified Codon Usage Table for Sc and Cg

| Amino | | |
|---|---|---|
| Acid | Codon | Fraction |
| A | GCG | 0.22 |
| A | GCA | 0.29 |
| A | GCT | 0.24 |
| A | GCC | 0.25 |
| C | TGT | 0.36 |
| C | TGC | 0.64 |
| D | GAT | 0.56 |
| D | GAC | 0.44 |
| E | GAG | 0.44 |
| E | GAA | 0.56 |
| F | TTT | 0.37 |
| F | TTC | 0.63 |
| G | GGG | 0.08 |
| G | GGA | 0.19 |
| G | GGT | 0.3 |
| G | GGC | 0.43 |
| H | CAT | 0.32 |
| H | CAC | 0.68 |
| I | ATA | 0.03 |
| I | ATT | 0.38 |
| I | ATC | 0.59 |
| K | AAG | 0.6 |
| K | AAA | 0.4 |
| L | TTG | 0.29 |
| L | TTA | 0.05 |
| L | CTG | 0.29 |
| L | CTA | 0.06 |
| L | CTT | 0.17 |
| L | CTC | 0.14 |
| M | ATG | 1 |
| N | AAT | 0.33 |
| N | AAC | 0.67 |
| P | CCG | 0.22 |
| P | CCA | 0.35 |
| P | CCT | 0.23 |
| P | CCC | 0.2 |
| Q | CAG | 0.61 |
| Q | CAA | 0.39 |
| R | AGG | 0.11 |
| R | AGA | 0.12 |
| R | CGG | 0.09 |
| R | CGA | 0.17 |
| R | CGT | 0.34 |
| R | CGC | 0.18 |
| S | AGT | 0.08 |
| S | AGC | 0.16 |
| S | TCG | 0.12 |
| S | TCA | 0.13 |
| S | TCT | 0.17 |
| S | TCC | 0.34 |
| T | ACG | 0.14 |
| T | ACA | 0.12 |
| T | ACT | 0.2 |
| T | ACC | 0.53 |
| V | GTG | 0.36 |
| V | GTA | 0.1 |
| V | GTT | 0.26 |
| V | GTC | 0.28 |
| W | TGG | 1 |
| Y | TAT | 0.34 |
| Y | TAC | 0.66 |

### Increasing the Activity of Upstream Enzymes

One approach to increasing deoxyhydrochorismic acid production in a microbial cell that is capable of such production is to increase the activity of one or more upstream enzymes in the deoxyhydrochorismic acid biosynthesis pathway. Upstream pathway enzymes include all enzymes involved in the conversions from a feedstock all the way to a metabolite that can be directly converted to deoxyhydrochorismic acid (e.g., chorismate). Illustrative enzymes, for this purpose, include, but are not limited to, those shown in FIG. 1 in the pathway leading to this metabolite. Suitable upstream pathway genes encoding these enzymes may be derived from any available source, including, for example, those disclosed herein.

In some embodiments, the activity of one or more upstream pathway enzymes is increased by modulating the expression or activity of the native enzyme(s). For example, native regulators of the expression or activity of such enzymes can be exploited to increase the activity of suitable enzymes.

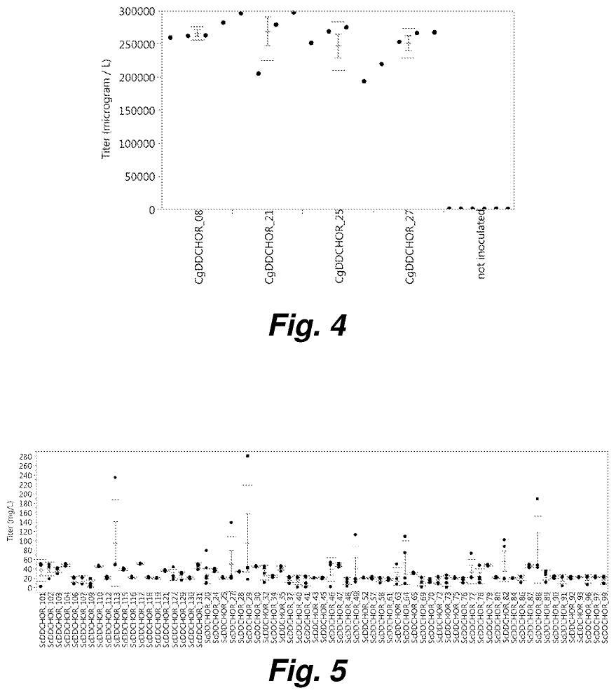

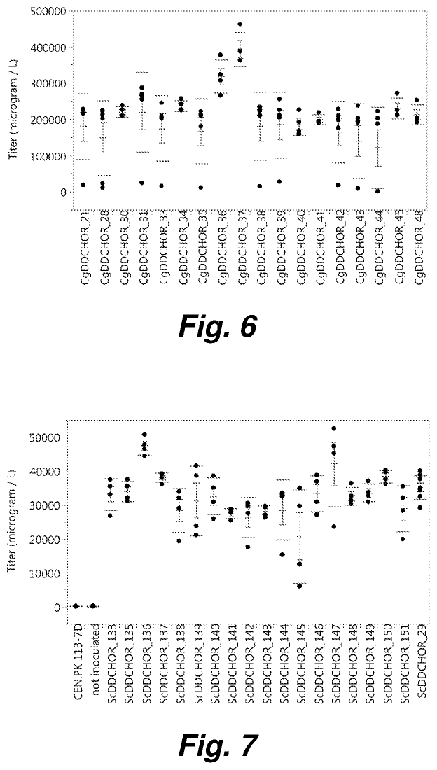

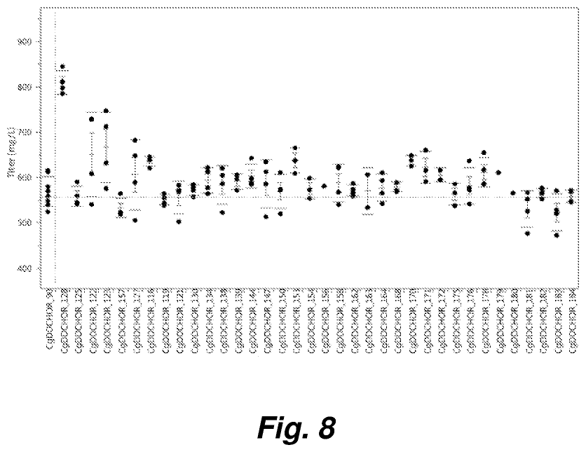

Alternatively, or in addition, one or more promoters can be substituted for native promoters using, for example, a technique such as that illustrated in FIG. 4. In certain embodiments, the replacement promoter is stronger than the native promoter and/or is a constitutive promoter.

In some embodiments, the activity of one or more upstream pathway enzymes is supplemented by introducing one or more of the corresponding genes into the engineered microbial host cell. An introduced upstream pathway gene may be from an organism other than that of the host cell or may simply be an additional copy of a native gene. In some embodiments, one or more such genes are introduced into a microbial host cell capable of deoxyhydrochorismic acid production and expressed from a strong constitutive promoter and/or can optionally be codon-optimized to enhance expression in the selected microbial host cell.

In various embodiments, the engineering of a deoxyhydrochorismic acid-producing microbial cell to increase the activity of one or more upstream pathway enzymes increases the deoxyhydrochorismic acid titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in deoxyhydrochorismic acid titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the deoxyhydrochorismic acid titer observed in a deoxyhydrochorismic acid-producing microbial cell that lacks any increase in activity of upstream pathway enzymes. This reference cell may have one or more other genetic alterations aimed at increasing deoxyhydrochorismic acid production.

In various embodiments, the deoxyhydrochorismic acid titers achieved by increasing the activity of one or more upstream pathway enzymes are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25 gm/L. In various embodiments, the titer is in the range of 50 mg/L to 900 mg/L, 75 mg/L to 850 mg/L, 100 mg/L to 800 mg/L, 200 mg/L to 750 mg/L, 250 mg/L to 700 mg/L, 300 mg/L to 650 mg/L, 350 mg/L to 600 mg/L, or any range bounded by any of the values listed above.

### Introduction of Feedback-Deregulated Enzymes

Since aromatic amino acid biosynthesis is subject to feedback inhibition, another approach to increasing deoxyhydrochorismic acid production in a microbial cell engineered to express a heterologous chorismate dehydratase is to introduce feedback-deregulated forms of one or more enzymes that are normally subject to feedback inhibition in the chorismate dehydratase-expressing microbial cell. DAHP synthase is an example of such an enzyme. A feedback-deregulated form can be a heterologous, wild-type enzyme that is less sensitive to feedback inhibition than the endogenous enzyme in the particular microbial host cell. Alternatively, a feedback-deregulated form can be a variant of an endogenous or heterologous enzyme that has one or more mutations rendering it less sensitive to feedback inhibition than the corresponding wild-type enzyme. Examples of the latter include variant DAHP synthases (two from S. cerevisiae, one from E. coli) that have known point mutations rendering them resistant to feedback inhibition, e.g., S. cerevisiae ARO4Q166K, S. cerevisiae ARO4K229L, and E. coli AroGD146N. The last 5 characters of these designations indicate amino acid substitutions, using the standard one-letter code for amino acids, with the first letter referring to the wild-type residue and the last letter referring to the replacement reside; the numbers indicate the position of the amino acid substitution in the translated protein.

In various embodiments, the engineering of a chorismate dehydratase-expressing microbial cell to express a feedback-deregulated enzymes increases the deoxyhydrochorismic acid titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold. In various embodiments, the increase in deoxyhydrochorismic acid titer is in the range of 10 percent to 100-fold, 2-fold to 50-fold, 5-fold to 40-fold, 10-fold to 30-fold, or any range bounded by any of the values listed above. These increases are determined relative to the deoxyhydrochorismic acid titer observed in a deoxyhydrochorismic acid-producing microbial cell that does not express a feedback-deregulated enzyme. This reference cell may (but need not) have other genetic alterations aimed at increasing deoxyhydrochorismic acid production, i.e., the cell may have increased activity of an upstream pathway enzyme resulting from some means other than feedback-insensitivity.

In various embodiments, the deoxyhydrochorismic acid titers achieved by using a feedback-deregulated enzyme to increase flux though the deoxyhydrochorismic acid biosynthetic pathway are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25 gm/L. In various embodiments, the titer is in the range of 50 mg/L to 900 mg/L, 75 mg/L to 850 mg/L, 100 mg/L to 800 mg/L, 200 mg/L to 750 mg/L, 250 mg/L to 700 mg/L, 300 mg/L to 650 mg/L, 350 mg/L to 600 mg/L, or any range bounded by any of the values listed above.

The approaches of supplementing the activity of one or more endogenous enzymes and/or introducing one or more feedback-deregulated enzymes can be combined in chorismate dehydratase-expressing microbial cells to achieve even higher deoxyhydrochorismic acid production levels.

Reduction of Consumption of Deoxyhydrochorismic Acid and/or Its Precursors

Another approach to increasing deoxyhydrochorismic acid production in a microbial cell that is capable of such production is to decrease the activity of one or more enzymes that consume one or more deoxyhydrochorismic acid pathway precursors or that consume deoxyhydrochorismic acid itself, such as enzymes that produce the amino acids tyrosine, phenylalanine and tryptophan. In an illustrative embodiment, the activity or expression of dihydroxyacetone phosphatase that consumes the deoxyhydrochorismic acid precursor dihydroxyacetone phosphate and converts it to dihydroxyacetone is reduced. In some embodiments, the activity of one or more such enzymes is reduced by modulating the expression or activity of the native enzyme(s). The activity of such enzymes can be decreased, for example, by substituting the native promoter of the corresponding gene(s) with a less active or inactive promoter or by deleting the corresponding gene(s).

Another approach to increasing deoxyhydrochorismic acid production in a microbial cell that is capable of such production is to increase the level of the deoxyhydrochorismic acid precursor phosphoenolpyruvate (PEP) levels by uncoupling the uptake of glucose from the conversion of PEP to pyruvate which occurs by phosphoenolpyruvate phosphotransferase. In some bacteria, phosphoenolpyruvate phosphotransferase activity is provided by the “PTS system,” which consists of three genes, ptsG, ptsH, and ptsI. Deletion or decreased expression of any one of the phosphoenolpyruvate phosphotransferase genes if present eliminates or decreases the activity of the PTS system and improves PEP availability for DAHP synthase.

In various embodiments, the engineering of a deoxyhydrochorismic acid-producing microbial cell to reduce precursor, or deoxyhydrochorismic acid, consumption by one or more side pathways increases the deoxyhydrochorismic acid titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in deoxyhydrochorismic acid titer is in the range of 10-fold to 1000-fold, 20-fold to 500-fold, 50-fold to 400-fold, 10-fold to 300-fold, or any range bounded by any of the values listed above. These increases are determined relative to the deoxyhydrochorismic acid titer observed in a deoxyhydrochorismic acid-producing microbial cell that does not include genetic alterations to reduce precursor consumption. This reference cell may (but need not) have other genetic alterations aimed at increasing deoxyhydrochorismic acid production, i.e., the cell may have increased activity of an upstream pathway enzyme.

In various embodiments, the deoxyhydrochorismic acid titers achieved by reducing precursor, or deoxyhydrochorismic acid, consumption are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25 gm/L. In various embodiments, the titer is in the range of 50 mg/L to 900 mg/L, 75 mg/L to 850 mg/L, 100 mg/L to 800 mg/L, 200 mg/L to 750 mg/L, 250 mg/L to 700 mg/L, 300 mg/L to 650 mg/L, 350 mg/L to 600 mg/L, or any range bounded by any of the values listed above.

### Increasing the NADPH Supply

Another approach to increasing deoxyhydrochorismic acid production in a microbial cell that is capable of such production is to increase the supply of the reduced form of nicotinamide adenine dinucleotide phosphate (NADPH), which provides the reducing equivalents for biosynthetic reactions. For example, the activity of one or more enzymes that increase the NADPH supply can be increased by means similar to those described above for upstream pathway enzymes, e.g., by modulating the expression or activity of the native enzyme(s), replacing the native promoter(s) with a stronger and/or constitutive promoter, and/or introducing one or more gene(s) encoding enzymes that increase the NADPH supply. Illustrative enzymes, for this purpose, include, but are not limited to, pentose phosphate pathway enzymes, NADP+-dependent glyceraldehyde 3-phosphate dehydrogenase (GAPDH), and NADP+-dependent glutamate dehydrogenase.

Such enzymes may be derived from any available source, including any of those described herein with respect to other enzymes. Examples include the NADPH-dependent glyceraldehyde 3-phosphate dehydrogenase (GAPDH) encoded by gapC from Clostridium acetobutylicum, the NADPH-dependent GAPDH encoded by gapB from Bacillus subtilis, and the non-phosphorylating GAPDH encoded by gapN from Streptococcus mutans.

In various embodiments, the engineering of a deoxyhydrochorismic acid-producing microbial cell to increase the activity of one or more of such enzymes increases the deoxyhydrochorismic acid titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in deoxyhydrochorismic acid titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the deoxyhydrochorismic acid titer observed in a deoxyhydrochorismic acid-producing microbial cell that lacks any increase in activity of such enzymes. This reference cell may have one or more other genetic alterations aimed at increasing deoxyhydrochorismic acid production.

In various embodiments, the deoxyhydrochorismic acid titers achieved by reducing precursor, or deoxyhydrochorismic acid, consumption are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25 gm/L. In various embodiments, the titer is in the range of 50 mg/L to 900 mg/L, 75 mg/L to 850 mg/L, 100 mg/L to 800 mg/L, 200 mg/L to 750 mg/L, 250 mg/L to 700 mg/L, 300 mg/L to 650 mg/L, 350 mg/L to 600 mg/L, or any range bounded by any of the values listed above.

Any of the approaches for increasing deoxyhydrochorismic acid production described above can be combined, in any combination, to achieve even higher deoxyhydrochorismic acid production levels.

### Illustrative Amino Acid and Nucleotide Sequences

The following table identifies amino acid and nucleotide sequences used in Example 1. The corresponding sequences are shown in the Sequence Listing.

SEQ ID NO Cross-Reference Table

| | AA | NT |
|---|---|---|
| | SEQ | SEQ |
| Enzyme Description | ID NO: | ID NO: |
| Chorismate dehydratase from Paenibacillus sp. | 1 | 18 |
| oral taxon 786 str. D14 (UniProt ID C6J436) | | |
| Chorismate dehydratase from Paenibacillus sp. | 2 | 19 |
| (strain JDR-2) (UniProt ID C6CUC4) | | |
| Chorismate dehydratase from Pedobacter heparinus | 3 | 20 |
| (UniProt ID C6XW11) | | |
| 3-dehydroquinate synthase from Corynebacterium | 4 | 21 |
| glutamicum ATCC 13032 (UniProt ID Q9X5D2) | | |
| Shikimate kinase from Corynebacterium | 5 | 22 |
| glutamicum ATCC 13032 (UniProt ID Q9X5D1) | | |
| Feedback-deregulated variant of a DAHP synthase | 6 | 23 |
| from Saccharomyces cerevisiae (UniProt ID | | |
| P32449) including K229L | | |
| Chorismate dehydratase from Streptomyces griseus | 7 | 24 |
| (UniProt ID B1W536) | | |
| Chorismate dehydratase from Streptomyces coelicolor | 8 | 25 |
| (UniProt ID Q9L0T8) | | |
| Chorismate dehydratase from Streptomyces sp Mg1 | 9 | 26 |
| (UniProt ID B4V2Z2) | | |
| Chorismate dehydratase from Streptomyces collinus | 10 | 27 |
| (UniProt ID S5V7C6) | | |
| Chorismate dehydratase from Salinispora arenicola | 11 | 28 |
| (UniProt ID A8M634) | | |
| Chorismate dehydratase from Streptomyces | 12 | 29 |
| leeuwenhoekii UniProt ID A0A0F7VYE2) | | |
| Chorismate dehydratase Leptospira mayottensis | 13 | 30 |
| (UniProt ID M6VLB7) | | |
| Feedback-deregulated variant of a DAHP synthase | 14 | 31 |
| from Escherichia coli K12 (UniProt | | |
| ID P00888) including N8K | | |
| Feedback-deregulated variant of a DAHP synthase | 15 | 32 |
| from Escherichia coli K12 (UniProt | | |
| ID P0AB91) including P150L | | |
| Chorismate dehydratase from Streptomyces caniferus | 16 | 33 |
| (Uniprot ID A0A128ATQ8) | | |
| Chorismate dehydratase from Desulfovibrio vulgaris | 17 | 34 |
| subsp. vulgaris (strain DP4) (Uniprot ID | | |
| A0A0H3A518) | | |

### Microbial Host Cells

Any microbe that can be used to express introduced genes can be engineered for fermentative production of deoxyhydrochorismic acid as described above. In certain embodiments, the microbe is one that is naturally incapable of fermentative production of deoxyhydrochorismic acid. In some embodiments, the microbe is one that is readily cultured, such as, for example, a microbe known to be useful as a host cell in fermentative production of compounds of interest. Bacteria cells, including gram-positive or gram-negative bacteria can be engineered as described above. Examples include, in addition to C. glutamicum cells, Bacillus subtilus, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., P. alcaligenes, P. citrea, Lactobacilis spp. (such as L. lactis, L. plantarum), L. grayi, E. coli, E. faecium, E. gallinarum, E. casseliflavus, and/or E. faecalis cells.

There are numerous types of anaerobic cells that can be used as microbial host cells in the methods described herein. In some embodiments, the microbial cells are obligate anaerobic cells. Obligate anaerobes typically do not grow well, if at all, in conditions where oxygen is present. It is to be understood that a small amount of oxygen may be present, that is, there is some level of tolerance level that obligate anaerobes have for a low level of oxygen. Obligate anaerobes engineered as described above can be grown under substantially oxygen-free conditions, wherein the amount of oxygen present is not harmful to the growth, maintenance, and/or fermentation of the anaerobes.

Alternatively, the microbial host cells used in the methods described herein can be facultative anaerobic cells. Facultative anaerobes can generate cellular ATP by aerobic respiration (e.g., utilization of the TCA cycle) if oxygen is present. However, facultative anaerobes can also grow in the absence of oxygen. Facultative anaerobes engineered as described above can be grown under substantially oxygen-free conditions, wherein the amount of oxygen present is not harmful to the growth, maintenance, and/or fermentation of the anaerobes, or can be alternatively grown in the presence of greater amounts of oxygen.

In some embodiments, the microbial host cells used in the methods described herein are filamentous fungal cells. (See, e.g., Berka & Barnett, Biotechnology Advances, (1989), 7(2):127-154). Examples include Trichoderma longibrachiatum, T. viride, T. koningii, T. harzianum, Penicillium sp., Humicola insolens, H. lanuginose, H. grisea, Chrysosporium sp., C. lucknowense, Gliocladium sp., Aspergillus sp. (such as A. oryzae, A. niger, A. sojae, A. japonicus, A. nidulans, or A. awamori), Fusarium sp. (such as F. roseum, F. graminum F. cerealis, F. oxysporuim, or F. venenatum), Neurospora sp. (such as N. crassa or Hypocrea sp.), Mucor sp. (such as M. miehei), Rhizopus sp., and Emericella sp. cells. In particular embodiments, the fungal cell engineered as described above is A. nidulans, A. awamori, A. oryzae, A. aculeatus, A. niger, A. japonicus, T. reesei, T. viride, F. oxysporum, or F. solani. Illustrative plasmids or plasmid components for use with such hosts include those described in U.S. Patent Pub. No. 2011/0045563.

Yeasts can also be used as the microbial host cell in the methods described herein. Examples include: Saccharomyces sp., Schizosaccharomyces sp., Pichia sp., Hansenula polymorpha, Pichia stipites, Kluyveromyces marxianus, Kluyveromyces spp., Yarrowia lipolytica and Candida sp. In some embodiments, the Saccharomyces sp. is S. cerevisiae (See, e.g., Romanos et al., Yeast, (1992), 8(6):423-488). Illustrative plasmids or plasmid components for use with such hosts include those described in U.S. Pat. No. 7,659,097 and U.S. Patent Pub. No. 2011/0045563.

In some embodiments, the host cell can be an algal cell derived, e.g., from a green alga, red alga, a glaucophyte, a chlorarachniophyte, a euglenid, a chromista, or a dinoflagellate. (See, e.g., Saunders & Warmbrodt, “Gene Expression in Algae and Fungi, Including Yeast,” (1993), National Agricultural Library, Beltsville, Md.). Illustrative plasmids or plasmid components for use in algal cells include those described in U.S. Patent Pub. No. 2011/0045563.

In other embodiments, the host cell is a cyanobacterium, such as cyanobacterium classified into any of the following groups based on morphology: Chlorococcales, Pleurocapsales, Oscillatoriales, Nostocales, Synechosystic or Stigonematales (See, e.g., Lindberg et al., Metab. Eng., (2010) 12(1):70-79). Illustrative plasmids or plasmid components for use in cyanobacterial cells include those described in U.S. Patent Pub. Nos. 2010/0297749 and 2009/0282545 and in Intl. Pat. Pub. No. WO 2011/034863.

### Genetic Engineering Methods

Microbial cells can be engineered for fermentative deoxyhydrochorismic acid production using conventional techniques of molecular biology (including recombinant techniques), microbiology, cell biology, and biochemistry, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994); Singleton et al., Dictionary of Microbiology and Molecular Biology 2nd ed., J. Wiley & Sons (New York, N.Y. 1994).

Vectors are polynucleotide vehicles used to introduce genetic material into a cell. Vectors useful in the methods described herein can be linear or circular. Vectors can integrate into a target genome of a host cell or replicate independently in a host cell. For many applications, integrating vectors that produced stable transformants are preferred. Vectors can include, for example, an origin of replication, a multiple cloning site (MCS), and/or a selectable marker. An expression vector typically includes an expression cassette containing regulatory elements that facilitate expression of a polynucleotide sequence (often a coding sequence) in a particular host cell. Vectors include, but are not limited to, integrating vectors, prokaryotic plasmids, episomes, viral vectors, cosmids, and artificial chromosomes.

Illustrative regulatory elements that may be used in expression cassettes include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif. (1990).

In some embodiments, vectors may be used to introduce systems that can carry out genome editing, such as CRISPR systems. See U.S. Patent Pub. No. 2014/0068797, published 6 Mar. 2014; see also Jinek M., et al., “A programmable dual-RNA-guided DNA endonuclease in adaptive bacterial immunity,” Science 337:816-21, 2012). In Type II CRISPR-Cas9 systems, Cas9 is a site-directed endonuclease, namely an enzyme that is, or can be, directed to cleave a polynucleotide at a particular target sequence using two distinct endonuclease domains (HNH and RuvC/RNase H-like domains). Cas9 can be engineered to cleave DNA at any desired site because Cas9 is directed to its cleavage site by RNA. Cas9 is therefore also described as an “RNA-guided nuclease.” More specifically, Cas9 becomes associated with one or more RNA molecules, which guide Cas9 to a specific polynucleotide target based on hybridization of at least a portion of the RNA molecule(s) to a specific sequence in the target polynucleotide. Ran, F. A., et al., (“In vivo genome editing using Staphylococcus aureus Cas9,” Nature 520(7546): 186-91, 2015, April 9], including all extended data) present the crRNA/tracrRNA sequences and secondary structures of eight Type II CRISPR-Cas9 systems. Cas9-like synthetic proteins are also known in the art (see U.S. Published Patent Application No. 2014-0315985, published 23 Oct. 2014).

Example 1 describes illustrative integration approaches for introducing polynucleotides and other genetic alterations into the genomes of S. cerevisiae and C. glutamicum cells.

Vectors or other polynucleotides can be introduced into microbial cells by any of a variety of standard methods, such as transformation, conjugation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Patent Pub. Nos. 2009/0203102, 2010/0048964, and 2010/0003716, and International Publication Nos. WO 2009/076676, WO 2010/003007, and WO 2009/132220.

### Engineered Microbial Cells

The above-described methods can be used to produce engineered microbial cells that produce, and in certain embodiments, overproduce, deoxyhydrochorismic acid. Engineered microbial cells can have at least 1, 2, 3, 4, 5, 6, 7, 8, 9, 10, 20, 30, 40, 50, 60, 70, 80, 90, 100 or more genetic alterations, such as 30-100 alterations, as compared to a native microbial cell, such as any of the microbial host cells described herein. Engineered microbial cells described in the Example below have one, two, or three genetic alterations, but those of skill in the art can, following the guidance set forth herein, design microbial cells with additional alterations. In some embodiments, the engineered microbial cells have not more than 15, 14, 13, 12, 11, 10, 9, 8, 7, 6, 5, or 4 genetic alterations, as compared to a native microbial cell. In various embodiments, microbial cells engineered for deoxyhydrochorismic acid production can have a number of genetic alterations falling within the any of the following illustrative ranges: 1-10, 1-9, 1-8, 2-7, 2-6, 2-5, 2-4, 2-3, 3-7, 3-6, 3-5, 3-4, etc.

In some embodiments, an engineered microbial cell expresses at least one heterologous (e.g., non-native) gene, e.g., a chorismate dehydratase gene. In various embodiments, the microbial cell can include and express, for example: (1) a single chorismate dehydratase gene, (2) two or more heterologous chorismate dehydratase genes, which can be the same or different (in other words, multiple copies of the same heterologous chorismate dehydratase gene can be introduced or multiple, different heterologous chorismate dehydratase genes can be introduced), (3) a single heterologous chorismate dehydratase gene that is not native to the cell and one or more additional copies of a native chorismate dehydratase gene (if applicable), or (4) two or more non-native chorismate dehydratase genes, which can be the same or different, and/or one or more additional copies of a native chorismate dehydratase gene (if applicable).

In certain embodiments, this engineered host cell can include at least one additional genetic alteration that increases flux through any pathway leading to the production of an immediate precursor of deoxyhydrochorismic acid. As discussed above, this can be accomplished by one or more of the following: increasing the activity of upstream enzymes, e.g., by introducing a feedback-deregulated version of a DAHP synthase, alone or in combination with other means for increasing the activity of upstream enzymes.

The engineered microbial cells can contain introduced genes that have a native nucleotide sequence or that differ from native. For example, the native nucleotide sequence can be codon-optimized for expression in a particular host cell. Codon optimization for a particular host can, for example, be based on the codon usage tables found at www.kazusa.or.jp/codon/. The amino acid sequences encoded by any of these introduced genes can be native or can differ from native. In various embodiments, the amino acid sequences have at least 60 percent, 70 percent, 75 percent, 80 percent, 85 percent, 90 percent, 95 percent or 100 percent amino acid sequence identity with a native amino acid sequence.

The approach described herein has been carried out in yeast cells, namely S. cerevisiae, and in bacterial cells, namely C. glutamicum (See Example 1.)

### Illustrative Engineered Yeast Cells

In certain embodiments, the engineered yeast (e.g., S. cerevisiae) cell expresses one or more non-native chorismate dehydratase(s) having at least 70 percent, 75 percent, 80 percent, 85 percent, 90 percent, 95 percent or 100 percent amino acid sequence identity with a chorismate dehydratase from Paenibacillus sp. oral taxon 786 str. D14 (UniProt ID C6J436); and/or one or more non-native chorismate dehydratase(s) having at least 70 percent, 75 percent, 80 percent, 85 percent, 90 percent, 95 percent or 100 percent amino acid sequence identity with a chorismate dehydratase from Paenibacillus sp. (strain JDR-2) (UniProt ID C6CUC4); and/or one or more non-native chorismate dehydratase(s) having at least 70 percent, 75 percent, 80 percent, 85 percent, 90 percent, 95 percent or 100 percent amino acid sequence identity with a chorismate dehydratase from Pedobacter heparinus (UniProt ID C6XW11); and/or one or more non-native 3-dehydroquinate synthase(s) having at least 70 percent, 75 percent, 80 percent, 85 percent, 90 percent, 95 percent or 100 percent amino acid sequence identity with a 3-dehydroquinate synthase from Corynebacterium glutamicum ATCC 13032 (UniProt ID Q9X5D2); and/or one or more non-native shikimate kinase(s) having at least 70 percent, 75 percent, 80 percent, 85 percent, 90 percent, 95 percent or 100 percent amino acid sequence identity with a shikimate kinase from C. glutamicum ATCC 13032 (UniProt ID Q9X5D2); and/or one or more feedback-deregulated variant(s) having at least 70 percent, 75 percent, 80 percent, 85 percent, 90 percent, 95 percent or 100 percent amino acid sequence identity with a feedback deregulated variant of an S. cerevisiae DAHP synthase (UniProt ID P32449) including the amino acid substitution K229L.

In particular embodiments:
the chorismate dehydratase from Paenibacillus sp. oral taxon 786 str. D14 (UniProt ID C6J436) includes SEQ ID NO:1;
the chorismate dehydratase from Paenibacillus sp. (strain JDR-2) (UniProt ID C6CUC4) includes SEQ ID NO:2;
the chorismate dehydratase from Pedobacter heparinus (UniProt ID C6XW11) includes SEQ ID NO:3;
the 3-dehydroquinate synthase(s) from C. glutamicum ATCC 13032 (UniProt ID Q9X5D2) includes SEQ ID NO:4;
the shikimate kinase from C. glutamicum ATCC 13032 (UniProt ID Q9X5D2) includes SEQ ID NO:5;
the feedback-deregulated DAHP synthase from S. cerevisiae (UniProt ID P32449), harboring amino acid substitution K229L, includes SEQ ID NO:6.

In an illustrative embodiment, a titer of about 525 mg/L was achieved after engineering S. cerevisiae to express chorismate dehydratase from Paenibacillus sp. oral taxon 786 str. D14 (UniProt ID C6J436) (SEQ ID NO:1), chorismate dehydratase from Pedobacter heparinus (UniProt ID C6XW11) (SEQ ID NO:3); 3-dehydroquinate synthase from C. glutamicum ATCC 13032 (UniProt ID Q9X5D2) (SEQ ID NO:4), shikimate kinase from C. glutamicum ATCC 13032 (UniProt ID Q9X5D2) (SEQ ID NO:5), feedback-deregulated DAHP synthase from S. cerevisiae (UniProt ID P32449), harboring amino acid substitution K229L, (SEQ ID NO:6).

### Illustrative Engineered Bacterial Cells

In certain embodiments, the engineered bacterial (e.g., C. glutamicum) cell expresses one or more (e.g., two) non-native chorismate dehydratase(s) having at least 70 percent, 75 percent, 80 percent, 85 percent, 90 percent, 95 percent or 100 percent amino acid sequence identity with a chorismate dehydratase from Streptomyces griseus (UniProt ID B1W536); and/or one or more non-native chorismate dehydratase(s) having at least 70 percent, 75 percent, 80 percent, 85 percent, 90 percent, 95 percent or 100 percent amino acid sequence identity with a chorismate dehydratase from Streptomyces coelicolor (UniProt ID Q9LOT8); and/or one or more non-native chorismate dehydratase(s) having at least 70 percent, 75 percent, 80 percent, 85 percent, 90 percent, 95 percent or 100 percent amino acid sequence identity with a chorismate dehydratase from Streptomyces sp Mg1 (UniProt ID B4V2Z2); and/or one or more non-native chorismate dehydratase(s) having at least 70 percent, 75 percent, 80 percent, 85 percent, 90 percent, 95 percent or 100 percent amino acid sequence identity with a chorismate dehydratase from Streptomyces collinus (UniProt ID S5V7C6); and/or one or more non-native chorismate dehydratase(s) having at least 70 percent, 75 percent, 80 percent, 85 percent, 90 percent, 95 percent or 100 percent amino acid sequence identity with a chorismate dehydratase from Salinispora arenicola (UniProt ID A8M634); and/or one or more non-native chorismate dehydratase(s) having at least 70 percent, 75 percent, 80 percent, 85 percent, 90 percent, 95 percent or 100 percent amino acid sequence identity with a chorismate dehydratase from Streptomyces leeuwenhoekii (UniProt ID AOAOF7VYE2); and/or one or more non-native chorismate dehydratase(s) having at least 70 percent, 75 percent, 80 percent, 85 percent, 90 percent, 95 percent or 100 percent amino acid sequence identity with a chorismate dehydratase from Leptospira mayottensis (UniProt ID M6VLB7); and/or one or more non-native chorismate dehydratase(s) having at least 70 percent, 75 percent, 80 percent, 85 percent, 90 percent, 95 percent or 100 percent amino acid sequence identity with a chorismate dehydratase from Paenibacillus sp. (strain JDR-2) (UniProt ID C6CUC4); and/orone or more feedback-deregulated variant(s) having at least 70 percent, 75 percent, 80 percent, 85 percent, 90 percent, 95 percent or 100 percent amino acid sequence identity with a feedback-deregulated variant of an Escherichia coli K12 DAHP synthase (UniProt ID P00888) including amino acid substitution N8K and/or with a feedback-deregulated variant of an Escherichia coli K12 DAHP synthase ((UniProt ID POAB91) including P150L.

In particular embodiments:
the chorismate dehydratase from Streptomyces griseus (UniProt ID B1W536) includes SEQ ID NO:7;
the chorismate dehydratase from chorismate dehydratase from Streptomyces coelicolor (UniProt ID Q9LOT8) includes SEQ ID NO:8;
the chorismate dehydratase from Streptomyces sp Mg1 (UniProt ID B4V2Z2) includes SEQ ID NO:9;
the chorismate dehydratase from Streptomyces collinus (UniProt ID S5V7C6) includes SEQ ID NO:10;
the chorismate dehydratase from Salinispora arenicola (UniProt ID A8M634) includes SEQ ID NO:11;
the chorismate dehydratase from Streptomyces leeuwenhoekii (UniProt ID A0AOF7VYE2) includes SEQ ID NO:12;
the chorismate dehydratase from Leptospira mayottensis (UniProt ID M6VLB7) includes SEQ ID NO:13;
the chorismate dehydratase from Paenibacillus sp. (strain JDR-2) (UniProt ID C6CUC4) includes SEQ ID NO:2;
the feedback-deregulated DAHP synthase from Escherichia coli K12 (UniProt ID P00888), harboring amino acid substitution N8K, includes SEQ ID NO:14; and/or the feedback-deregulated DAHP synthase from Escherichia coli K12 (UniProt ID POAB91), harboring amino acid substitution P150L, includes SEQ ID NO:15.

In an illustrative embodiment, a titer of about 450 mg/L was achieved after engineering C. glutamicum to express two copies of a gene encoding chorismate dehydratase from Streptomyces griseus (UniProt ID B1W536) (SEQ ID NO:7) and feedback-deregulated DAHP synthase from Escherichia coli K12 (UniProt ID POAB91), harboring amino acid substitution P150L (SEQ ID NO:15).

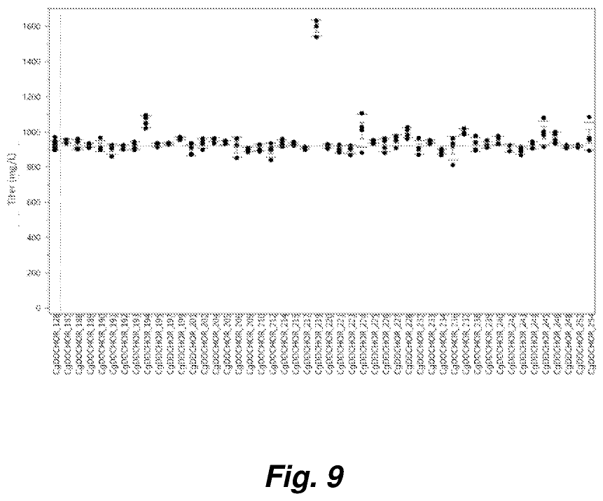

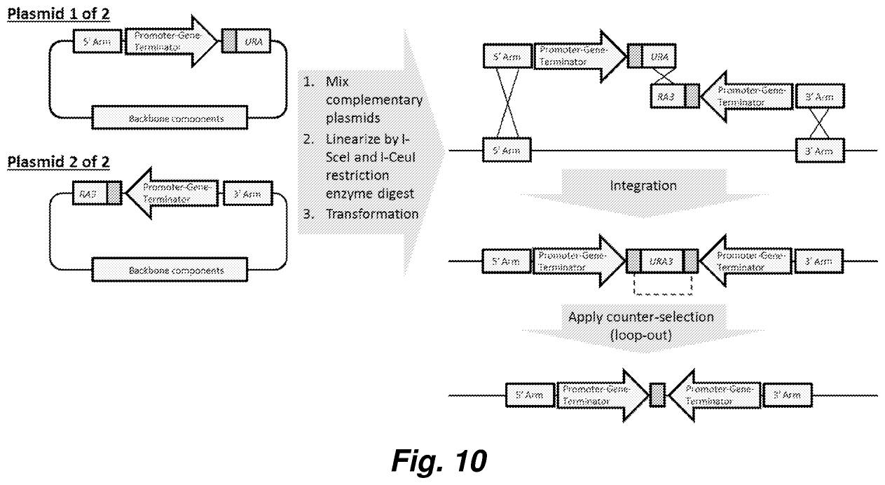

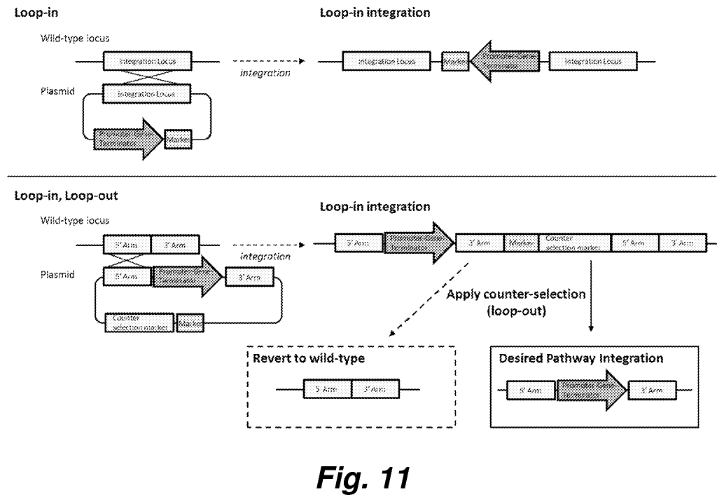

This strain, CgDDCHOR_37, was further engineered to yield a titer of about 1600 mg/L (see FIG. 9, strain CgDDCHOR_128.) Accordingly, in further improved, illustrative embodiments, the engineered bacterial (e.g., C. glutamicum) cell additionally expresses one or more (e.g., two) non-native chorismate dehydratase(s) having at least 70 percent, 75 percent, 80 percent, 85 percent, 90 percent, 95 percent or 100 percent amino acid sequence identity with a chorismate dehydratase from Strepomyces caniferus (Uniprot ID A0A128ATQ8), and/or from Desulfovibrio vulgaris subsp. vulgaris (strain DP4) (Uniprot ID A0A0H3A518), and/or from Paenibacillus sp. (strain JDR-2) (UniProt ID C6CUC4). In some embodiments, a further improved, illustrative strain expresses at least one copy of each of these three enzymes or two copies of each of these three enzymes.

In particular embodiments:
the chorismate dehydratase from Strepomyces caniferus (Uniprot ID A0A128ATQ8) includes SEQ ID NO:16;
the chorismate dehydratase from Desulfovibrio vulgaris subsp. vulgaris (strain DP4) (Uniprot ID A0A0H3A518) includes SEQ ID NO:17; and/or the chorismate dehydratase from Paenibacillus sp. (strain JDR-2) (UniProt ID C6CUC4) includes SEQ ID NO:2.

### Culturing of Engineered Microbial Cells

Any of the microbial cells described herein can be cultured, e.g., for maintenance, growth, and/or deoxyhydrochorismic acid production.

In some embodiments, the cultures are grown to an optical density at 600 nm of 10-500, such as an optical density of 50-150.

In various embodiments, the deoxyhydrochorismic acid titers achieved by reducing precursor, or deoxyhydrochorismic acid, consumption are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25 gm/L. In various embodiments, the titer is in the range of 50 mg/L to 900 mg/L, 75 mg/L to 850 mg/L, 100 mg/L to 800 mg/L, 200 mg/L to 750 mg/L, 250 mg/L to 700 mg/L, 300 mg/L to 650 mg/L, 350 mg/L to 600 mg/L, or any range bounded by any of the values listed above.

### Culture Media

Microbial cells can be cultured in any suitable medium including, but not limited to, a minimal medium, i.e., one containing the minimum nutrients possible for cell growth. Minimal medium typically contains: (1) a carbon source for microbial growth; (2) salts, which may depend on the particular microbial cell and growing conditions; and (3) water. Suitable media can also include any combination of the following: a nitrogen source for growth and product formation, a sulfur source for growth, a phosphate source for growth, metal salts for growth, vitamins for growth, and other cofactors for growth.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (levulose), and galactose; illustrative oligosaccharides include dextran or glucan, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids.

Minimal medium can be supplemented with one or more selective agents, such as antibiotics.

To produce deoxyhydrochorismic acid, the culture medium can include, and/or is supplemented during culture with, glucose and/or a nitrogen source such as urea, an ammonium salt, ammonia, or any combination thereof.

### Culture Conditions

Materials and methods suitable for the maintenance and growth of microbial cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass.

In general, cells are grown and maintained at an appropriate temperature, gas mixture, and pH (such as about 20° C. to about 37° C., about 6% to about 84% CO2, and a pH between about 5 to about 9). In some aspects, cells are grown at 35° C. In certain embodiments, such as where thermophilic bacteria are used as the host cells, higher temperatures (e.g., 50° C.- 75° C.) may be used. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fed-batch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer

### Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that can be consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at the rate that can be supported by the amount of sugar in the cell medium. In some embodiments, sugar does not accumulate during the time the cells are cultured. In various embodiments, the cells are cultured under limited sugar conditions for times greater than or about 1, 2, 3, 5, 10, 15, 20, 25, 30, 35, 40, 50, 60, or 70 hours or even up to about 5-10 days. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some aspects, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. In some cultures, significantly higher levels of sugar (e.g., glucose) are used, e.g., at least 10% (w/v), 20% (w/v), 30% (w/v), 40% (w/v), 50% (w/v), 60% (w/v), 70% (w/v), or up to the solubility limit for the sugar in the medium. In some embodiments, the sugar levels falls within a range of any two of the above values, e.g.: 0.1-10% (w/v), 1.0-20% (w/v), 10-70% (w/v), 20-60% (w/v), or 30-50% (w/v). Furthermore, different sugar levels can be used for different phases of culturing. For fed-batch culture (e.g., of S. cerevisiae or C. glutamicum), the sugar level can be about 100-200 g/L (10-20% (w/v)) in the batch phase and then up to about 500-700 g/L (50-70% in the feed).

Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), or 0.02% (w/v) yeast extract. In some cultures, significantly higher levels of yeast extract can be used, e.g., at least 1.5% (w/v), 2.0% (w/v), 2.5% (w/v), or 3% (w/v). In some cultures (e.g., of S. cerevisiae or C. glutamicum), the yeast extract level falls within a range of any two of the above values, e.g.: 0.5-3.0% (w/v), 1.0-2.5% (w/v), or 1.5-2.0% (w/v).

### Deoxyhydrochorismic Acid Production and Recovery

Any of the methods described herein may further include a step of recovering deoxyhydrochorismic acid. In some embodiments, the produced deoxyhydrochorismic acid contained in a so-called harvest stream is recovered/harvested from the production vessel. The harvest stream may include, for instance, cell-free or cell-containing aqueous solution coming from the production vessel, which contains deoxyhydrochorismic acid as a result of the conversion of production substrate by the resting cells in the production vessel. Cells still present in the harvest stream may be separated from the deoxyhydrochorismic acid by any operations known in the art, such as for instance filtration, centrifugation, decantation, membrane crossflow ultrafiltration or microfiltration, tangential flow ultrafiltration or microfiltration or dead-end filtration. After this cell separation operation, the harvest stream is essentially free of cells.

Further steps of separation and/or purification of the produced deoxyhydrochorismic acid from other components contained in the harvest stream, i.e., so-called downstream processing steps may optionally be carried out. These steps may include any means known to a skilled person, such as, for instance, concentration, extraction, crystallization, precipitation, adsorption, ion exchange, and/or chromatography. Any of these procedures can be used alone or in combination to purify deoxyhydrochorismic acid.

Further purification steps can include one or more of, e.g., concentration, crystallization, precipitation, washing and drying, treatment with activated carbon, ion exchange, nanofiltration, and/or re-crystallization. The design of a suitable purification protocol may depend on the cells, the culture medium, the size of the culture, the production vessel, etc. and is within the level of skill in the art.

The following examples are given for the purpose of illustrating various embodiments of the disclosure and are not meant to limit the present disclosure in any fashion. Changes therein and other uses which are encompassed within the spirit of the disclosure, as defined by the scope of the claims, will be identifiable to those skilled in the art.

### EXAMPLE 1—Construction and Selection of Strains of Saccharomyces cerevisiae and Corynebacterium glutamicum Engineered to Produce deoxyhydrochorismic acid

### Plasmid/DNA Design

All strains tested for this work were transformed with plasmid DNA designed using proprietary software. Plasmid designs were specific to one of the two host organisms engineered in this work. The plasmid DNA was physically constructed by a standard DNA assembly method. This plasmid DNA was then used to integrate metabolic pathway inserts by one of two host-specific methods, each described below.

S. cerevisiae Pathway Integration

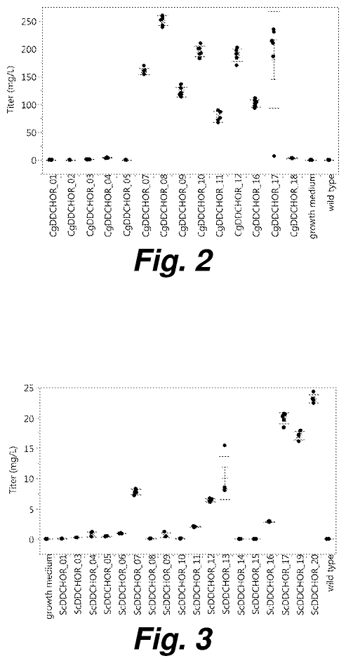

A “split-marker, double-crossover” genomic integration strategy has been developed to engineer S. cerevisiae strains. FIG. 2 illustrates genomic integration of complementary, split-marker plasmids and verification of correct genomic integration via colony PCR in S. cerevisiae. Two plasmids with complementary 5′ and 3′ homology arms and overlapping halves of a URA3 selectable marker (direct repeats shown by the hashed bars) were digested with meganucleases and transformed as linear fragments. A triple-crossover event integrated the desired heterologous genes into the targeted locus and re-constituted the full URA3 gene. Colonies derived from this integration event were assayed using two 3-primer reactions to confirm both the 5′ and 3′ junctions (UF/IF/wt-R and DR/IF/wt-F). For strains in which further engineering is desired, the strains can be plated on 5-FOA plates to select for the removal of URA3, leaving behind a small single copy of the original direct repeat. This genomic integration strategy can be used for gene knock-out, gene knock-in, and promoter titration in the same workflow.

C. glutamicum Pathway Integration

A “loop-in, single-crossover” genomic integration strategy has been developed to engineer C. glutamicum strains. FIG. 3 illustrates genomic integration of loop-in only and loop-in/loop-out constructs and verification of correct integration via colony PCR. Loop-in only constructs (shown under the heading “Loop-in”) contained a single 2-kb homology arm (denoted as “integration locus”), a positive selection marker (denoted as “Marker”)), and gene(s) of interest (denoted as “promoter-gene-terminator”). A single crossover event integrated the plasmid into the C. glutamicum chromosome. Integration events are stably maintained in the genome by growth in the presence of antibiotic (25 μg/ml kanamycin). Correct genomic integration in colonies derived from loop-in integration were confirmed by colony PCR with UF/IR and DR/IF PCR primers.

Loop-in, loop-out constructs (shown under the heading “Loop-in, loop-out) contained two 2-kb homology arms (5′ and 3′ arms), gene(s) of interest (arrows), a positive selection marker (denoted “Marker”), and a counter-selection marker. Similar to “loop-in” only constructs, a single crossover event integrated the plasmid into the chromosome of C. glutamicum. Note: only one of two possible integrations is shown here. Correct genomic integration was confirmed by colony PCR and counter-selection was applied so that the plasmid backbone and counter-selection marker could be excised. This results in one of two possibilities: reversion to wild-type (lower left box) or the desired pathway integration (lower right box). Again, correct genomic loop-out is confirmed by colony PCR. (Abbreviations: Primers: UF=upstream forward, DR=downstream reverse, IR=internal reverse, IF=internal forward.)

### Cell Culture

Separate workflows were established for C. glutamicum and S. cerevisiae due to differences in media requirements and growth. Both processes involved a hit-picking step that consolidated successfully built strains using an automated workflow that randomized strains across the plate. For each strain that was successfully built, up to four replicates were tested from distinct colonies to test colony-to-colony variation and other process variation. If fewer than four colonies were obtained, the existing colonies were replicated so that at least four wells were tested from each desired genotype.

The colonies were consolidated into 96-well plates with selective medium (BHI for C. glutamicum, SD-ura for S. cerevisiae) and cultivated for two days until saturation and then frozen with 16.6% glycerol at −80° ° C. for storage. The frozen glycerol stocks were then used to inoculate a seed stage in minimal media with a low level of amino acids to help with growth and recovery from freezing. The seed plates were grown at 30° C. for 1-2 days. The seed plates were then used to inoculate a main cultivation plate with minimal medium and grown for 48-88 hours. Plates were removed at the desired time points and tested for cell density (OD600), viability and glucose, supernatant samples stored for LC-MS analysis for product of interest.

### Cell Density

Cell density was measured using a spectrophotometric assay detecting absorbance of each well at 600 nm. Robotics were used to transfer fixed amounts of culture from each cultivation plate into an assay plate, followed by mixing with 175 mM sodium phosphate (pH 7.0) to generate a 10-fold dilution. The assay plates were measured using a Tecan M1000 spectrophotometer and assay data uploaded to a LIMS database. A non-inoculated control was used to subtract background absorbance. Cell growth was monitored by inoculating multiple plates at each stage, and then sacrificing an entire plate at each time point.

To minimize settling of cells while handling large number of plates (which could result in a non-representative sample during measurement) each plate was shaken for 10-15 seconds before each read. Wide variations in cell density within a plate may also lead to absorbance measurements outside of the linear range of detection, resulting in underestimate of higher OD cultures. In general, the tested strains so far have not varied significantly enough for this be a concern.

### Cell Viability

Two methods were used to measure cell viability. The first assay utilized a single stain, propidium iodide, to assess cell viability. Propidium iodide binds to DNA and is permeable to cells with compromised cell membranes. Cells that take up the propidium iodide are considered non-viable. A dead cell control was used to normalize to total number of cells, by incubating a cell sample of control culture at 95° C. for 10 minutes. These control samples and test samples were incubated with the propidium iodide stain for 5 minutes, washed twice with 175 mM phosphate buffer, and fluorescence measured in black solid-bottom 96-well plates at 617 nm.

### Glucose

Glucose is measured using an enzymatic assay with 16U/mL glucose oxidase (Sigma) with 0.2 U/mL horseradish peroxidase (Sigma) and 0.2 mM Amplex red in 175 mM sodium phosphate buffer, pH 7. Oxidation of glucose generates hydrogen peroxide, which is then oxidized to reduce Amplex red, which changes absorbance at 560 nm. The change is absorbance is correlated to the glucose concentration in the sample using standards of known concentration.

### Liquid-Solid Separation

To harvest extracellular samples for analysis by LC-MS, liquid and solid phases were separated via centrifugation. Cultivation plates were centrifuged at 2000 rpm for 4 minutes, and the supernatant was transferred to destination plates using robotics. 75u L of supernatant was transferred to each plate, with one stored at 4ºC, and the second stored at 80° C. for long-term storage.

### Genetic Engineering Approach and Results

A library approach was taken to identify functional enzymes in both Saccharomyces cerevisiae and Corynebacterium glutamicum. A broad search of chorismate dehydratase sequences identified in total 18 orthologous sequences from these sources: 5 archaeal and 13 bacterial. These chorismate dehydratase enzymes were codon-optimized and expressed in both hosts.

### First Round of Engineering

Deoxyhydrochorismic acid titers were achieved in both host strains in the initial POC experiments. In C. glutamicum, a 250 mg/L titer was produced in the first round of engineering by integration of the chorismate dehydratase from Streptomyces griseus (UniProt ID B1W536). (Table 1, FIG. 2.) In S. cerevisiae, a 24 mg/L titer was produced in the first round of engineering by integration of the chorismate dehydratase gene from Paenibacillus sp. oral taxon 786 str. D14 (UniProt ID C6J436). (Table 1, FIG. 3.)

The chorismate dehydratases from Streptomyces coelicolor (UniProt ID Q9LOT8), Streptomyces sp Mg1 (UniProt ID B4V2Z2), Streptomyces collinus (UniProt ID S5V7C6), Salinispora arenicola (UniProt ID A8M634), Streptomyces leeuwenhoekii (UniProt ID AOAOF7VYE2), Leptospira mayottensis (UniProt ID M6VLB7) and Paenibacillus sp. (UniProt ID C6CUC4) are also active in C. glutamicum and enable production of 100-200 mg/L deoxyhydrochorismic acid.

The chorismate dehydratases from Paenibacillus sp. (strain JDR-2) (UniProt ID C6CUC4), and Pedobacter heparinus (UniProt ID C6XW11) are also active in S. cerevisiae and enable the production of 15-20 mg/L deoxyhydrochorismic acid.

TABLE 1
First-Round Results

| | | | | | |
|---|---|---|---|---|---|
| | | E1 | Enzyme 1 - | Enzyme 1 - | E1 Codon |
| | Titer | Uniprot | activity | source | Optimization |
| Strain name | (μg/L) | ID | name | organism | Abbrev. |

Corynebacterium glutamicum

| | | | | | |
|---|---|---|---|---|---|
| CgDDCHOR_01 | 549.6 | A1RU54 | chorismate | Pyrobaculum islandicum | combined Sc and Cg |
| | | | dehydratase | (strain DSM 4184/JCM | codon usage |
| | | | | 9189/GEO3) | |
| CgDDCHOR_02 | 392.3 | D2RH69 | chorismate | Archaeoglobus profundus | combined Sc and Cg |
| | | | dehydratase | (strain DSM 5631/JCM | codon usage |
| | | | | 9629/NBRC 100127/ | |
| | | | | Av18) | |
| CgDDCHOR_03 | 1512.3 | A8M924 | chorismate | Caldivirga maquilingensis | combined Sc and Cg |
| | | | dehydratase | (strain ATCC 700844/ | codon usage |
| | | | | DSM 13496/JCM 10307/ | |
| | | | | IC-167) | |
| CgDDCHOR_04 | 5740.0 | A0A075H1C1 | chorismate | uncultured marine group | combined Sc and Cg |
| | | | dehydratase | II/III euryarchaeote | codon usage |
| | | | | KM3_28_D12 | |
| CgDDCHOR_05 | 278.0 | A0A124IV87 | chorismate | Vulcanisaeta sp. CIS_19 | combined Sc and Cg |
| | | | dehydratase | | codon usage |
| CgDDCHOR_07 | 123408.1 | Q9L0T8 | chorismate | Streptomyces coelicolor | combined Sc and Cg |
| | | | dehydratase | (strain ATCC BAA-471/ | codon usage |
| | | | | A3(2)/M145) | |
| CgDDCHOR_08 | 257014.3 | B1W536 | chorismate | Streptomyces griseus | combined Sc and Cg |
| | | | dehydratase | subsp. griseus | codon usage |
| | | | | (strain JCM 4626/ | |
| | | | | NBRC 13350) | |
| CgDDCHOR_09 | 95299.9 | B4V2Z2 | chorismate | Streptomyces sp. Mg1 | combined Sc and Cg |
| | | | dehydratase | | codon usage |
| CgDDCHOR_10 | 148620.1 | S5V7C6 | chorismate | Streptomyces collinus | combined Sc and Cg |
| | | | dehydratase | (strain DSM 40733/Tu | codon usage |
| | | | | 365) | |
| CgDDCHOR_11 | 61452.9 | A8M634 | chorismate | Salinispora arenicola | combined Sc and Cg |
| | | | dehydratase | (strain CNS-205) | codon usage |
| CgDDCHOR_12 | 147889.0 | A0A0F7VYE2 | chorismate | Streptomyces | combined Sc and Cg |
| | | | dehydratase | leeuwenhoekii | codon usage |
| CgDDCHOR_16 | 79828.5 | M6VLB7 | chorismate | Leptospira mayottensis | combined Sc and Cg |
| | | | dehydratase | 200901116 | codon usage |
| CgDDCHOR_17 | 138116.7 | C6CUC4 | chorismate | Paenibacillus sp. | combined Sc and Cg |
| | | | dehydratase | (strain JDR-2) | codon usage |
| CgDDCHOR_18 | 4366.2 | Q5SK49 | chorismate | Thermus thermophilus | combined Sc and Cg |
| | | | dehydratase | (strain HB8/ATCC 27634/ | codon usage |
| | | | | DSM 579) | |

Saccharomyces cerevisiae

| | | | | | |
|---|---|---|---|---|---|
| ScDDCHOR_01 | 72.6 | A1RU54 | chorismate | Pyrobaculum islandicum | combined Sc and Cg |
| | | | dehydratase | (strain DSM 4184/JCM | codon usage |
| | | | | 9189/GEO3) | |
| ScDDCHOR_03 | 248.6 | A8M924 | chorismate | Caldivirga maquilingensis | combined Sc and Cg |
| | | | dehydratase | (strain ATCC 700844/ | codon usage |
| | | | | DSM 13496/JCM 10307/ | |
| | | | | IC-167) | |
| ScDDCHOR_04 | 775.9 | A0A075H1C1 | chorismate | uncultured marine group | combined Sc and Cg |
| | | | dehydratase | II/III euryarchaeote | codon usage |
| | | | | KM3_28_D12 | |
| ScDDCHOR_05 | 462.6 | A0A124IV87 | chorismate | Vulcanisaeta sp. CIS_19 | combined Sc and Cg |
| | | | dehydratase | | codon usage |
| ScDDCHOR_06 | 943.2 | A0A075HZV4 | chorismate | uncultured marine group | combined Sc and Cg |
| | | | dehydratase | II/III euryarchaeote | codon usage |
| | | | | KM3_98_B01 | |
| ScDDCHOR_07 | 7809.3 | Q9LOT8 | chorismate | Streptomyces coelicolor | combined Sc and Cg |
| | | | dehydratase | (strain ATCC BAA-471/ | codon usage |
| | | | | A3(2)/M145) | |
| ScDDCHOR_08 | 74.3 | B1W536 | chorismate | Streptomyces griseus | combined Sc and Cg |
| | | | dehydratase | subsp. griseus | codon usage |
| | | | | (strain JCM 4626/ | |
| | | | | NBRC 13350) | |
| ScDDCHOR_09 | 614.3 | B4V2Z2 | chorismate | Streptomyces sp. Mg1 | combined Sc and Cg |
| | | | dehydratase | | codon usage |
| ScDDCHOR_10 | 91.3 | S5V7C6 | chorismate | Streptomyces collinus | combined Sc and Cg |
| | | | dehydratase | (strain DSM 40733/Tu | codon usage |
| | | | | 365) | |
| ScDDCHOR_11 | 2039.4 | A8M634 | chorismate | Salinispora arenicola | combined Sc and Cg |
| | | | dehydratase | (strain CNS-205) | codon usage |
| ScDDCHOR_12 | 6438.6 | A0A0F7VYE2 | chorismate | Streptomyces | combined Sc and Cg |
| | | | dehydratase | leeuwenhoekii | codon usage |
| ScDDCHOR_13 | 10853.1 | F2RII7 | chorismate | Streptomyces venezuelae | combined Sc and Cg |
| | | | dehydratase | (strain ATCC 10712/CBS | codon usage |
| | | | | 650.69/DSM 40230/JCM | |
| | | | | 4526/NBRC 13096/PD | |
| | | | | 04745) | |
| ScDDCHOR_14 | 1.6 | O25468 | chorismate | Helicobacter pylori | combined Sc and Cg |
| | | | dehydratase | (strain ATCC 700392/26695) | codon usage |
| | | | | (Campylobacter pylori) | |
| ScDDCHOR_15 | 5.4 | A1W0R9 | chorismate | Campylobacter jejuni | combined Sc and Cg |
| | | | dehydratase | subsp. jejuni serotype | codon usage |
| | | | | O:23/36 (strain 81-176) | |
| ScDDCHOR_16 | 2859.3 | M6VLB7 | chorismate | Leptospira mayottensis | combined Sc and Cg |
| | | | dehydratase | 200901116 | codon usage |
| ScDDCHOR_17 | 19935.7 | C6CUC4 | chorismate | Paenibacillus sp. | combined Sc and Cg |
| | | | dehydratase | (strain JDR-2) | codon usage |
| ScDDCHOR_19 | 17346.4 | C6XW11 | chorismate | Pedobacter heparinus | combined Sc and Cg |
| | | | dehydratase | (strain ATCC 13125/DSM | codon usage |
| | | | | 2366/NCIB 9290) | |
| ScDDCHOR_20 | 24250.0 | C6J436 | chorismate | Paenibacillus sp. | combined Sc and Cg |
| | | | dehydratase | oral taxon 786 str. D14 | codon usage |

We introduced additional genetic changes to the best performing strains of each C. glutamicum and S. cerevisiae to improve production of deoxyhydrochorismic acid. We took a combinatorial library approach to introduce an additional copy of 1-3 upstream pathway genes and chorismate dehydratase, in separate daughter strains, under the control of a strong, constitutive promoters (Tables 2-3 show the results of second and third rounds of genetic engineering). Upstream pathway genes represent all genes involved in the conversion of key precursors (i.e. E4P & PEP) into the last native metabolite (e.g., chorismate) in the pathway leading to deoxyhydrochorismate. Enzymes successfully built into strains and tested in the combinatorial library approach are shown in the deoxyhydrochorismic acid pathway diagram (FIG. 1).

### Second Round of Engineering

In C. glutamicum, the most improved strain from the second round of genetic engineering contained an additional copy of chorismate dehydratase from Streptomyces griseus (UniProt ID B1W536). (Table 2, FIG. 4.)

In S. cerevisiae the most improved strain from the second round of genetic engineering contained (in addition to chorismate dehydratase gene from Paenibacillus sp. oral taxon 786 str. D14 (UniProt ID C6J436)) shikimate kinase from C. glutamicum ATCC 13032 (UniProt ID Q9X5D1), 3-dehydroquinate synthase (UniProt ID Q9X5D2) from C. glutamicum ATCC 13032, and DAHP synthase (UniProt ID P32449) from S. cerevisiae containing the amino acid substitution K229L which reduces pathway feedback inhibition. (Table 2, FIG. 5)

### Third Round of Engineering

In the third round of genetic engineering, the best C. glutamicum strain from the second round of engineering was further improved. In C. glutamicum, the most improved strain from the third round of genetic engineering also included a feedback deregulated DAHP synthase (UniProt ID P00888) from E. coli K12 containing the amino acid substitution P150L, and the second-most improved strain contained the feedback deregulated DAHP synthase (UniProt ID POAB91) from E. coli K12 containing the amino acid substitution N8K.

In addition to expressing additional upstream pathway enzymes, to further improve deoxyhydrochorismic acid production in C. glutamicum, increasing flux from glucose to E4P, the precursor to the shikimate pathway by deletion of the PTS glucose uptake system (PTS-) is also expected to improve production of deoxyhydrochorismic acid [1, 2].

In the third round of genetic engineering, the best S. cerevisiae strain from the second round of engineering was further improved. In S. cerevisiae, the most improved strain from the third round of genetic engineering contained chorismate dehydratase from Pedobacter heparinus ATCC 13125 (UniProt ID C6XW11), and the second-most improved strain contained chorismate dehydratase (UniProt ID C6CUC4) from Paenibacillus sp. strain JDR-2.

In addition to expressing additional upstream pathway enzymes, to further improve deoxyhydrochorismic acid production in S. cerevisiae and C. glutamicum it is anticipated that 1) replacing the native promoters of enzymes that consume deoxyhydrochorismic acid pathway metabolites (e.g., enzymes to make amino acids tyrosine, phenylalanine and tryptophan) to lower the activity of these enzymes and 2) improving NADPH cofactor availability will be beneficial.

Fourth Round of Engineering of Corynebacterium glutamicum

In a fourth round of genetic engineering of C. glutamicum, the best C. glutamicum strain from the third round of engineering (CgDDCHOR_37) was further improved. This starting strain included two copies of a chorismate dehydratase from Streptomyces griseus (UniProt ID B1W536) and a feedback-deregulated variant of an Escherichia coli K12 DAHP synthase (UniProt ID POAB91) including P150L.

The best-performing strain from the fourth round of genetic engineering (CgDDCHOR_90) included, in addition to the above alterations, three further chorismate dehydratases: one from Streptomyces caniferus (UniProt ID A0A128ATQ8), one from Disulfovibrio vulgaris (Uniprot ID AOAOH3A518), and one from Paenibacillus sp. (strain JDR-2) (UniProt ID C6CUC4). This strain produced deoxyhydrochorismic acid at a level of about 606 mg/L of culture medium.

Fifth Round of Engineering of Corynebacterium glutamicum

In a fifth round of genetic engineering of C. glutamicum, the best C. glutamicum strain from the fourth round of engineering (CgDDCHOR_90) was further improved.

The best-performing strain from the fifth round of genetic engineering (CgDDCHOR_128) included additional copies of each of the three further chorismate dehydratases found in the fourth round of engineering, i.e., one more from Streptomyces caniferus (UniProt ID A0A128ATQ8), one more from Disulfovibrio vulgaris (Uniprot ID A0A0H3A518), and one more from Paenibacillus sp. (strain JDR-2) (UniProt ID C6CUC4). This strain produced deoxyhydrochorismic acid at a level of about 810 mg/L of culture medium.

TABLE 2
Second-Round Results
In addition the enzymes in this table, the Corynebacterium glutamicum
strains contained chorismate dehydratase (UniProt ID B1W536) and
Saccharomyces cerevisiae strains contained chorismate dehydratase
(UniProt C6J436), which are the best enzymes in each best-performing
host from the first round of genetic engineering (see Table 1).
All of the DAHP synthases (UniProt ID P32449) tested in the second
round contained K229L, which reduces pathway feedback-inhibition.

| | | E1 | | Enzyme 1 - | E1 Codon | E2 | |
|---|---|---|---|---|---|---|---|
| Strain | Titer | Uniprot | Enzyme 1 - | source | Optimization | Uniprot | Enzyme 2 - |
| Name | (mg/L) | ID | activity name | organism | Abbrev. | ID | activity name |
| CgDD | 10.98 | B1W536 | chorismate | Streptomyces | modified | | |
| CHOR_20 | | | dehydratase | griseus subsp. | Corynebacterium | | |
| | | | | griseus JCM | glutamicum | | |
| | | | | 4626 | codon usage | | |
| CgDD | 269.13 | B1W536 | chorismate | Streptomyces | modified | | |
| CHOR_21 | | | dehydratase | griseus subsp. | Corynebacterium | | |
| | | | | griseus JCM | glutamicum | | |
| | | | | 4626 | codon usage | | |
| CgDD | 246.89 | B1W536 | chorismate | Streptomyces | modified | | |
| CHOR_25 | | | dehydratase | griseus subsp. | Corynebacterium | | |
| | | | | griseus JCM | glutamicum | | |
| | | | | 4626 | codon usage | | |
| CgDD | 251.29 | B1W536 | chorismate | Streptomyces | modified | | |
| CHOR_27 | | | dehydratase | griseus subsp. | Corynebacterium | | |
| | | | | griseus JCM | glutamicum | | |
| | | | | 4626 | codon usage | | |
| ScDD | 37.49 | P32449 | DAHP synthase | Saccharomyces | Corynebacterium | Q8NQ64 | Transaldolase |
| CHOR_24 | | | | cerevisiae | glutamicum | | |
| ScDD | 12.46 | Q8NRC0 | Shikimate 5- | Corynebacterium | modified | Q9Z470 | 3-phosphoshikimate |
| CHOR_25 | | | dehydrogenase | glutamicum | codon usage | | 1-carboxyvinyl- |
| | | | | ATCC 13032 | for Cg and Sc | | transferase |
| ScDD | 50.39 | Q9X5D2 | 3-dehydroquinate | Corynebacterium | modified | Q9X5D0 | Chorismate |
| CHOR_27 | | | synthase | glutamicum | codon usage | | synthase (CS) |
| | | | | ATCC 13032 | for Cg and Sc | | |
| ScDD | 34.54 | Q9X5D1 | Shikimate kinase | Corynebacterium | modified | P32449 | DAHP synthase |
| CHOR_28 | | | (SK) | glutamicum | codon usage | | |
| | | | | ATCC 13032 | for Cg and Sc | | |
| ScDD | 95.35 | Q9X5D1 | Shikimate kinase | Corynebacterium | modified | Q9X5D2 | 3-dehydroquinate |
| CHOR_29 | | | (SK) | glutamicum | codon usage | | synthase |
| | | | | ATCC 13032 | for Cg and Sc | | |
| ScDD | 44.55 | Q9X5D2 | 3-dehydroquinate | Corynebacterium | modified | P32449 | DAHP synthase |
| CHOR_30 | | | synthase | glutamicum | codon usage | | |
| | | | | ATCC 13032 | for Cg and Sc | | |
| ScDD | 32.17 | Q9X5D2 | 3-dehydroquinate | Corynebacterium | modified | Q8NRS1 | Enolase |
| CHOR_32 | | | synthase | glutamicum | codon usage | | |
| | | | | ATCC 13032 | for Cg and Sc | | |
| ScDD | 7.79 | P53228 | Transaldolase | Saccharomyces | modified | P53228 | Transaldolase |
| CHOR_34 | | | | cerevisiae | codon usage | | |
| | | | | S288c | for Cg and Sc | | |
| ScDD | 41.96 | Q8NRS1 | Enolase | Corynebacterium | modified | P32449 | DAHP synthase |
| CHOR_35 | | | | glutamicum | codon usage | | |
| | | | | ATCC 13032 | for Cg and Sc | | |
| ScDD | 17.62 | Q9X5D1 | Shikimate kinase | Corynebacterium | modified | Q9Z470 | 3-phosphoshikimate |
| CHOR_37 | | | (SK) | glutamicum | codon usage | | 1-carboxyvinyl- |
| | | | | ATCC 13032 | for Cg and Sc | | transferase |
| ScDD | 16.26 | Q9X5D2 | 3-dehydroquinate | Corynebacterium | modified | Q9Z470 | 3-phosphoshikimate |
| CHOR_40 | | | synthase | glutamicum | codon usage | | 1-carboxyvinyl- |
| | | | | ATCC 13032 | for Cg and Sc | | transferase |
| ScDD | 14.17 | Q9X5D0 | Chorismate | Corynebacterium | modified | Q8NRS1 | Enolase |
| CHOR_41 | | | synthase (CS) | glutamicum | codon usage | | |
| | | | | ATCC 13032 | for Cg and Sc | | |
| ScDD | 20.35 | Q9X5D0 | Chorismate | Corynebacterium | modified | Q8NQ64 | Transaldolase |
| CHOR_43 | | | synthase (CS) | glutamicum | codon usage | | |
| | | | | ATCC 13032 | for Cg and Sc | | |
| ScDD | 20.41 | P08566 | 3-dehydroquinate | Saccharomyces | modified | P53228 | Transaldolase |
| CHOR_45 | | | synthase,3- | cerevisiae | codon usage | | |
| | | | phosphoshikimate | S288c | for Cg and Sc | | |
| | | | 1-carboxyvinyl- | | | | |
| | | | transferase,3- | | | | |
| | | | phosphoshikimate | | | | |
| | | | 1-carboxyvinyl- | | | | |
| | | | transferase, | | | | |
| | | | Shikimate kinase | | | | |
| | | | (SK), Shikimate 5- | | | | |
| | | | dehydrogenase,3- | | | | |
| | | | dehydroquinate | | | | |
| | | | dehydratase (3- | | | | |
| | | | dehydroquinase) | | | | |
| ScDD | 38.67 | Q9X5D2 | 3-dehydroquinate | Corynebacterium | modified | P32449 | DAHP synthase |
| CHOR_46 | | | synthase | glutamicum | codon usage | | |
| | | | | ATCC 13032 | for Cg and Sc | | |
| ScDD | 48.37 | P08566 | 3-dehydroquinate | Saccharomyces | modified | P00924 | Enolase |
| CHOR_47 | | | synthase,3- | cerevisiae | codon usage | | |
| | | | phosphoshikimate | S288c | for Cg and Sc | | |
| | | | 1-carboxyvinyl- | | | | |
| | | | transferase,3- | | | | |
| | | | phosphoshikimate | | | | |
| | | | 1-carboxyvinyl- | | | | |
| | | | transferase, | | | | |
| | | | Shikimate kinase | | | | |
| | | | (SK), Shikimate 5- | | | | |
| | | | dehydrogenase,3- | | | | |
| | | | dehydroquinate | | | | |
| | | | dehydratase (3- | | | | |
| | | | dehydroquinase) | | | | |
| ScDD | 13.72 | P00924 | Enolase | Saccharomyces | modified | | |
| CHOR_48 | | | | cerevisiae | codon usage | | |
| | | | | S288c | for Cg and Sc | | |
| ScDD | 40.01 | P53228 | Transaldolase | Saccharomyces | modified | P28777 | Chorismate |
| CHOR_49 | | | | cerevisiae | codon usage | | synthase (CS) |
| | | | | S288c | for Cg and Sc | | |
| ScDD | 20.78 | Q9X5D0 | Chorismate | Corynebacterium | modified | Q8NQ64 | Transaldolase |
| CHOR_52 | | | synthase (CS) | glutamicum | codon usage | | |
| | | | | ATCC 13032 | for Cg and Sc | | |
| ScDD | 20.65 | Q9X5D2 | 3-dehydroquinate | Corynebacterium | modified | Q9X5D0 | Chorismate |
| CHOR_57 | | | synthase | glutamicum | codon usage | | synthase (CS) |
| | | | | ATCC 13032 | for Cg and Sc | | |
| ScDD | 17.55 | Q9X5D1 | Shikimate kinase | Corynebacterium | modified | Q9X5D0 | Chorismate |
| CHOR_58 | | | (SK) | glutamicum | codon usage | | synthase (CS) |
| | | | | ATCC 13032 | for Cg and Sc | | |
| ScDD | 17.31 | Q9X5D2 | 3-dehydroquinate | Corynebacterium | modified | Q9X5D0 | Chorismate |
| CHOR_61 | | | synthase | glutamicum | codon usage | | synthase (CS) |
| | | | | ATCC 13032 | for Cg and Sc | | |
| ScDD | 23.42 | Q9X5D1 | Shikimate kinase | Corynebacterium | modified | Q9X5D2 | 3-dehydroquinate |
| CHOR_63 | | | (SK) | glutamicum | codon usage | | synthase |
| | | | | ATCC 13032 | for Cg and Sc | | |
| ScDD | 52.69 | Q9X5D2 | 3-dehydroquinate | Corynebacterium | modified | Q8NRS1 | Enolase |
| CHOR_64 | | | synthase | glutamicum | codon usage | | |
| | | | | ATCC 13032 | for Cg and Sc | | |
| ScDD | 23.12 | Q9X5D1 | Shikimate kinase | Corynebacterium | modified | P32449 | DAHP synthase |
| CHOR_65 | | | (SK) | glutamicum | codon usage | | |
| | | | | ATCC 13032 | for Cg and Sc | | |
| ScDD | 13.76 | Q8NQ64 | Transaldolase | Corynebacterium | modified | Q8NRC0 | Shikimate 5- |
| CHOR_69 | | | | glutamicum | codon usage | | dehydrogenase |
| | | | | ATCC 13032 | for Cg and Sc | | |
| ScDD | 9.73 | Q8NQ64 | Transaldolase | Corynebacterium | modified | | |
| CHOR_70 | | | | glutamicum | codon usage | | |
| | | | | ATCC 13032 | for Cg and Sc | | |
| ScDD | 15.68 | Q9X5D2 | 3-dehydroquinate | Corynebacterium | modified | Q8NQ64 | Transaldolase |
| CHOR_72 | | | synthase | glutamicum | codon usage | | |
| | | | | ATCC 13032 | for Cg and Sc | | |
| ScDD | 14.11 | Q9X5D1 | Shikimate kinase | Corynebacterium | modified | Q8NQ64 | Transaldolase |
| CHOR_73 | | | (SK) | glutamicum | codon usage | | |
| | | | | ATCC 13032 | for Cg and Sc | | |
| ScDD | 20.14 | Q9X5D2 | 3-dehydroquinate | Corynebacterium | modified | Q8NQ64 | Transaldolase |
| CHOR_75 | | | synthase | glutamicum | codon usage | | |
| | | | | ATCC 13032 | for Cg and Sc | | |
| ScDD | 16.45 | Q8NQ64 | Transaldolase | Corynebacterium | modified | Q8NRC0 | Shikimate 5- |
| CHOR_76 | | | | glutamicum | codon usage | | dehydrogenase |
| | | | | ATCC 13032 | for Cg and Sc | | |
| ScDD | 34.24 | Q9X5D1 | Shikimate kinase | Corynebacterium | modified | Q9X5D0 | Chorismate |
| CHOR_77 | | | (SK) | glutamicum | codon usage | | synthase (CS) |
| | | | | ATCC 13032 | for Cg and Sc | | |
| ScDD | 24.28 | Q9X5D0 | Chorismate | Corynebacterium | modified | Q8NRC0 | Shikimate 5- |
| CHOR_78 | | | synthase (CS) | glutamicum | codon usage | | dehydrogenase |
| | | | | ATCC 13032 | for Cg and Sc | | |
| ScDD | 28.78 | Q9X5D2 | 3-dehydroquinate | Corynebacterium | modified | P32449 | DAHP synthase |
| CHOR_79 | | | synthase | glutamicum | codon usage | | |
| | | | | ATCC 13032 | for Cg and Sc | | |
| ScDD | 21.46 | Q9X5D1 | Shikimate kinase | Corynebacterium | modified | Q8NRC0 | Shikimate 5- |
| CHOR_80 | | | (SK) | glutamicum | codon usage | | dehydrogenase |
| | | | | ATCC 13032 | for Cg and Sc | | |
| ScDD | 56.84 | Q8NRS1 | Enolase | Corynebacterium | modified | Q8NQ64 | Transaldolase |
| CHOR_82 | | | | glutamicum | codon usage | | |
| | | | | ATCC 13032 | for Cg and Sc | | |
| ScDD | 19.82 | Q9X5D2 | 3-dehydroquinate | Corynebacterium | modified | Q8NQ64 | Transaldolase |
| CHOR_84 | | | synthase | glutamicum | codon usage | | |
| | | | | ATCC 13032 | for Cg and Sc | | |
| ScDD | 20.20 | P08566 | 3-dehydroquinate | Saccharomyces | modified | P28777 | Chorismate |
| CHOR_86 | | | synthase,3- | cerevisiae | codon usage | | synthase (CS) |
| | | | phosphoshikimate | S288c | for Cg and Sc | | |
| | | | 1-carboxyvinyl- | | | | |
| | | | transferase,3- | | | | |
| | | | phosphoshikimate | | | | |
| | | | 1-carboxyvinyl- | | | | |
| | | | transferase, | | | | |
| | | | Shikimate kinase | | | | |
| | | | (SK), Shikimate 5- | | | | |
| | | | dehydrogenase,3- | | | | |
| | | | dehydroquinate | | | | |
| | | | dehydratase (3- | | | | |
| | | | dehydroquinase) | | | | |
| ScDD | 45.16 | P53228 | Transaldolase | Saccharomyces | modified | P32449 | DAHP synthase |
| CHOR_87 | | | | cerevisiae | codon usage | | |
| | | | | S288c | for Cg and Sc | | |
| ScDD | 81.54 | P00924 | Enolase | Saccharomyces | modified | P32449 | DAHP synthase |
| CHOR_88 | | | | cerevisiae | codon usage | | |
| | | | | S288c | for Cg and Sc | | |
| ScDD | 26.68 | P14843 | Phospho-2- | Saccharomyces | modified | P14843Z | Phospho-2- |
| CHOR_89 | | | dehydro-3- | cerevisiae | codon usage | | dehydro-3- |
| | | | deoxyheptonate | S288c | for Cg and Sc | | deoxyheptonate |
| | | | aldolase | | | | aldolase |
| ScDD | 22.31 | Q9Z470 | 3-phosphoshikimate | Corynebacterium | modified | Q9Z470 | 3-phosphoshikimate |
| CHOR_90 | | | 1-carboxyvinyl- | glutamicum | codon usage | | 1-carboxyvinyl- |
| | | | transferase | ATCC 13032 | for Cg and Sc | | transferase |
| ScDD | 17.04 | Q9X5D2 | 3-dehydroquinate | Corynebacterium | modified | Q9X5D2 | 3-dehydroquinate |
| CHOR_91 | | | synthase | glutamicum | codon usage | | synthase |
| | | | | ATCC 13032 | for Cg and Sc | | |
| ScDD | 21.49 | Q9X5D1 | Shikimate kinase | Corynebacterium | modified | Q9X5D1 | Shikimate kinase |
| CHOR_92 | | | (SK) | glutamicum | codon usage | | (SK) |
| | | | | ATCC 13032 | for Cg and Sc | | |
| ScDD | 22.38 | Q9X5D0 | Chorismate | Corynebacterium | modified | Q9X5D0 | Chorismate |
| CHOR_93 | | | synthase (CS) | glutamicum | codon usage | | synthase (CS) |
| | | | | ATCC 13032 | for Cg and Sc | | |
| ScDD | 18.85 | Q8NRC0 | Shikimate 5- | Corynebacterium | modified | Q8NRC0 | Shikimate 5- |
| CHOR_96 | | | dehydrogenase | glutamicum | codon usage | | dehydrogenase |
| | | | | ATCC 13032 | for Cg and Sc | | |
| ScDD | 22.60 | Q8NRS1 | Enolase | Corynebacterium | modified | Q8NRS1 | Enolase |
| CHOR_97 | | | | glutamicum | codon usage | | |
| | | | | ATCC 13032 | for Cg and Sc | | |
| ScDD | 15.63 | P08566 | 3-dehydroquinate | Saccharomyces | modified | P00924 | Enolase |
| CHOR_99 | | | synthase,3- | cerevisiae | codon usage | | |
| | | | phosphoshikimate | S288c | for Cg and Sc | | |
| | | | 1-carboxyvinyl- | | | | |
| | | | transferase,3- | | | | |
| | | | phosphoshikimate | | | | |
| | | | 1-carboxyvinyl- | | | | |
| | | | transferase, | | | | |
| | | | Shikimate kinase | | | | |
| | | | (SK),Shikimate 5- | | | | |
| | | | dehydrogenase,3- | | | | |
| | | | dehydroquinate | | | | |
| | | | dehydratase (3- | | | | |
| | | | dehydroquinase) | | | | |
| ScDD | 37.21 | P08566 | 3-dehydroquinate | Saccharomyces | modified | P32449 | DAHP synthase |
| CHOR_101 | | | synthase,3- | cerevisiae | codon usage | | |
| | | | phosphoshikimate | S288c | for Cg and Sc | | |
| | | | 1-carboxyvinyl- | | | | |
| | | | transferase,3- | | | | |
| | | | phosphoshikimate | | | | |
| | | | 1-carboxyvinyl- | | | | |
| | | | transferase, | | | | |
| | | | Shikimate kinase | | | | |
| | | | (SK), Shikimate 5- | | | | |
| | | | dehydrogenase,3- | | | | |
| | | | dehydroquinate | | | | |
| | | | dehydratase (3- | | | | |
| | | | dehydroquinase) | | | | |
| ScDD | 39.89 | Q9X5D2 | 3-dehydroquinate | Corynebacterium | modified | P32449 | DAHP synthase |
| CHOR_102 | | | synthase | glutamicum | codon usage | | |
| | | | | ATCC 13032 | for Cg and Sc | | |
| ScDD | 37.99 | P53228 | Transaldolase | Saccharomyces | modified | P00924 | Enolase |
| CHOR_103 | | | | cerevisiae | codon usage | | |
| | | | | S288c | for Cg and Sc | | |
| ScDD | 47.90 | Q8NRS1 | Enolase | Corynebacterium | modified | P32449 | DAHP synthase |
| CHOR_104 | | | | glutamicum | codon usage | | |
| | | | | ATCC 13032 | for Cg and Sc | | |
| ScDD | 17.40 | Q9X5D2 | 3-dehydroquinate | Corynebacterium | modified | Q8NRS1 | Enolase |
| CHOR_106 | | | synthase | glutamicum | codon usage | | |
| | | | | ATCC 13032 | for Cg and Sc | | |
| ScDD | 17.88 | Q9X5D0 | Chorismate | Corynebacterium | modified | Q8NRS1 | Enolase |
| CHOR_107 | | | synthase (CS) | glutamicum | codon usage | | |
| | | | | ATCC 13032 | for Cg and Sc | | |
| ScDD | 8.43 | Q9X5D1 | Shikimate kinase | Corynebacterium | modified | Q8NRS1 | Enolase |
| CHOR_109 | | | (SK) | glutamicum | codon usage | | |
| | | | | ATCC 13032 | for Cg and Sc | | |
| ScDD | 45.48 | Q8NRS1 | Enolase | Corynebacterium | modified | P32449 | DAHP synthase |
| CHOR_110 | | | | glutamicum | codon usage | | |
| | | | | ATCC 13032 | for Cg and Sc | | |
| ScDD | 21.70 | Q9X5D2 | 3-dehydroquinate | Corynebacterium | modified | Q9X5D0 | Chorismate |
| CHOR_112 | | | synthase | glutamicum | codon usage | | synthase (CS) |
| | | | | ATCC 13032 | for Cg and Sc | | |
| ScDD | 95.04 | P28777 | Chorismate | Saccharomyces | modified | P00924 | Enolase |
| CHOR_113 | | | synthase (CS) | cerevisiae | codon usage | | |
| | | | | S288c | for Cg and Sc | | |
| ScDD | 38.71 | Q9X5D1 | Shikimate kinase | Corynebacterium | modified | P32449 | DAHP synthase |
| CHOR_115 | | | (SK) | glutamicum | codon usage | | |
| | | | | ATCC 13032 | for Cg and Sc | | |
| ScDD | 21.18 | Q9X5D1 | Shikimate kinase | Corynebacterium | modified | Q8NRC0 | Shikimate 5- |
| CHOR_116 | | | (SK) | glutamicum | codon usage | | dehydrogenase |
| | | | | ATCC 13032 | for Cg and Sc | | |
| ScDD | 50.89 | Q9X5D1 | Shikimate kinase | Corynebacterium | modified | Q9X5D0 | Chorismate |
| CHOR_117 | | | (SK) | glutamicum | codon usage | | synthase (CS) |
| | | | | ATCC 13032 | for Cg and Sc | | |
| ScDD | 21.44 | Q9X5D1 | Shikimate kinase | Corynebacterium | modified | Q9X5D2 | 3-dehydroquinate |
| CHOR_118 | | | (SK) | glutamicum | codon usage | | synthase |
| | | | | ATCC 13032 | for Cg and Sc | | |
| ScDD | 19.97 | Q9X5D2 | 3-dehydroquinate | Corynebacterium | modified | Q8NRS1 | Enolase |
| CHOR_119 | | | synthase | glutamicum | codon usage | | |
| | | | | ATCC 13032 | for Cg and Sc | | |
| ScDD | 36.19 | Q9X5D1 | Shikimate kinase | Corynebacterium | modified | P32449 | DAHP synthase |
| CHOR_121 | | | (SK) | glutamicum | codon usage | | |
| | | | | ATCC 13032 | for Cg and Sc | | |
| ScDD | 26.74 | O52377 | 3-dehydroquinate | Corynebacterium | modified | | |
| CHOR_127 | | | dehydratase (3- | glutamicum | codon usage | | |
| | | | dehydroquinase) | ATCC 13032 | for Cg and Sc | | |
| ScDD | 26.72 | P32449 | Phospho-2- | Saccharomyces | modified | | |
| CHOR_129 | | | dehydro-3- | cerevisiae | codon usage | | |
| | | | deoxyheptonate | S288c | for Cg and Sc | | |
| | | | aldolase | | | | |
| ScDD | 20.23 | Q9X5D1 | Shikimate kinase | Corynebacterium | modified | Q8NQ64 | Transaldolase |
| CHOR_130 | | | (SK) | glutamicum | codon usage | | |
| | | | | ATCC 13032 | for Cg and Sc | | |
| ScDD | 46.30 | Q9X5D0 | Chorismate | Corynebacterium | modified | Q8NRS1 | Enolase |
| CHOR_131 | | | synthase (CS) | glutamicum | codon usage | | |
| | | | | ATCC 13032 | for Cg and Sc | | |

| | Enzyme 2 - | E2 Codon | E3 | | Enzyme 3 - | E3 Codon |
|---|---|---|---|---|---|---|
| Strain | source | Optimization | Uniprot | Enzyme 3 - | source | Optimization |
| Name | organism | Abbrev. | ID | activity name | organism | Abbrev. |
| CgDD | | | | | | |
| CHOR_20 | | | | | | |
| CgDD | | | | | | |
| CHOR_21 | | | | | | |
| CgDD | | | | | | |
| CHOR_25 | | | | | | |
| CgDD | | | | | | |
| CHOR_27 | | | | | | |
| ScDD | Corynebacterium | modified | O52377 | 3-dehydroquinate | Corynebacterium | modified |
| CHOR_24 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Corynebacterium | modified | O52377 | 3-dehydroquinate | Corynebacterium | modified |
| CHOR_25 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Corynebacterium | modified | Q9Z470 | 3-phosphoshikimate | Corynebacterium | modified |
| CHOR_27 | glutamicum | codon usage | | 1-carboxyvinyl- | glutamicum | codon |
| | ATCC 13032 | for Cg and Sc | | transferase | ATCC 13032 | usage for Cg and Sc |
| ScDD | Saccharomyces | Corynebacterium | Q8NRC0 | Shikimate 5- | Corynebacterium | modified |
| CHOR_28 | cerevisiae | glutamicum | | dehydrogenase | glutamicum | codon usage |
| | S288c | | | | ATCC 13032 | for Cg and Sc |
| ScDD | Corynebacterium | modified | P32449 | DAHP synthase | Saccharomyces | Corynebacterium |
| CHOR_29 | glutamicum | codon usage | | | cerevisiae | glutamicum |
| | ATCC 13032 | for Cg and Sc | | | S288c | |
| ScDD | Saccharomyces | Corynebacterium | O52377 | 3-dehydroquinate | Corynebacterium | modified |
| CHOR_30 | cerevisiae | glutamicum | | dehydratase (3- | glutamicum | codon usage |
| | S288c | | | dehydroquinase) | ATCC 13032 | for Cg and Sc |
| ScDD | Corynebacterium | modified | P32449 | DAHP synthase | Saccharomyces | Corynebacterium |
| CHOR_32 | glutamicum | codon usage | | | cerevisiae | glutamicum |
| | ATCC 13032 | for Cg and Sc | | | S288c | |
| ScDD | Saccharomyces | modified | | | | |
| CHOR_34 | cerevisiae | codon usage | | | | |
| | S288c | for Cg and Sc | | | | |
| ScDD | Saccharomyces | Corynebacterium | O52377 | 3-dehydroquinate | Corynebacterium | modified |
| CHOR_35 | cerevisiae | glutamicum | | dehydratase (3- | glutamicum | codon usage |
| | S288c | | | dehydroquinase) | ATCC 13032 | for Cg and Sc |
| ScDD | Corynebacterium | modified | O52377 | 3-dehydroquinate | Corynebacterium | modified |
| CHOR_37 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Corynebacterium | modified | O52377 | 3-dehydroquinate | Corynebacterium | modified |
| CHOR_40 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Corynebacterium | modified | Q8NQ64 | Transaldolase | Corynebacterium | modified |
| CHOR_41 | glutamicum | codon usage | | | glutamicum | codon usage |
| | ATCC 13032 | for Cg and Sc | | | ATCC 13032 | for Cg and Sc |
| ScDD | Corynebacterium | modified | O52377 | 3-dehydroquinate | Corynebacterium | modified |
| CHOR_43 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Saccharomyces | modified | P00924 | Enolase | Saccharomyces | modified |
| CHOR_45 | cerevisiae | codon usage | | | cerevisiae | codon usage |
| | S288c | for Cg and Sc | | | S288c | for Cg and Sc |
| ScDD | Saccharomyces | Corynebacterium | Q8NQ64 | Transaldolase | Corynebacterium | modified |
| CHOR_46 | cerevisiae | glutamicum | | | glutamicum | codon usage |
| | S288c | | | | ATCC 13032 | for Cg and Sc |
| ScDD | Saccharomyces | modified | P32449 | DAHP synthase | Saccharomyces | Corynebacterium |
| CHOR_47 | cerevisiae | codon usage | | | cerevisiae | glutamicum |
| | S288c | for Cg and Sc | | | S288c | |
| ScDD | | | | | | |
| CHOR_48 | | | | | | |
| ScDD | Saccharomyces | modified | P00924 | Enolase | Saccharomyces | modified |
| CHOR_49 | cerevisiae | codon usage | | | cerevisiae | codon usage |
| | S288c | for Cg and Sc | | | S288c | for Cg and Sc |
| ScDD | Corynebacterium | modified | Q8NRC0 | Shikimate 5- | Corynebacterium | modified |
| CHOR_52 | glutamicum | codon usage | | dehydrogenase | glutamicum | codon usage |
| | ATCC 13032 | for Cg and Sc | | | ATCC 13032 | for Cg and Sc |
| ScDD | Corynebacterium | modified | Q8NRC0 | Shikimate 5- | Corynebacterium | modified |
| CHOR_57 | glutamicum | codon usage | | dehydrogenase | glutamicum | codon usage |
| | ATCC 13032 | for Cg and Sc | | | ATCC 13032 | for Cg and Sc |
| ScDD | Corynebacterium | modified | Q9Z470 | 3-phosphoshikimate | Corynebacterium | modified |
| CHOR_58 | glutamicum | codon usage | | 1-carboxyvinyl- | glutamicum | codon usage |
| | ATCC 13032 | for Cg and Sc | | transferase | ATCC 13032 | for Cg and Sc |
| ScDD | Corynebacterium | modified | O52377 | 3-dehydroquinate | Corynebacterium | modified |
| CHOR_61 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Corynebacterium | modified | Q8NRS1 | Enolase | Corynebacterium | modified |
| CHOR_63 | glutamicum | codon usage | | | glutamicum | codon usage |
| | ATCC 13032 | for Cg and Sc | | | ATCC 13032 | for Cg and Sc |
| ScDD | Corynebacterium | modified | Q9Z470 | 3-phosphoshikimate | Corynebacterium | modified |
| CHOR_64 | glutamicum | codon usage | | 1-carboxyvinyl- | glutamicum | codon usage |
| | ATCC 13032 | for Cg and Sc | | transferase | ATCC 13032 | for Cg and Sc |
| ScDD | Saccharomyces | Corynebacterium | Q8NQ64 | Transaldolase | Corynebacterium | modified |
| CHOR_65 | cerevisiae | glutamicum | | | glutamicum | codon usage |
| | S288c | | | | ATCC 13032 | for Cg and Sc |
| ScDD | Corynebacterium | modified | Q9Z470 | 3-phosphoshikimate | Corynebacterium | modified |
| CHOR_69 | glutamicum | codon usage | | 1-carboxyvinyl- | glutamicum | codon usage |
| | ATCC 13032 | for Cg and Sc | | transferase | ATCC 13032 | for Cg and Sc |
| ScDD | | | | | | |
| CHOR_70 | | | | | | |
| ScDD | Corynebacterium | modified | Q9Z470 | 3-phosphoshikimate | Corynebacterium | modified |
| CHOR_72 | glutamicum | codon usage | | 1-carboxyvinyl- | glutamicum | codon usage |
| | ATCC 13032 | for Cg and Sc | | transferase | ATCC 13032 | for Cg and Sc |
| ScDD | Corynebacterium | modified | Q9Z470 | 3-phosphoshikimate | Corynebacterium | modified |
| CHOR_73 | glutamicum | codon usage | | 1-carboxyvinyl- | glutamicum | codon usage |
| | ATCC 13032 | for Cg and Sc | | transferase | ATCC 13032 | for Cg and Sc |
| ScDD | Corynebacterium | modified | O52377 | 3-dehydroquinate | Corynebacterium | modified |
| CHOR_75 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Corynebacterium | modified | O52377 | 3-dehydroquinate | Corynebacterium | modified |
| CHOR_76 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Corynebacterium | modified | Q8NRS1 | Enolase | Corynebacterium | modified |
| CHOR_77 | glutamicum | codon usage | | | glutamicum | codon usage |
| | ATCC 13032 | for Cg and Sc | | | ATCC 13032 | for Cg and Sc |
| ScDD | Corynebacterium | modified | O52377 | 3-dehydroquinate | Corynebacterium | modified |
| CHOR_78 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Saccharomyces | Corynebacterium | Q9Z470 | 3-phosphoshikimate | Corynebacterium | modified |
| CHOR_79 | cerevisiae | glutamicum | | 1-carboxyvinyl- | glutamicum | codon usage |
| | S288c | | | transferase | ATCC 13032 | for Cg and Sc |
| ScDD | Corynebacterium | modified | Q9Z470 | 3-phosphoshikimate | Corynebacterium | modified |
| CHOR_80 | glutamicum | codon usage | | 1-carboxyvinyl- | glutamicum | codon usage |
| | ATCC 13032 | for Cg and Sc | | transferase | ATCC 13032 | for Cg and Sc |
| ScDD | Corynebacterium | modified | Q9Z470 | 3-phosphoshikimate | Corynebacterium | modified |
| CHOR_82 | glutamicum | codon usage | | 1-carboxyvinyl- | glutamicum | codon usage |
| | ATCC 13032 | for Cg and Sc | | transferase | ATCC 13032 | for Cg and Sc |
| ScDD | Corynebacterium | modified | Q8NRC0 | Shikimate 5- | Corynebacterium | modified |
| CHOR_84 | glutamicum | codon usage | | dehydrogenase | glutamicum | codon usage |
| | ATCC 13032 | for Cg and Sc | | | ATCC 13032 | for Cg and Sc |
| ScDD | Saccharomyces | modified | P28777 | Chorismate | Saccharomyces | modified |
| CHOR_86 | cerevisiae | codon usage | | synthase (CS) | cerevisiae | codon usage |
| | S288c | for Cg and Sc | | | S288c | for Cg and Sc |
| ScDD | Saccharomyces | Corynebacterium | | | | |
| CHOR_87 | cerevisiae | glutamicum | | | | |
| | S288c | | | | | |
| ScDD | Saccharomyces | Corynebacterium | | | | |
| CHOR_88 | cerevisiae | glutamicum | | | | |
| | S288c | | | | | |
| ScDD | Saccharomyces | modified | | | | |
| CHOR_89 | cerevisiae | codon usage | | | | |
| | S288c | for Cg and Sc | | | | |
| ScDD | Corynebacterium | modified | | | | |
| CHOR_90 | glutamicum | codon usage | | | | |
| | ATCC 13032 | for Cg and Sc | | | | |
| ScDD | Corynebacterium | modified | | | | |
| CHOR_91 | glutamicum | codon usage | | | | |
| | ATCC 13032 | for Cg and Sc | | | | |
| ScDD | Corynebacterium | modified | | | | |
| CHOR_92 | glutamicum | codon usage | | | | |
| | ATCC 13032 | for Cg and Sc | | | | |
| ScDD | Corynebacterium | modified | | | | |
| CHOR_93 | glutamicum | codon usage | | | | |
| | ATCC 13032 | for Cg and Sc | | | | |
| ScDD | Corynebacterium | modified | | | | |
| CHOR_96 | glutamicum | codon usage | | | | |
| | ATCC 13032 | for Cg and Sc | | | | |
| ScDD | Corynebacterium | modified | | | | |
| CHOR_97 | glutamicum | codon usage | | | | |
| | ATCC 13032 | for Cg and Sc | | | | |
| ScDD | Saccharomyces | modified | | | | |
| CHOR_99 | cerevisiae | codon usage | | | | |
| | S288c | for Cg and Sc | | | | |
| ScDD | Saccharomyces | Corynebacterium | | | | |
| CHOR_101 | cerevisiae | glutamicum | | | | |
| | S288c | | | | | |
| ScDD | Saccharomyces | Corynebacterium | | | | |
| CHOR_102 | cerevisiae | glutamicum | | | | |
| | S288c | | | | | |
| ScDD | Saccharomyces | modified | P32449 | DAHP synthase | Saccharomyces | Corynebacterium |
| CHOR_103 | cerevisiae | codon usage | | | cerevisiae | glutamicum |
| | S288c | for Cg and Sc | | | S288c | |
| ScDD | Saccharomyces | Corynebacterium | Q8NQ64 | Transaldolase | Corynebacterium | modified |
| CHOR_104 | cerevisiae | glutamicum | | | glutamicum | codon usage |
| | S288c | | | | ATCC 13032 | for Cg and Sc |
| ScDD | Corynebacterium | modified | Q8NRC0 | Shikimate 5- | Corynebacterium | modified |
| CHOR_106 | glutamicum | codon usage | | dehydrogenase | glutamicum | codon usage |
| | ATCC 13032 | for Cg and Sc | | | ATCC 13032 | for Cg and Sc |
| ScDD | Corynebacterium | modified | Q9Z470 | 3-phosphoshikimate | Corynebacterium | modified |
| CHOR_107 | glutamicum | codon usage | | 1-carboxyvinyl- | glutamicum | codon usage |
| | ATCC 13032 | for Cg and Sc | | transferase | ATCC 13032 | for Cg and Sc |
| ScDD | Corynebacterium | modified | O52377 | 3-dehydroquinate | Corynebacterium | modified |
| CHOR_109 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Saccharomyces | Corynebacterium | Q8NRC0 | Shikimate 5- | Corynebacterium | modified |
| CHOR_110 | cerevisiae | glutamicum | | dehydrogenase | glutamicum | codon usage |
| | S288c | | | | ATCC 13032 | for Cg and Sc |
| ScDD | Corynebacterium | modified | Q8NQ64 | Transaldolase | Corynebacterium | modified |
| CHOR_112 | glutamicum | codon usage | | | glutamicum | codon usage |
| | ATCC 13032 | for Cg and Sc | | | ATCC 13032 | for Cg and Sc |
| ScDD | Saccharomyces | modified | P32449 | DAHP synthase | Saccharomyces | Corynebacterium |
| CHOR_113 | cerevisiae | codon usage | | | cerevisiae | glutamicum |
| | S288c | for Cg and Sc | | | S288c | |
| ScDD | Saccharomyces | Corynebacterium | Q9Z470 | 3-phosphoshikimate | Corynebacterium | modified |
| CHOR_115 | cerevisiae | glutamicum | | 1-carboxyvinyl- | glutamicum | codon usage |
| | S288c | | | transferase | ATCC 13032 | for Cg and Sc |
| ScDD | Corynebacterium | modified | O52377 | 3-dehydroquinate | Corynebacterium | modified |
| CHOR_116 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Corynebacterium | modified | P32449 | DAHP synthase | Saccharomyces | Corynebacterium |
| CHOR_117 | glutamicum | codon usage | | | cerevisiae | glutamicum |
| | ATCC 13032 | for Cg and Sc | | | S288c | |
| ScDD | Corynebacterium | modified | Q9X5D0 | Chorismate | Corynebacterium | modified |
| CHOR_118 | glutamicum | codon usage | | synthase (CS) | glutamicum | codon usage |
| | ATCC 13032 | for Cg and Sc | | | ATCC 13032 | for Cg and Sc |
| ScDD | Corynebacterium | modified | O52377 | 3-dehydroquinate | Corynebacterium | modified |
| CHOR_119 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Saccharomyces | Corynebacterium | O52377 | 3-dehydroquinate | Corynebacterium | modified |
| CHOR_121 | cerevisiae | glutamicum | | dehydratase (3- | glutamicum | codon usage |
| | S288c | | | dehydroquinase) | ATCC 13032 | for Cg and Sc |
| ScDD | | | | | | |
| CHOR_127 | | | | | | |
| ScDD | | | | | | |
| CHOR_129 | | | | | | |
| ScDD | Corynebacterium | modified | O52377 | 3-dehydroquinate | Corynebacterium | modified |
| CHOR_130 | glutamicum | codon usage | | dehydratase (3- | glutamicum | codon usage |
| | ATCC 13032 | for Cg and Sc | | dehydroquinase) | ATCC 13032 | for Cg and Sc |
| ScDD | Corynebacterium | modified | P32449 | DAHP synthase | Saccharomyces | Corynebacterium |
| CHOR_131 | glutamicum | codon usage | | | cerevisiae | glutamicum |
| | ATCC 13032 | for Cg and Sc | | | S288c | |

TABLE 3
Third-Round Results
In addition to the enzymes in this table, the Corynebacterium glutamicum strains contain two
copies of chorismate dehydratase (UniProt ID B1W536), and the Saccharomyces cerevisiae strains
contain chorismate dehydratase (UniProt C6J436), shikimate kinase (UniProt ID Q9X5D1), 3-dehydroquinate
synthase (UniProt ID Q9X5D2), and DAHP synthase (UniProt ID P32449), containing the amino acid
substitution K229L, which were the best enzymes in each best-performing host from the second
round of genetic engineering(see Tables 1 and 2). All of theDAHP synthases (UniProt ID P32449)
tested in the third round contained K229L, which reduces pathway feedback inhibition.

| | E1 | | E1 - | Enzyme 1 - | E1 Codon | E2 | | E2 - |
|---|---|---|---|---|---|---|---|---|
| Strain | Uniprot | Enzyme 1 - | Modifi- | source | Optimization | Uniprot | Enzyme 2 - | Modifi- |
| Name | ID | activity name | cations | organism | Abbrev. | ID | activity name | cations |
| Corynebacterium glutamicum | | | | | | | | |
| CgDD | Q9X5D1 | Shikimate | | Corynebacterium | modified | O52377 | 3-dehydroquinate | |
| CHOR_28 | | kinase (SK) | | glutamicum | codon usage | | dehydratase | |
| | | | | ATCC 13032 | for Cg and Sc | | (3-dehydroquinase) | |
| CgDD | Q9X5D1 | Shikimate | | Corynebacterium | modified | O52377 | 3-dehydroquinate | |
| CHOR_30 | | kinase (SK) | | glutamicum | codon usage | | dehydratase | |
| | | | | ATCC 13032 | for Cg and Sc | | (3-dehydroquinase) | |
| CgDD | B1W536 | chorismate | | Streptomyces | modified | | | |
| CHOR_31 | | dehydratase | | griseus subsp. | codon usage | | | |
| | | | | griseus | for Cg and Sc | | | |
| | | | | JCM 4626 | | | | |
| CgDD | Q9X5D1 | Shikimate | | Corynebacterium | modified | Q8NRS1 | Enolase | |
| CHOR_33 | | kinase (SK) | | glutamicum | codon usage | | | |
| | | | | ATCC 13032 | for Cg and Sc | | | |
| CgDD | Q9Z470 | 3-phosphoshikimate | | Corynebacterium | modified | Q9X5D0 | Chorismate | |
| CHOR_34 | | 1-carboxyvinyl- | | glutamicum | codon usage | | synthase (CS) | |
| | | transferase | | ATCC 13032 | for Cg and Sc | | | |
| CgDD | Q9X5D1 | Shikimate | | Corynebacterium | modified | O52377 | 3-dehydroquinate | |
| CHOR_35 | | kinase (SK) | | glutamicum | codon usage | | dehydratase | |
| | | | | ATCC 13032 | for Cg and Sc | | (3-dehydroquinase) | |
| CgDD | P00888 | DAHP | N8K | Escherichia | modified | | | |
| CHOR_36 | | synthase | | coli K12 | codon usage | | | |
| | | | | | for Cg and Sc | | | |
| CgDD | P0AB91 | DAHP | P150L | Escherichia | modified | | | |
| CHOR_37 | | synthase | | coli K12 | codon usage | | | |
| | | | | | for Cg and Sc | | | |
| CgDD | A0A0F7VYE2 | chorismate | | Streptomyces | modified | | | |
| CHOR_38 | | dehydratase | | leeuwenhoekii | codon usage | | | |
| | | | | | for Cg and Sc | | | |
| CgDD | O52377 | 3-dehydroquinate | | Corynebacterium | modified | Q9Z470 | 3-phosphoshikimate | |
| CHOR_39 | | dehydratase | | glutamicum | codon usage | | 1-carboxyvinyl- | |
| | | (3-dehydroquinase) | | ATCC 13032 | for Cg and Sc | | transferase | |
| CgDD | O52377 | 3-dehydroquinate | | Corynebacterium | modified | Q8NRC0 | Shikimate 5- | |
| CHOR_40 | | dehydratase | | glutamicum | codon usage | | dehydrogenase | |
| | | (3-dehydroquinase) | | ATCC 13032 | for Cg and Sc | | | |
| CgDD | Q9Z470 | 3-phosphoshikimate | | Corynebacterium | modified | Q8NRC0 | Shikimate 5- | |
| CHOR_41 | | 1-carboxyvinyl- | | glutamicum | codon usage | | dehydrogenase | |
| | | transferase | | ATCC 13032 | for Cg and Sc | | | |
| CgDD | B1W536 | chorismate | | Streptomyces | modified | Q8NRS1 | Enolase | |
| CHOR_42 | | dehydratase | | griseus subsp. | codon usage | | | |
| | | | | griseus | for Cg and Sc | | | |
| | | | | JCM 4626 | | | | |
| CgDD | O52377 | 3-dehydroquinate | | Corynebacterium | modified | Q9X5D2 | 3-dehydroquinate | |
| CHOR_43 | | dehydratase | | glutamicum | codon usage | | synthase | |
| | | (3-dehydroquinase) | | ATCC 13032 | for Cg and Sc | | | |
| CgDD | Q9Z470 | 3-phosphoshikimate | | Corynebacterium | modified | Q9X5D0 | Chorismate | |
| CHOR_44 | | 1-carboxyvinyl- | | glutamicum | codon usage | | synthase (CS) | |
| | | transferase | | ATCC 13032 | for Cg and Sc | | | |
| CgDD | S5V7C6 | chorismate | | Streptomyces | modified | | | |
| CHOR_45 | | dehydratase | | collinus | codon usage | | | |
| | | | | DSM 40733 | for Cg and Sc | | | |
| CgDD | Q9Z470 | 3-phosphoshikimate | | Corynebacterium | modified | Q8NQ64 | Transaldolase | |
| CHOR_48 | | 1-carboxyvinyl- | | glutamicum | codon usage | | | |
| | | transferase | | ATCC 13032 | for Cg and Sc | | | |
| Saccharomyces cerevisiae | | | | | | | | |
| ScDD | Q9X5D2 | 3-dehydroquinate | | Corynebacterium | modified | O52377 | 3-dehydroquinate | |
| CHOR_133 | | synthase | | glutamicum | codon usage | | dehydratase | |
| | | | | ATCC 13032 | for Cg and Sc | | (3-dehydroquinase) | |
| ScDD | Q8NQI2 | 6-phospho- | S361F | Corynebacterium | modified | | | |
| CHOR_135 | | gluconate | | glutamicum | codon usage | | | |
| | | dehydrogenase | | ATCC 13032 | for Cg and Sc | | | |
| ScDD | C6XW11 | chorismate | | Pedobacter | modified | | | |
| CHOR_136 | | dehydratase | | heparinus | codon usage | | | |
| | | | | ATCC 13125 | for Cg and Sc | | | |
| ScDD | O52377 | 3-dehydroquinate | | Corynebacterium | modified | A4QEF2 | Glucose-6- | A243T |
| CHOR_137 | | dehydratase | | glutamicum | codon usage | | phosphate | |
| | | (3-dehydroquinase) | | ATCC 13032 | for Cg and Sc | | dehydrogenase | |
| ScDD | P35170 | Phospho-2- | | Corynebacterium | modified | | | |
| CHOR_138 | | dehydro-3- | | glutamicum | codon usage | | | |
| | | deoxyheptonate | | ATCC 13032 | for Cg and Sc | | | |
| | | aldolase | | | | | | |
| ScDD | P0AB91 | DAHP | D146N | Escherichia | modified | | | |
| CHOR_139 | | synthase | | coli K12 | codon usage | | | |
| | | | | | for Cg and Sc | | | |
| ScDD | Q8NQI2 | 6-phospho- | S361F | Corynebacterium | modified | Q8NQ64 | Transaldolase | |
| CHOR_140 | | gluconate | | glutamicum | codon usage | | | |
| | | dehydrogenase | | ATCC 13032 | for Cg and Sc | | | |
| ScDD | Q8NRS1 | Enolase | | Corynebacterium | modified | Q8NQI2 | 6-phospho- | S361F |
| CHOR_141 | | | | glutamicum | codon usage | | gluconate | |
| | | | | ATCC 13032 | for Cg and Sc | | dehydrogenase | |
| ScDD | Q8NRS1 | Enolase | | Corynebacterium | modified | A4QEF2 | Glucose-6- | A243T |
| CHOR_142 | | | | glutamicum | codon usage | | phosphate | |
| | | | | ATCC 13032 | for Cg and Sc | | dehydrogenase | |
| ScDD | Q8NRS1 | Enolase | | Corynebacterium | modified | A4QEF2 | Glucose-6- | A243T |
| CHOR_143 | | | | glutamicum | codon usage | | phosphate | |
| | | | | ATCC 13032 | for Cg and Sc | | dehydrogenase | |
| ScDD | Q9X5D2 | 3-dehydroquinate | | Corynebacterium | modified | Q9Z470 | 3-phosphoshikimate | |
| CHOR_144 | | synthase | | glutamicum | codon usage | | 1-carboxyvinyl- | |
| | | | | ATCC 13032 | for Cg and Sc | | transferase | |
| ScDD | O52377 | 3-dehydroquinate | | Corynebacterium | modified | Q8NQI2 | 6-phospho- | S361F |
| CHOR_145 | | dehydratase | | glutamicum | codon usage | | gluconate | |
| | | (3-dehydroquinase) | | ATCC 13032 | for Cg and Sc | | dehydrogenase | |
| ScDD | Q9X5D1 | Shikimate | | Corynebacterium | modified | A4QEF2 | Glucose-6- | A243T |
| CHOR_146 | | kinase (SK) | | glutamicum | codon usage | | phosphate | |
| | | | | ATCC 13032 | for Cg and Sc | | dehydrogenase | |
| ScDD | C6CUC4 | chorismate | | Paenibacillus | modified | | | |
| CHOR_147 | | dehydratase | | sp. strain | codon usage | | | |
| | | | | JDR-2 | for Cg and Sc | | | |
| ScDD | O52377 | 3-dehydroquinate | | Corynebacterium | modified | A4QEF2 | Glucose-6- | A243T |
| CHOR_148 | | dehydratase | | glutamicum | codon usage | | phosphate | |
| | | (3-dehydroquinase) | | ATCC 13032 | for Cg and Sc | | dehydrogenase | |
| ScDD | Q9X5D2 | 3-dehydroquinate | | Corynebacterium | modified | O52377 | 3-dehydroquinate | |
| CHOR_149 | | synthase | | glutamicum | codon usage | | dehydratase | |
| | | | | ATCC 13032 | for Cg and Sc | | (3-dehydroquinase) | |
| ScDD | O52377 | 3-dehydroquinate | | Corynebacterium | modified | Q8NQ64 | Transaldolase | |
| CHOR_150 | | dehydratase | | glutamicum | codon usage | | | |
| | | (3-dehydroquinase) | | ATCC 13032 | for Cg and Sc | | | |
| ScDD | Q9X5D1 | Shikimate | | Corynebacterium | modified | Q9X5D0 | Chorismate | |
| CHOR_151 | | kinase (SK) | | glutamicum | codon usage | | synthase (CS) | |
| | | | | ATCC 13032 | for Cg and Sc | | | |

| | Enzyme 2 - | E2 Codon | E3 | Enzyme 3 - | E3 - | Enzyme 3 - | E3 Codon |
|---|---|---|---|---|---|---|---|
| Strain | source | Optimization | Uniprot | activity | Modifi- | source | Optimization |
| Name | organism | Abbrev. | ID | name | cations | organism | Abbrev |
| Corynebacterium | | | | | | | |
| glutamicum | | | | | | | |
| CgDD | Corynebacterium | modified | A4QEF2 | Glucose-6- | A243T | Corynebacterium | modified |
| CHOR_28 | glutamicum | codon usage | | phosphate | | glutamicum | codon usage |
| | strain | for Cg and Sc | | dehydrogenase | | (strain R) | for Cg and Sc |
| | ATCC 13032 | | | | | | |
| CgDD | Corynebacterium | modified | Q9X5D2 | 3-dehydroquinate | | Corynebacterium | modified |
| CHOR_30 | glutamicum | codon usage | | synthase | | glutamicum | codon usage |
| | strain | for Cg and Sc | | | | ATCC 13032 | for Cg and Sc |
| | ATCC 13032 | | | | | | |
| CgDD | | | | | | | |
| CHOR_31 | | | | | | | |
| CgDD | Corynebacterium | modified | O52377 | 3-dehydroquinate | | Corynebacterium | modified |
| CHOR_33 | glutamicum | codon usage | | dehydratase | | glutamicum | codon usage |
| | strain | for Cg and Sc | | (3-dehydroquinase) | | ATCC 13032 | for Cg and Sc |
| | ATCC 13032 | | | | | | |
| CgDD | Corynebacterium | modified | Q8NRC0 | Shikimate 5- | | Corynebacterium | modified |
| CHOR_34 | glutamicum | codon usage | | dehydrogenase | | glutamicum | codon usage |
| | strain | for Cg and Sc | | | | ATCC 13032 | for Cg and Sc |
| | ATCC 13032 | | | | | | |
| CgDD | Corynebacterium | modified | Q8NRC0 | Shikimate 5- | | Corynebacterium | modified |
| CHOR_35 | glutamicum | codon usage | | dehydrogenase | | glutamicum | codon usage |
| | strain | for Cg and Sc | | | | ATCC 13032 | for Cg and Sc |
| | ATCC 13032 | | | | | | |
| CgDD | | | | | | | |
| CHOR_36 | | | | | | | |
| CgDD | | | | | | | |
| CHOR_37 | | | | | | | |
| CgDD | | | | | | | |
| CHOR_38 | | | | | | | |
| CgDD | Corynebacterium | modified | | | | | |
| CHOR_39 | glutamicum | codon usage | | | | | |
| | strain | for Cg and Sc | | | | | |
| | ATCC 13032 | | | | | | |
| CgDD | Corynebacterium | modified | | | | | |
| CHOR_40 | glutamicum | codon usage | | | | | |
| | strain | for Cg and Sc | | | | | |
| | ATCC 13032 | | | | | | |
| CgDD | Corynebacterium | modified | Q8NQ64 | Transaldolase | | Corynebacterium | modified |
| CHOR_41 | glutamicum | codon usage | | | | glutamicum | codon usage |
| | strain | for Cg and Sc | | | | ATCC 13032 | for Cg and Sc |
| | ATCC 13032 | | | | | | |
| CgDD | Corynebacterium | modified | Q9Z470 | 3-phosphoshikimate | | Corynebacterium | modified |
| CHOR_42 | glutamicum | codon usage | | 1-carboxyvinyl- | | glutamicum | codon usage |
| | strain | for Cg and Sc | | transferase | | ATCC 13032 | for Cg and Sc |
| | ATCC 13032 | | | | | | |
| CgDD | Corynebacterium | modified | | | | | |
| CHOR_43 | glutamicum | codon usage | | | | | |
| | strain | for Cg and Sc | | | | | |
| | ATCC 13032 | | | | | | |
| CgDD | Corynebacterium | modified | | | | | |
| CHOR_44 | glutamicum | codon usage | | | | | |
| | strain | for Cg and Sc | | | | | |
| | ATCC 13032 | | | | | | |
| CgDD | | | | | | | |
| CHOR_45 | | | | | | | |
| CgDD | Corynebacterium | modified | | | | | |
| CHOR_48 | glutamicum | codon usage | | | | | |
| | strain | for Cg and Sc | | | | | |
| | ATCC 13032 | | | | | | |
| Saccharomyces | | | | | | | |
| cerevisiae | | | | | | | |
| ScDD | Corynebacterium | modified | A4QEF2 | Glucose-6- | A243T | Corynebacterium | modified |
| CHOR_133 | glutamicum | codon usage | | phosphate | | glutamicum | codon usage |
| | strain | for Cg and Sc | | dehydrogenase | | (strain R) | for Cg and Sc |
| | ATCC 13032 | | | | | | |
| ScDD | | | | | | | |
| CHOR_135 | | | | | | | |
| ScDD | | | | | | | |
| CHOR_136 | | | | | | | |
| ScDD | Corynebacterium | modified | Q8NQ64 | Transaldolase | | Corynebacterium | modified |
| CHOR_137 | glutamicum | codon usage | | | | glutamicum | codon usage |
| | (strain R) | for Cg and Sc | | | | ATCC 13032 | for Cg and Sc |
| ScDD | | | | | | | |
| CHOR_138 | | | | | | | |
| ScDD | | | | | | | |
| CHOR_139 | | | | | | | |
| ScDD | Corynebacterium | modified | | | | | |
| CHOR_140 | glutamicum | codon usage | | | | | |
| | strain | for Cg and Sc | | | | | |
| | ATCC 13032 | | | | | | |
| ScDD | Corynebacterium | modified | | | | | |
| CHOR_141 | glutamicum | codon usage | | | | | |
| | strain | for Cg and Sc | | | | | |
| | ATCC 13032 | | | | | | |
| ScDD | Corynebacterium | modified | Q8NQI2 | 6-phospho- | S361F | Corynebacterium | modified |
| CHOR_142 | glutamicum | codon usage | | gluconate | | glutamicum | codon usage |
| | (strain R) | for Cg and Sc | | dehydrogenase | | ATCC 13032 | for Cg and Sc |
| ScDD | Corynebacterium | modified | A4QEF2 | Glucose-6- | A243T | Corynebacterium | modified |
| CHOR_143 | glutamicum | codon usage | | phosphate | | glutamicum | codon usage |
| | (strain R) | for Cg and Sc | | dehydrogenase | | (strain R) | for Cg and Sc |
| ScDD | Corynebacterium | modified | Q8NQI2 | 6-phospho- | S361F | Corynebacterium | modified |
| CHOR_144 | glutamicum | codon usage | | gluconate | | glutamicum | codon usage |
| | strain | for Cg and Sc | | dehydrogenase | | ATCC 13032 | for Cg and Sc |
| | ATCC 13032 | | | | | | |
| ScDD | Corynebacterium | modified | | | | | |
| CHOR_145 | glutamicum | codon usage | | | | | |
| | strain | for Cg and Sc | | | | | |
| | ATCC 13032 | | | | | | |
| ScDD | Corynebacterium | modified | Q8NRC0 | Shikimate 5- | | Corynebacterium | modified |
| CHOR_146 | glutamicum | codon usage | | dehydrogenase | | glutamicum | codon usage |
| | (strain R) | for Cg and Sc | | | | ATCC 13032 | for Cg and Sc |
| ScDD | | | | | | | |
| CHOR_147 | | | | | | | |
| ScDD | Corynebacterium | modified | | | | | |
| CHOR_148 | glutamicum | codon usage | | | | | |
| | (strain R) | for Cg and Sc | | | | | |
| ScDD | Corynebacterium | modified | | | | | |
| CHOR_149 | glutamicum | codon usage | | | | | |
| | strain | for Cg and Sc | | | | | |
| | ATCC 13032 | | | | | | |
| ScDD | Corynebacterium | modified | | | | | |
| CHOR_150 | glutamicum | codon usage | | | | | |
| | strain | for Cg and Sc | | | | | |
| | ATCC 13032 | | | | | | |
| ScDD | Corynebacterium | modified | Q8NQ64 | Transaldolase | | Corynebacterium | modified |
| CHOR_151 | glutamicum | codon usage | | | | glutamicum | codon usage |
| | strain | for Cg and Sc | | | | ATCC 13032 | for Cg and Sc |
| | ATCC 13032 | | | | | | |

TABLE 4
Fourth-Round Results
In addition to the enzymes in this table, the Corynebacterium glutamicum strains contain two
copies of chorismate dehydratase from Streptomyces griseus (UniProt ID B1W536) and a feedback-
deregulated variant of an Escherichia coli K12 DAHP synthase (UniProt ID POAB91) including P150L.

| | Titer | E1 | | | E2 | |
|---|---|---|---|---|---|---|
| strain_name | μg/L | Uniprot | E1 Name | E1 Source | Uniprot | E2 Name |
| CgDD | 449043.4 | B1W536 | Chorismate | Streptomyces | P32449 | Phospho-2- |
| CHOR_49 | | | dehydratase | griseus subsp. | | dehydro-3- |
| | | | | griseus (strain | | deoxyheptonate |
| | | | | JCM 4626/ | | aldolase, |
| | | | | NBRC 13350) | | tyrosine- |
| | | | | | | inhibited |
| CgDD | 461256.3 | P32449 | Phospho-2- | Saccharomyces | P08566 | Pentafunctional |
| CHOR_50 | | | dehydro-3- | cerevisiae | | AROM |
| | | | deoxyheptonate | (strain ATCC | | polypeptide |
| | | | aldolase, | 204508/S288c) | | [Includes: 3- |
| | | | tyrosine- | (Baker's yeast) | | dehydroquinate |
| | | | inhibited | | | synthase |
| CgDD | 275256.2 | P0AB91 | Phospho-2- | Escherichia | P27302 | Transketolase 1 |
| CHOR_51 | | | dehydro-3- | coli (strain | | |
| | | | deoxyheptonate | K12) | | |
| | | | aldolase, Phe- | | | |
| | | | sensitive | | | |
| CgDD | 376100 | P27302 | Transketolase 1 | Escherichia | P0AB91 | Phospho-2- |
| CHOR_52 | | | | coli (strain | | dehydro-3- |
| | | | | K12) | | deoxyheptonate |
| | | | | | | aldolase, Phe- |
| | | | | | | sensitive |
| CgDD | 451448.3 | B1W536 | Chorismate | Streptomyces | P32449 | Phospho-2- |
| CHOR_54 | | | dehydratase | griseus subsp. | | dehydro-3- |
| | | | | griseus (strain | | deoxyheptonate |
| | | | | JCM 4626/ | | aldolase, |
| | | | | NBRC 13350) | | tyrosine- |
| | | | | | | inhibited |
| CgDD | 239355 | Q9ZMU5 | Phospho-2- | Helicobacter | P27302 | Transketolase 1 |
| CHOR_55 | | | dehydro-3- | pylori (strain | | |
| | | | deoxyheptonate | J99/ATCC | | |
| | | | aldolase | 700824) | | |
| | | | | (Campylobacter | | |
| | | | | pylori J99) | | |
| CgDD | 459730 | P32449 | Phospho-2- | Saccharomyces | A0A087KDJ2 | Chorismate |
| CHOR_58 | | | dehydro-3- | cerevisiae | | dehydratase |
| | | | deoxyheptonate | (strain ATCC | | |
| | | | aldolase, | 204508/S288c) | | |
| | | | tyrosine- | (Baker's yeast) | | |
| | | | inhibited | | | |
| CgDD | 344151.8 | B1W536 | Chorismate | Streptomyces | P05194 | 3-dehydroquinate |
| CHOR_59 | | | dehydratase | griseus subsp. | | dehydratase |
| | | | | griseus (strain | | |
| | | | | JCM 4626/ | | |
| | | | | NBRC 13350) | | |
| CgDD | 433450.9 | Q9X5C9 | Quinate/ | Corynebacterium | B1W536 | Chorismate |
| CHOR_60 | | | shikimate | glutamicum | | dehydratase |
| | | | dehydrogenase | (strain ATCC | | |
| | | | | 13032/DSM | | |
| | | | | 20300/JCM | | |
| | | | | 1318/LMG | | |
| | | | | 3730/NCIMB | | |
| | | | | 10025) | | |
| CgDD | 463206.4 | P32449 | Phospho-2- | Saccharomyces | P0A6D3 | 3-phosphoshikimate |
| CHOR_61 | | | dehydro-3- | cerevisiae | | 1-carboxyvinyl- |
| | | | deoxyheptonate | (strain ATCC | | transferase |
| | | | aldolase, | 204508/S288c) | | |
| | | | tyrosine- | (Baker's yeast) | | |
| | | | inhibited | | | |
| CgDD | 131794.6 | B1W536 | Chorismate | Streptomyces | Q01651 | Glyceraldehyde- |
| CHOR_62 | | | dehydratase | griseus subsp. | | 3-phosphate |
| | | | | griseus (strain | | dehydrogenase |
| | | | | JCM 4626/ | | |
| | | | | NBRC 13350) | | |
| CgDD | 42072.47 | Q9X5D0 | Chorismate | Corynebacterium | P08566 | Pentafunctional |
| CHOR_64 | | | synthase | glutamicum | | AROM |
| | | | | (strain ATCC | | polypeptide |
| | | | | 13032/DSM | | [Includes: 3- |
| | | | | 20300/JCM | | dehydroquinate |
| | | | | 1318/LMG | | synthase |
| | | | | 3730/NCIMB | | |
| | | | | 10025) | | |
| CgDD | 238765.7 | P27302 | Transketolase 1 | Escherichia | Q9WYH8 | Phospho-2- |
| CHOR_65 | | | | coli (strain | | dehydro-3- |
| | | | | K12) | | deoxyheptonate |
| | | | | | | aldolase |
| CgDD | 353822 | Q9X5D0 | Chorismate | Corynebacterium | Q8NRC0 | Shikimate 5- |
| CHOR_66 | | | synthase | glutamicum | | dehydrogenase |
| | | | | (strain ATCC | | |
| | | | | 13032/DSM | | |
| | | | | 20300/JCM | | |
| | | | | 1318/LMG | | |
| | | | | 3730/NCIMB | | |
| | | | | 10025) | | |
| CgDD | 352275.6 | Q9X5D0 | Chorismate | Corynebacterium | Q9Z470 | 3-phosphoshikimate |
| CHOR_67 | | | synthase | glutamicum | | 1-carboxyvinyl- |
| | | | | (strain ATCC | | transferase |
| | | | | 13032/DSM | | |
| | | | | 20300/JCM | | |
| | | | | 1318/LMG | | |
| | | | | 3730/NCIMB | | |
| | | | | 10025) | | |
| CgDD | 340481 | Q9X5D2 | 3-dehydroquinate | Corynebacterium | O52377 | 3-dehydroquinate |
| CHOR_68 | | | synthase | glutamicum | | dehydratase |
| | | | | (strain ATCC | | |
| | | | | 13032/DSM | | |
| | | | | 20300/JCM | | |
| | | | | 1318/LMG | | |
| | | | | 3730/NCIMB | | |
| | | | | 10025) | | |
| CgDD | 327425.1 | O52377 | 3-dehydroquinate | Corynebacterium | Q9X5D2 | 3-dehydroquinate |
| CHOR_69 | | | dehydratase | glutamicum | | synthase |
| | | | | (strain ATCC | | |
| | | | | 13032/DSM | | |
| | | | | 20300/JCM | | |
| | | | | 1318/LMG | | |
| | | | | 3730/NCIMB | | |
| | | | | 10025) | | |
| CgDD | 363975.8 | O52377 | 3-dehydroquinate | Corynebacterium | Q9X5D2 | 3-dehydroquinate |
| CHOR_70 | | | dehydratase | glutamicum | | synthase |
| | | | | (strain ATCC | | |
| | | | | 13032/DSM | | |
| | | | | 20300/JCM | | |
| | | | | 1318/LMG | | |
| | | | | 3730/NCIMB | | |
| | | | | 10025) | | |
| CgDD | 330891.2 | Q9X5D1 | Shikimate | Corynebacterium | Q9Z470 | 3-phosphoshikimate |
| CHOR_71 | | | kinase | glutamicum | | 1-carboxyvinyl- |
| | | | | (strain ATCC | | transferase |
| | | | | 13032/DSM | | |
| | | | | 20300/JCM | | |
| | | | | 1318/LMG | | |
| | | | | 3730/NCIMB | | |
| | | | | 10025) | | |
| CgDD | 459444.8 | P32449 | Phospho-2- | Saccharomyces | B1W536 | Chorismate |
| CHOR_72 | | | dehydro-3- | cerevisiae | | dehydratase |
| | | | deoxyheptonate | (strain ATCC | | |
| | | | aldolase, | 204508/S288c) | | |
| | | | tyrosine- | (Baker's yeast) | | |
| | | | inhibited | | | |
| CgDD | 461498.3 | Q01651 | Glyceraldehyde- | Corynebacterium | B1W536 | Chorismate |
| CHOR_73 | | | 3-phosphate | glutamicum | | dehydratase |
| | | | dehydrogenase | (strain ATCC | | |
| | | | | 13032/DSM | | |
| | | | | 20300/JCM | | |
| | | | | 1318/LMG | | |
| | | | | 3730/NCIMB | | |
| | | | | 10025) | | |
| CgDD | 324549.8 | Q9X5D0 | Chorismate | Corynebacterium | Q8NQ63 | Glucose-6- |
| CHOR_75 | | | synthase | glutamicum | | phosphate 1- |
| | | | | (strain ATCC | | dehydrogenase |
| | | | | 13032/DSM | | |
| | | | | 20300/JCM | | |
| | | | | 1318/LMG | | |
| | | | | 3730/NCIMB | | |
| | | | | 10025) | | |
| CgDD | 476035.9 | B1W536 | Chorismate | Streptomyces | P32449 | Phospho-2- |
| CHOR_76 | | | dehydratase | griseus subsp. | | dehydro-3- |
| | | | | griseus (strain | | deoxyheptonate |
| | | | | JCM 4626/ | | aldolase, |
| | | | | NBRC 13350) | | tyrosine- |
| | | | | | | inhibited |
| CgDD | 323709.6 | Q9X5D0 | Chorismate | Corynebacterium | P27302 | Transketolase 1 |
| CHOR_77 | | | synthase | glutamicum | | |
| | | | | (strain ATCC | | |
| | | | | 13032/DSM | | |
| | | | | 20300/JCM | | |
| | | | | 1318/LMG | | |
| | | | | 3730/NCIMB | | |
| | | | | 10025) | | |
| CgDD | 305009.5 | Q9X5D1 | Shikimate | Corynebacterium | P15770 | Shikimate |
| CHOR_78 | | | kinase | glutamicum | | dehydrogenase |
| | | | | (strain ATCC | | |
| | | | | 13032/DSM | | |
| | | | | 20300/JCM | | |
| | | | | 1318/LMG | | |
| | | | | 3730/NCIMB | | |
| | | | | 10025) | | |
| CgDD | 325407.8 | S5V7C6 | Chorismate | Streptomyces | P27302 | Transketolase 1 |
| CHOR_79 | | | dehydratase | collinus | | |
| | | | | (strain DSM | | |
| | | | | 40733/Tu 365) | | |
| CgDD | 355969.2 | S5V7C6 | Chorismate | Streptomyces | Q8NQ65 | Transketolase |
| CHOR_80 | | | dehydratase | collinus | | |
| | | | | (strain DSM | | |
| | | | | 40733/Tu 365) | | |
| CgDD | 336678 | A0A120CSP7 | Chorismate | Streptomyces | C0ZCD4 | Chorismate |
| CHOR_81 | | | dehydratase | albus subsp. | | dehydratase |
| | | | | albus | | |
| CgDD | 333301.6 | A0A1C4UU30 | Chorismate | Micromonospora | A0A258QP84 | Chorismate |
| CHOR_82 | | | dehydratase | saelicesensis | | dehydratase |
| CgDD | 399328.2 | A0A1C4I7I3 | Chorismate | Streptomyces | A0A1G0M5U2 | Chorismate |
| CHOR_83 | | | dehydratase | sp. DvalAA-14 | | dehydratase |
| CgDD | 347447.2 | A0A117STQ9 | Chorismate | Vulcanisaeta | K1UHB8 | Chorismate |
| CHOR_84 | | | dehydratase | sp. MG_3 | | dehydratase |
| CgDD | 447918 | A0A1M5ICL3 | Chorismate | Fibrobacter | A0A285QQU7 | Chorismate |
| CHOR_85 | | | dehydratase | sp. UWB8 | | dehydratase |
| CgDD | 488724.5 | Q01651 | Glyceraldehyde- | Corynebacterium | A0A087KDJ2 | Chorismate |
| CHOR_86 | | | 3-phosphate | glutamicum | | dehydratase |
| | | | dehydrogenase | (strain ATCC | | |
| | | | | 13032/DSM | | |
| | | | | 20300/JCM | | |
| | | | | 1318/LMG | | |
| | | | | 3730/NCIMB | | |
| | | | | 10025) | | |
| CgDD | 300709.6 | Q8NPA4 | Transcriptional | Corynebacterium | Q8NNK9 | Glucose kinase |
| CHOR_87 | | | regulators | glutamicum | | |
| | | | | (strain ATCC | | |
| | | | | 13032/DSM | | |
| | | | | 20300/JCM | | |
| | | | | 1318/LMG | | |
| | | | | 3730/NCIMB | | |
| | | | | 10025) | | |
| CgDD | 336719.4 | A0A1F7LNP4 | Chorismate | Candidatus | A7H0F6 | Chorismate |
| CHOR_88 | | | dehydratase | Rokubacteria | | dehydratase |
| | | | | bacterium | | |
| | | | | GWA2_70_23 | | |
| CgDD | 445477.3 | A0A1C5BTZ0 | Chorismate | Streptomyces | A0A1Q7KZ96 | Chorismate |
| CHOR_89 | | | dehydratase | sp. MnatMP-M17 | | dehydratase |
| CgDD | 606252.3 | A0A128ATQ8 | Chorismate | Streptomyces | A0A0H3A518 | Chorismate |
| CHOR_90 | | | dehydratase | caniferus | | dehydratase |
| CgDD | 339116 | P15770 | Shikimate | Escherichia | Q9X5D1 | Shikimate |
| CHOR_91 | | | dehydrogenase | coli (strain | | kinase |
| | | | | K12) | | |
| CgDD | 317193.6 | Q9X5D0 | Chorismate | Corynebacterium | P15770 | Shikimate |
| CHOR_92 | | | synthase | glutamicum | | dehydrogenase |
| | | | | (strain ATCC | | |
| | | | | 13032/DSM | | |
| | | | | 20300/JCM | | |
| | | | | 1318/LMG | | |
| | | | | 3730/NCIMB | | |
| | | | | 10025) | | |
| CgDD | 385785.8 | A0A1G7UB78 | Chorismate | Mucilaginibacter | A0A1C6QNN8 | Chorismate |
| CHOR_93 | | | dehydratase | gossypii | | dehydratase |
| CgDD | 429442.4 | Q1Q3E4 | Chorismate | Kuenenia | Q39WZ6 | Chorismate |
| CHOR_94 | | | dehydratase | stuttgartiensis | | dehydratase |
| CgDD | 471530.6 | A0A1C0AU42 | Chorismate | Arcobacter | B0SRS6 | Chorismate |
| CHOR_96 | | | dehydratase | porcinus | | dehydratase |
| CgDD | 475908.9 | A0A100HGT8 | Chorismate | Deinococcus | D7CI10 | Chorismate |
| CHOR_97 | | | dehydratase | grandis | | dehydratase |
| CgDD | 306447.1 | A0A1M6J657 | Chorismate | Desulfotomaculum | A0A1Q8AIA2 | Chorismate |
| CHOR_98 | | | dehydratase | thermosubterraneum | | dehydratase |
| | | | | DSM 16057 | | |
| CgDD | 330681.1 | T5CLT4 | Chorismate | Helicobacter | A0A1H9WST0 | Chorismate |
| CHOR_100 | | | dehydratase | pylori FD506 | | dehydratase |
| CgDD | 304353.9 | A0A1G1LIG6 | Chorismate | Omnitrophica | A0RR58 | Chorismate |
| CHOR_101 | | | dehydratase | bacterium | | dehydratase |
| | | | | RIFCSPLOW | | |
| | | | | 02_12_FULL_50_11 | | |
| CgDD | 322992.4 | A0A1G7NYH2 | Chorismate | Pedobacterterrae | A0A1H5DL13 | Chorismate |
| CHOR_103 | | | dehydratase | | | dehydratase |
| CgDD | 557676.2 | A0A1H4B850 | Chorismate | Chitinophagaterrae | A0A1C6QNS0 | Chorismate |
| CHOR_104 | | | dehydratase | Kim and Jung 2007 | | dehydratase |
| CgDD | 484795.9 | A0A1M4VBP9 | Chorismate | Cnuella | A0A1J4U0F6 | Chorismate |
| CHOR_105 | | | dehydratase | takakiae | | dehydratase |
| CgDD | 373388.7 | A0A167DK09 | Chorismate | Paenibacillus | M3BL67 | Chorismate |
| CHOR_106 | | | dehydratase | crassostreae | | dehydratase |
| CgDD | 314410.5 | Q8NQ65 | Transketolase | Corynebacterium | P27302 | Transketolase 1 |
| CHOR_107 | | | | glutamicum | | |
| | | | | (strain ATCC | | |
| | | | | 13032/DSM | | |
| | | | | 20300/JCM | | |
| | | | | 1318/LMG | | |
| | | | | 3730/NCIMB | | |
| | | | | 10025) | | |
| CgDD | 401582.7 | A0A0B0SDC0 | Chorismate | Thermus sp. 2.9 | E1K1I4 | Chorismate |
| CHOR_108 | | | dehydratase | | | dehydratase |
| CgDD | 284953 | Q8NPA4 | Transcriptional | Corynebacterium | Q8NTX0 | Permeases of |
| CHOR_109 | | | regulators | glutamicum | | the major |
| | | | | (strain ATCC | | facilitator |
| | | | | 13032/DSM | | superfamily |
| | | | | 20300/JCM | | |
| | | | | 1318/LMG | | |
| | | | | 3730/NCIMB | | |
| | | | | 10025) | | |
| CgDD | 303982.8 | P0AC53 | Glucose-6- | Escherichia | S5V7C6 | Chorismate |
| CHOR_110 | | | phosphate 1- | coli (strain | | dehydratase |
| | | | dehydrogenase | K12) | | |
| CgDD | 586490.6 | C6J436 | Chorismate | Paenibacillus | B1W536 | Chorismate |
| CHOR_111 | | | dehydratase | sp. oral taxon | | dehydratase |
| | | | | 786 str. D14 | | |
| CgDD | 443942.3 | A0A1G9F5H7 | Chorismate | Desulfovibrio | A0A0E9AUK0 | Chorismate |
| CHOR_113 | | | dehydratase | ferrireducens | | dehydratase |
| CgDD | 562860 | Q9X5D0 | Chorismate | Corynebacterium | Q6C1X5 | Pentafunctional |
| CHOR_114 | | | synthase | glutamicum | | AROM |
| | | | | (strain ATCC | | polypeptide |
| | | | | 13032/DSM | | [Includes: 3- |
| | | | | 20300/JCM | | dehydroquinate |
| | | | | 1318/LMG | | synthase |
| | | | | 3730/NCIMB | | |
| | | | | 10025) | | |
| CgDD | 522786.1 | B1W536 | Chorismate | Streptomyces | P42850 | Phosphoenolpyruvate |
| CHOR_115 | | | dehydratase | griseus subsp. | | synthase |
| | | | | griseus (strain | | |
| | | | | JCM 4626/ | | |
| | | | | NBRC 13350) | | |
| CgDD | 328899.5 | A0A1X0Y1N7 | Chorismate | Geothermobacter | Q824C4 | Chorismate |
| CHOR_116 | | | dehydratase | sp. EPR-M | | dehydratase |
| CgDD | 306777.6 | Q2IMW4 | Multifunctional | Anaeromyxobacter | A0A0S6UB56 | Chorismate |
| CHOR_117 | | | fusion protein | dehalogenans | | dehydratase |
| | | | [Includes: Cyclic | (strain 2CP-C) | | |
| | | | dehypoxanthine | | | |
| | | | futalosine | | | |
| | | | synthase | | | |
| CgDD | 341323.4 | A0A0F0GYG6 | Chorismate | Streptomyces | A0A1Q7LJ77 | Chorismate |
| CHOR_118 | | | dehydratase | sp. NRRL F-4428 | | dehydratase |

| | | E3 | | |
|---|---|---|---|---|
| strain_name | E2 Source | Uniprot | E3 Name | E3 Source |
| CgDD | Saccharomyces | P23254 | Transketolase 1 | Saccharomyces |
| CHOR_49 | cerevisiae | | | cerevisiae |
| | (strain ATCC | | | (strain ATCC |
| | 204508/S288c) | | | 204508/S288c) |
| | (Baker's yeast) | | | (Baker's yeast) |
| CgDD | Saccharomyces | B1W536 | Chorismate | Streptomyces |
| CHOR_50 | cerevisiae | | dehydratase | griseus subsp. |
| | (strain ATCC | | | griseus (strain |
| | 204508/S288c) | | | JCM 4626/ |
| | (Baker's yeast) | | | NBRC 13350) |
| CgDD | Escherichia | A0A087KDJ2 | Chorismate | Streptomyces |
| CHOR_51 | coli (strain | | dehydratase | sp. JS01 |
| | K12) | | | |
| CgDD | Escherichia | A0A087KDJ2 | Chorismate | Streptomyces |
| CHOR_52 | coli (strain | | dehydratase | sp. JS01 |
| | K12 | | | |
| CgDD | Saccharomyces | P12008 | Chorismate | Escherichia |
| CHOR_54 | cerevisiae | | synthase | coli (strain |
| | (strain ATCC | | | K12) |
| | 204508/S288c) | | | |
| | (Baker's yeast) | | | |
| CgDD | Escherichia | A0A087KDJ2 | Chorismate | Streptomyces |
| CHOR_55 | coli (strain | | dehydratase | sp. JS01 |
| | K12) | | | |
| CgDD | Streptomyces | P27302 | Transketolase 1 | Escherichia |
| CHOR_58 | sp. JS01 | | | coli (strain |
| | | | | K12) |
| CgDD | Escherichia | P32449 | Phospho-2- | Saccharomyces |
| CHOR_59 | coli (strain | | dehydro-3- | cerevisiae |
| | K12) | | deoxyheptonate | (strain ATCC |
| | | | aldolase, | 204508/S288c) |
| | | | tyrosine- | (Baker's yeast) |
| | | | inhibited | |
| CgDD | Streptomyces | P32449 | Phospho-2- | Saccharomyces |
| CHOR_60 | griseus subsp. | | dehydro-3- | cerevisiae |
| | griseus (strain | | deoxyheptonate | (strain ATCC |
| | JCM 4626/ | | aldolase, | 204508/S288c) |
| | NBRC 13350) | | tyrosine- | (Baker's yeast) |
| | | | inhibited | |
| CgDD | Escherichia | B1W536 | Chorismate | Streptomyces |
| CHOR_61 | coli (strain | | dehydratase | griseus subsp. |
| | K12) | | | griseus (strain |
| | | | | JCM 4626/ |
| | | | | NBRC 13350) |
| CgDD | Corynebacterium | P15770 | Shikimate | Escherichia |
| CHOR_62 | glutamicum | | dehydrogenase | coli (strain |
| | (strain ATCC | | | K12) |
| | 13032/DSM | | | |
| | 20300/JCM | | | |
| | 1318/LMG | | | |
| | 3730/NCIMB | | | |
| | 10025) | | | |
| CgDD | Saccharomyces | B1W536 | Chorismate | Streptomyces |
| CHOR_64 | cerevisiae | | dehydratase | griseus subsp. |
| | (strain ATCC | | | griseus (strain |
| | 204508/S288c) | | | JCM 4626/ |
| | (Baker's yeast) | | | NBRC 13350) |
| CgDD | Thermotoga | A0A087KDJ2 | Chorismate | Streptomyces |
| CHOR_65 | maritima | | dehydratase | sp. JS01 |
| | (strain ATCC | | | |
| | 43589/MSB8/ | | | |
| | DSM 3109/ | | | |
| | JCM 10099) | | | |
| CgDD | Corynebacterium | Q9Z470 | 3-phosphoshikimate | Corynebacterium |
| CHOR_66 | glutamicum | | 1-carboxyvinyl- | glutamicum |
| | (strain ATCC | | transferase | (strain ATCC |
| | 13032/DSM | | | 13032/DSM |
| | 20300/JCM | | | 20300/JCM |
| | 1318/LMG | | | 1318/LMG |
| | 3730/NCIMB | | | 3730/NCIMB |
| | 10025) | | | 10025) |
| CgDD | Corynebacterium | Q9X5D2 | 3-dehydroquinate | Corynebacterium |
| CHOR_67 | glutamicum | | synthase | glutamicum |
| | (strain ATCC | | | (strain ATCC |
| | 13032/DSM | | | 13032/DSM |
| | 20300/JCM | | | 20300/JCM |
| | 1318/LMG | | | 1318/LMG |
| | 3730/NCIMB | | | 3730/NCIMB |
| | 10025) | | | 10025) |
| CgDD | Corynebacterium | Q9Z470 | 3-phosphoshikimate | Corynebacterium |
| CHOR_68 | glutamicum | | 1-carboxyvinyl- | glutamicum |
| | (strain ATCC | | transferase | (strain ATCC |
| | 13032/DSM | | | 13032/DSM |
| | 20300/JCM | | | 20300/JCM |
| | 1318/LMG | | | 1318/LMG |
| | 3730/NCIMB | | | 3730/NCIMB |
| | 10025) | | | 10025) |
| CgDD | Corynebacterium | Q9X5D0 | Chorismate | Corynebacterium |
| CHOR_69 | glutamicum | | synthase | glutamicum |
| | (strain ATCC | | | (strain ATCC |
| | 13032/DSM | | | 13032/DSM |
| | 20300/JCM | | | 20300/JCM |
| | 1318/LMG | | | 1318/LMG |
| | 3730/NCIMB | | | 3730/NCIMB |
| | 10025) | | | 10025) |
| CgDD | Corynebacterium | P15770 | Shikimate | Escherichia |
| CHOR_70 | glutamicum | | dehydrogenase | coli (strain |
| | (strain ATCC | | | K12) |
| | 13032/DSM | | | |
| | 20300/JCM | | | |
| | 1318/LMG | | | |
| | 3730/NCIMB | | | |
| | 10025) | | | |
| CgDD | Corynebacterium | Q9X5D0 | Chorismate | Corynebacterium |
| CHOR_71 | glutamicum | | synthase | glutamicum |
| | (strain ATCC | | | (strain ATCC |
| | 13032/DSM | | | 13032/DSM |
| | 20300/JCM | | | 20300/JCM |
| | 1318/LMG | | | 1318/LMG |
| | 3730/NCIMB | | | 3730/NCIMB |
| | 10025) | | | 10025) |
| CgDD | Streptomyces | Q8NQ65 | Transketolase | Corynebacterium |
| CHOR_72 | griseus subsp. | | | glutamicum |
| | griseus (strain | | | (strain ATCC |
| | JCM 4626/ | | | 13032/DSM |
| | NBRC 13350) | | | 20300/JCM |
| | | | | 1318/LMG |
| | | | | 3730/NCIMB |
| | | | | 10025) |
| CgDD | Streptomyces | Q8NQ65 | Transketolase | Corynebacterium |
| CHOR_73 | griseus subsp. | | | glutamicum |
| | griseus (strain | | | (strain ATCC |
| | JCM 4626/ | | | 13032/DSM |
| | NBRC 13350) | | | 20300/JCM |
| | | | | 1318/LMG |
| | | | | 3730/NCIMB |
| | | | | 10025) |
| CgDD | Corynebacterium | P08566 | Pentafunctional | Saccharomyces |
| CHOR_75 | glutamicum | | AROM | cerevisiae |
| | (strain ATCC | | polypeptide | (strain ATCC |
| | 13032/DSM | | [Includes: 3- | 204508/S288c) |
| | 20300/JCM | | dehydroquinate | (Baker's yeast) |
| | 1318/LMG | | synthase | |
| | 3730/NCIMB | | | |
| | 10025) | | | |
| CgDD | Saccharomyces | P08566 | Pentafunctional | Saccharomyces |
| CHOR_76 | cerevisiae | | AROM | cerevisiae |
| | (strain ATCC | | polypeptide | (strain ATCC |
| | 204508/S288c) | | [Includes: 3- | 204508/S288c) |
| | (Baker's yeast) | | dehydroquinate | (Baker's yeast) |
| | | | synthase | |
| CgDD | Escherichia | P08566 | Pentafunctional | Saccharomyces |
| CHOR_77 | coli (strain | | AROM | cerevisiae |
| | K12) | | polypeptide | (strain ATCC |
| | | | [Includes: 3- | 204508/S288c) |
| | | | dehydroquinate | (Baker's yeast) |
| | | | synthase | |
| CgDD | Escherichia | O52377 | 3-dehydroquinate | Corynebacterium |
| CHOR_78 | coli (strain | | dehydratase | glutamicum |
| | K12) | | | (strain ATCC |
| | | | | 13032/DSM |
| | | | | 20300/JCM |
| | | | | 1318/LMG |
| | | | | 3730/NCIMB |
| | | | | 10025) |
| CgDD | Escherichia | P23538 | Phosphoenolpyruvate | Escherichia |
| CHOR_79 | coli (strain | | synthase | coli (strain |
| | K12) | | | K12) |
| CgDD | Corynebacterium | Q8NQ63 | Glucose-6- | Corynebacterium |
| CHOR_80 | glutamicum | | phosphate 1- | glutamicum |
| | (strain ATCC | | dehydrogenase | (strain ATCC |
| | 13032/DSM | | | 13032/DSM |
| | 20300/JCM | | | 20300/JCM |
| | 1318/LMG | | | 1318/LMG |
| | 3730/NCIMB | | | 3730/NCIMB |
| | 10025) | | | 10025) |
| CgDD | Brevibacillus | C3XKR6 | Chorismate | Helicobacter |
| CHOR_81 | brevis (strain | | dehydratase | winghamensis |
| | 47/JCM 6285/ | | | ATCC BAA-430 |
| | NBRC 100599) | | | |
| CgDD | Sulfurovum | A0A1H6GTJ5 | Chorismate | Selenomonas |
| CHOR_82 | sp. 28-43-6 | | dehydratase | ruminantium |
| CgDD | Geobacteraceae | D4S428 | Chorismate | Selenomonasnoxia |
| CHOR_83 | bacterium | | dehydratase | ATCC 43541 |
| | GWC2_58_44 | | | |
| CgDD | Streptomyces | A0A090ZFP6 | Chorismate | Paenibacillus |
| CHOR_84 | sp. SM8 | | dehydratase | macerans |
| | | | | (Bacillus |
| | | | | macerans) |
| CgDD | Streptomyces | A0A0N0YVQ2 | Chorismate | Streptomyces |
| CHOR_85 | sp. 1331.2 | | dehydratase | sp. NRRL F-6602 |
| CgDD | Streptomyces | P0A6E1 | Shikimate kinase 2 | Escherichia |
| CHOR_86 | sp. JS01 | | | coli (strain |
| | | | | K12) |
| CgDD | Corynebacterium | Q8NTX0 | Permeases of | Corynebacterium |
| CHOR_87 | glutamicum | | the major | glutamicum |
| | (strain ATCC | | facilitator | (strain ATCC |
| | 13032/DSM | | superfamily | 13032/DSM |
| | 20300/JCM | | | 20300/JCM |
| | 1318/LMG | | | 1318/LMG |
| | 3730/NCIMB | | | 3730/NCIMB |
| | 10025) | | | 10025) |
| CgDD | Campylobacter | A0A099TG89 | Chorismate | Helicobacter |
| CHOR_88 | curvus (strain | | dehydratase | sp. MIT 05-5293 |
| | 525.92) | | | |
| CgDD | Acidobacteria | A0A1Q5X8M2 | Chorismate | Paenibacillus |
| CHOR_89 | bacterium | | dehydratase | sp. P3E |
| | 13_1_40CM_4_58_4 | | | |
| CgDD | Desulfovibrio | C6CUC4 | Chorismate | Paenibacillus |
| CHOR_90 | vulgaris subsp. | | dehydratase | sp. (strain |
| | vulgaris | | | JDR-2) |
| | (strain DP4) | | | |
| CgDD | Corynebacterium | Q9Z470 | 3-phosphoshikimate | Corynebacterium |
| CHOR_91 | glutamicum | | 1-carboxyvinyl | glutamicum |
| | (strain ATCC | | transferase | (strain ATCC |
| | 13032/DSM | | | 13032/DSM |
| | 20300/JCM | | | 20300/JCM |
| | 1318/LMG | | | 1318/LMG |
| | 3730/NCIMB | | | 3730/NCIMB |
| | 10025) | | | 10025) |
| CgDD | Escherichia | Q9X5D1 | Shikimate | Corynebacterium |
| CHOR_92 | coli (strain | | kinase | glutamicum |
| | K12) | | | (strain ATCC |
| | | | | 13032/DSM |
| | | | | 20300/JCM |
| | | | | 1318/LMG |
| | | | | 3730/NCIMB |
| | | | | 10025) |
| CgDD | Streptomyces | A0A1E3XBU7 | Chorismate | Candidatus |
| CHOR_93 | sp. AmelKG-E11A | | dehydratase | Scalindua |
| | | | | rubra |
| CgDD | Geobacter | HOBDI5 | Chorismate | Streptomyces |
| CHOR_94 | metallireducens | | dehydratase | sp. W007 |
| | (strain GS-15/ | | | |
| | ATCC 53774/ | | | |
| | DSM 7210) | | | |
| CgDD | Leptospira | C9YXX4 | Chorismate | Streptomyces |
| CHOR_96 | biflexa | | dehydratase | scabiei (strain |
| | serovar Patoc | | | 87.22) |
| | (strain Patoc | | | |
| | 1/ATCC 23582/ | | | |
| | Paris) | | | |
| CgDD | Streptomyces | A4J6K6 | Chorismate | Desulfotomaculum |
| CHOR_97 | bingchenggensis | | dehydratase | reducens |
| | (strain BCW-1) | | | (strain MI-1) |
| CgDD | Acidobacteria | A0A1V3AJD3 | Chorismate | Helicobacter |
| CHOR_98 | bacterium | | dehydratase | pylori |
| | 13_1_20CM_2_65_9 | | | (Campylobacter |
| | | | | pylori) |
| CgDD | Streptomyces | A0A1F3LRF7 | Chorismate | Bacteroidetes |
| CHOR_100 | qinglanensis | | dehydratase | bacterium |
| | | | | GWF2_40_14 |
| CgDD | Campylobacter | E4MFL0 | Chorismate | Alistipes sp. |
| CHOR_101 | fetus subsp. | | dehydratase | HGB5 |
| | fetus (strain | | | |
| | 82-40) | | | |
| CgDD | Streptomyces | A0A1G8H2G6 | Chorismate | Aneurinibacillus |
| CHOR_103 | sp. 3213 | | dehydratase | migulanus |
| | | | | (Bacillus |
| | | | | migulanus) |
| CgDD | Streptomyces | H6QD16 | Chorismate | Pyrobaculum |
| CHOR_104 | | | dehydratase | oguniense |
| | | | | (strain DSM |
| | | | | 13380/JCM |
| | | | | 10595/TE7) |
| CgDD | Helicobacteraceae | A0A0A8H3E8 | Chorismate | Campylobacter |
| CHOR_105 | bacterium | | dehydratase | insulaenigrae |
| | CG1_02_36_14 | | | NCTC 12927 |
| CgDD | Streptomyces | A0A0K2Y5W0 | Chorismate | Helicobacter |
| CHOR_106 | mobaraensis | | dehydratase | heilmannii |
| | NBRC 13819 = | | | |
| | DSM 40847 | | | |
| CgDD | Escherichia | P23254 | Transketolase 1 | Saccharomyces |
| CHOR_107 | coli (strain | | | cerevisiae |
| | K12) | | | (strain ATCC |
| | | | | 204508/S288c) |
| | | | | (Baker's yeast) |
| CgDD | Desulfovibrio | A0A1C5E503 | Chorismate | Streptomyces |
| CHOR_108 | fructosivorans | | dehydratase | sp. DconLS |
| | JJ | | | |
| CgDD | Corynebacterium | Q8NNK9 | Glucose | Corynebacterium |
| CHOR_109 | glutamicum | | kinase | glutamicum |
| | (strain ATCC | | | (strain ATCC |
| | 13032/DSM | | | 13032/DSM |
| | 20300/JCM | | | 20300/JCM |
| | 1318/LMG | | | 1318/LMG |
| | 3730/NCIMB | | | 3730/NCIMB |
| | 10025) | | | 10025) |
| CgDD | Streptomyces | Q9X5D0 | Chorismate | Corynebacterium |
| CHOR_110 | collinus | | synthase | glutamicum |
| | (strain DSM | | | (strain ATCC |
| | 40733/Tu 365) | | | 13032/DSM |
| | | | | 20300/JCM |
| | | | | 1318/LMG |
| | | | | 3730/NCIMB |
| | | | | 10025) |
| CgDD | Streptomyces | S5V7C6 | Chorismate | Streptomyces |
| CHOR_111 | griseus subsp. | | dehydratase | collinus |
| | griseus (strain | | | (strain DSM |
| | JCM 4626/ | | | 40733/Tu 365) |
| | NBRC 13350) | | | |
| CgDD | Chlamydia | A0A1V2ECE5 | Chorismate | Leptospira |
| CHOR_113 | trachomatis | | dehydratase | santarosai |
| | | | | serovar |
| | | | | Grippotyphosa |
| CgDD | Yarrowia | A0A087KDJ2 | Chorismate | Streptomyces |
| CHOR_114 | lipolytica | | dehydratase | sp. JS01 |
| | (strain CLIB | | | |
| | 122/E 150) | | | |
| | (Yeast) | | | |
| | (Candida | | | |
| | lipolytica) | | | |
| CgDD | Pyrococcus | Q8NQ65 | Transketolase | Corynebacterium |
| CHOR_115 | furiosus | | | glutamicum |
| | (strain ATCC | | | (strain ATCC |
| | 43587/DSM | | | 13032/DSM |
| | 3638/JCM | | | 20300/JCM |
| | 8422/Vc1) | | | 1318/LMG |
| | | | | 3730/NCIMB |
| | | | | 10025) |
| CgDD | Chlamydophila | A0A1X8WQP2 | Chorismate | Leptospira |
| CHOR_116 | caviae | | dehydratase | interrogans |
| | (strain GPIC) | | | serovar |
| | | | | Canicola |
| CgDD | Moorella | K1UHB8 | Chorismate | Streptomyces |
| CHOR_117 | thermoacetica | | dehydratase | sp. SM8 |
| | Y72 | | | |
| CgDD | Gemmatimonadetes | A0A1W2E653 | Chorismate | Sporomusa |
| CHOR_118 | bacterium | | dehydratase | malonica |
| | 13_1_40CM_3_65_8 | | | |

TABLE 5
Fifth-Round Results
In addition to the enzymes in this table, the Corynebacterium glutamicum strains contain
two copies of chorismate dehydratase from Streptomyces griseus (UniProt ID
B1W536), a feedback-deregulated variant of an Escherichia coli K12 DAHP synthase
(UniProt ID POAB91) including P150L, and three further chorismate dehydratases: one
from Streptomyces caniferus (UniProt ID A0A128ATQ8), one from Disulfovibrio vulgaris
(Uniprot ID A0AOH3A518), and one from Paenibacillus sp. (strain JDR-2) (UniProt ID C6CUC4),

| strain | Titer | E1 | | | E2 |
|---|---|---|---|---|---|
| name | μg/L | Uniprot | E1 Name | E1 Source | Uniprot |
| CgDD | 651.71605 | Q6C1X5 | Pentafunctional AROM | Yarrowia | A0A087KDJ2 |
| CHOR_122 | | | polypeptide [Includes: | lipolytica (strain | |
| | | | 3-dehydroquinate | CLIB 122/E 150) | |
| | | | synthase | (Yeast) (Candida | |
| | | | | lipolytica) | |
| CgDD | 667.2626 | P42850 | Phosphoenolpyruvate | Pyrococcus | B1W536 |
| CHOR_123 | | | synthase | furiosus (strain | |
| | | | | ATCC 43587/ | |
| | | | | DSM 3638/JCM | |
| | | | | 8422/Vc1) | |
| CgDD | 603.674575 | Q9S6G5 | 3-dehydroquinate | Corynebacterium | |
| CHOR_144 | | | dehydratase | glutamicum | |
| | | | | (Brevibacterium | |
| | | | | saccharolyticum) | |
| CgDD | 588.651225 | A4QEJ8 | Chorismate synthase | Corynebacterium | |
| CHOR_158 | | | | glutamicum | |
| | | | | (strain R) | |
| CgDD | 370.7984 | P0A870 | Transaldolase B | Escherichia coli | |
| CHOR_163 | | | | (strain K12) | |
| CgDD | 578.26485 | Q9X5D1 | Shikimate kinase | Corynebacterium | |
| CHOR_164 | | | | glutamicum | |
| | | | | (strain ATCC | |
| | | | | 13032/DSM | |
| | | | | 20300/JCM | |
| | | | | 1318/LMG 3730/ | |
| | | | | NCIMB 10025) | |
| CgDD | 478.041525 | A3PMF8 | Aminotransferase | Rhodobacter | Q9X5D0 |
| CHOR_170 | | | | sphaeroides | |
| | | | | (strain ATCC | |
| | | | | 17029/ATH 2.4.9) | |
| CgDD | 490.4377 | P12008 | Chorismate synthase | Escherichia coli | |
| CHOR_171 | | | | (strain K12) | |
| CgDD | 403.9325333 | P10880 | Shikimate kinase 2 | Dickeya | |
| CHOR_172 | | | | chrysanthemi | |
| | | | | (Pectobacterium | |
| | | | | chrysanthemi) | |
| | | | | (Erwinia | |
| | | | | chrysanthemi) | |
| CgDD | 611.2488 | P05194 | 3-dehydroquinate | Escherichia coli | |
| CHOR_178 | | | dehydratase | (strain K12) | |
| CgDD | 391.088 | Q6C5J7 | YALIOE17479p | Yarrowia | |
| CHOR_179 | | | | lipolytica (strain | |
| | | | | CLIB 122/E 150) | |
| | | | | (Yeast) (Candida | |
| | | | | lipolytica) | |
| CgDD | 606.55835 | A0A0A8H3E8 | Chorismate | Campylobacter | A0A1J4U0F6 |
| CHOR_127 | | | dehydratase | insulaenigrae | |
| | | | | NCTC 12927 | |
| CgDD | 810.035325 | A0A0H3A518 | Chorismate | Desulfovibrio | C6CUC4 |
| CHOR_128 | | | dehydratase | vulgaris subsp. | |
| | | | | vulgaris (strain | |
| | | | | DP4) | |
| CgDD | 0 | A4QC99 | 3-phosphoshikimate 1- | Corynebacterium | |
| CHOR_132 | | | carboxyvinyltransferas | glutamicum | |
| | | | e | (strain R) | |
| CgDD | 594.3823 | Q9X5C9 | Quinate/shikimate | Corynebacterium | |
| CHOR_134 | | | dehydrogenase | glutamicum | |
| | | | | (strain ATCC | |
| | | | | 13032/DSM | |
| | | | | 20300/JCM | |
| | | | | 1318/LMG 3730/ | |
| | | | | NCIMB 10025) | |
| CgDD | 583.909325 | P0A6E1 | Shikimate kinase 2 | Escherichia coli | |
| CHOR_138 | | | | (strain K12) | |
| CgDD | 534.962225 | Q9X5D1 | Shikimate kinase | Corynebacterium | |
| CHOR_139 | | | | glutamicum | |
| | | | | (strain ATCC | |
| | | | | 13032/DSM | |
| | | | | 20300/JCM | |
| | | | | 1318/LMG 3730/ | |
| | | | | NCIMB 10025) | |
| CgDD | 586.89615 | Q02635 | Aspartate | Rhizobium | |
| CHOR_147 | | | aminotransferase A | meliloti (strain | |
| | | | | 1021) (Ensifer | |
| | | | | meliloti) | |
| | | | | (Sinorhizobium | |
| | | | | meliloti) | |
| CgDD | 490.90655 | P0AC53 | Glucose-6-phosphate | Escherichia coli | |
| CHOR_153 | | | 1-dehydrogenase | (strain K12) | |
| CgDD | 0 | Q9X5D0 | Chorismate synthase | Corynebacterium | |
| CHOR_174 | | | | glutamicum | |
| | | | | (strain ATCC | |
| | | | | 13032/DSM | |
| | | | | 20300/JCM | |
| | | | | 1318/LMG 3730/ | |
| | | | | NCIMB 10025) | |
| CgDD | 582.168325 | P0A6E1 | Shikimate kinase 2 | Escherichia coli | |
| CHOR_176 | | | | (strain K 12) | |
| CgDD | 523.33195 | A3PMF8 | Aminotransferase | Rhodobacter | |
| CHOR_183 | | | | sphaeroides | |
| | | | | (strain ATCC | |
| | | | | 17029/ATH | |
| | | | | 2.4.9) | |
| CgDD | 532.946825 | Q01651 | Glyceraldehyde-3- | Corynebacterium | POA6E1 |
| CHOR_157 | | | phosphate | glutamicum | |
| | | | dehydrogenase | (strain ATCC | |
| | | | | 13032/DSM | |
| | | | | 20300/JCM | |
| | | | | 1318/LMG 3730/ | |
| | | | | NCIMB 10025) | |
| CgDD | 556.6101 | Q9X5D2 | 3-dehydroquinate | Corynebacterium | |
| CHOR_121 | | | synthase | glutamicum | |
| | | | | (strain ATCC | |
| | | | | 13032/DSM | |
| | | | | 20300/JCM | |
| | | | | 1318/LMG 3730/ | |
| | | | | NCIMB 10025) | |
| CgDD | 559.4479 | A0A1H4B850 | Chorismate | Chitinophagaterrae | H6QD16 |
| CHOR_125 | | | dehydratase | Kim and | |
| | | | | Jung 2007 | |
| CgDD | 573.248825 | Q82WA8 | Aminotransferase | Nitrosomonas | |
| CHOR_130 | | | | europaea (strain | |
| | | | | ATCC 19718/ | |
| | | | | CIP 103999/ | |
| | | | | KCTC 2705/ | |
| | | | | NBRC 14298) | |
| CgDD | 569.5876 | P73906 | Prephenate | Synechocystis | |
| CHOR_150 | | | dehydrogenase | sp. (strain PCC | |
| | | | | 6803/Kazusa) | |
| CgDD | 422.69145 | P28777 | Chorismate synthase | Saccharomyces | |
| CHOR_175 | | | | cerevisiae (strain | |
| | | | | ATCC 204508/ | |
| | | | | S288c) (Baker's | |
| | | | | yeast) | |
| CgDD | 530.51805 | P0A6D3 | 3-phosphoshikimate 1- | Escherichia coli | |
| CHOR_181 | | | carboxyvinyltransferase | (strain K12) | |
| CgDD | 565.458525 | Q9X5C9 | Quinate/shikimate | Corynebacterium | |
| CHOR_182 | | | dehydrogenase | glutamicum | |
| | | | | (strain ATCC | |
| | | | | 13032/DSM | |
| | | | | 20300/JCM | |
| | | | | 1318/LMG 3730/ | |
| | | | | NCIMB 10025) | |
| CgDD | 636.247275 | P35170 | Phospho-2-dehydro- | Corynebacterium | |
| CHOR_116 | | | 3-deoxyheptonate | glutamicum | |
| | | | aldolase | (strain ATCC | |
| | | | | 13032/DSM | |
| | | | | 20300/JCM | |
| | | | | 1318/LMG 3730/ | |
| | | | | NCIMB 10025) | |
| CgDD | 551.1595 | P35170 | Phospho-2-dehydro- | Corynebacterium | |
| CHOR_119 | | | 3-deoxyheptonate | glutamicum | |
| | | | aldolase | (strain ATCC | |
| | | | | 13032/DSM | |
| | | | | 20300/JCM | |
| | | | | 1318/LMG 3730/ | |
| | | | | NCIMB 10025) | |
| CgDD | 431.556875 | P10880 | Shikimate kinase 2 | Dickeya | |
| CHOR_154 | | | | chrysanthemi | |
| | | | | (Pectobacterium | |
| | | | | chrysanthemi) | |
| | | | | (Erwinia | |
| | | | | chrysanthemi) | |
| CgDD | 290.6759 | P27302 | Transketolase 1 | Escherichia coli | |
| CHOR_156 | | | | (strain K12) | |
| CgDD | 571.8747 | P12008 | Chorismate synthase | Escherichia coli | |
| CHOR_162 | | | | (strain K12) | |
| CgDD | 579.875475 | P56073 | Shikimate kinase | Helicobacter | |
| CHOR_168 | | | | pylori (strain | |
| | | | | ATCC 700392/ | |
| | | | | 26695) | |
| | | | | (Campylobacter | |
| | | | | pylori) | |
| CgDD | 221.1131667 | P52987 | Glyceraldehyde-3- | Lactococcus | |
| CHOR_180 | | | phosphate | lactis subsp. | |
| | | | dehydrogenase | lactis (strain | |
| | | | | IL 1403) | |
| | | | | (Streptococcus | |
| | | | | lactis) | |
| CgDD | 558.384775 | P0A870 | Transaldolase B | Escherichia coli | |
| CHOR_184 | | | | (strain K12) | |

| strain | | | E3 | | |
|---|---|---|---|---|---|
| name | E2 Name | E2 Source | Uniprot | E3 Name | E3 Source |
| CgDD | Chorismate | Streptomyces | Q9X5D0 | Chorismate | Corynebacterium |
| CHOR_122 | dehydratase | sp. JS01 | | synthase | glutamicum |
| | | | | | (strain ATCC |
| | | | | | 13032/DSM |
| | | | | | 20300/JCM |
| | | | | | 1318/LMG 3730/ |
| | | | | | NCIMB 10025) |
| CgDD | Chorismate | Streptomyces | Q8NQ65 | Transketolase | Corynebacterium |
| CHOR_123 | dehydratase | griseus subsp. | | | glutamicum |
| | | griseus (strain | | | (strain ATCC |
| | | JCM 4626/ | | | 13032/DSM |
| | | NBRC 13350) | | | 20300/JCM |
| | | | | | 1318/LMG 3730/ |
| | | | | | NCIMB 10025) |
| CgDD | | | | | |
| CHOR_144 | | | | | |
| CgDD | | | | | |
| CHOR_158 | | | | | |
| CgDD | | | | | |
| CHOR_163 | | | | | |
| CgDD | | | | | |
| CHOR_164 | | | | | |
| CgDD | Chorismate | Corynebacterium | P73906 | Prephenate | Synechocystis sp. |
| CHOR_170 | synthase | glutamicum | | dehydrogenase | (strain PCC 6803/ |
| | | (strain ATCC | | | Kazusa) |
| | | 13032/DSM | | | |
| | | 20300/JCM | | | |
| | | 1318/LMG | | | |
| | | 3730/NCIMB | | | |
| | | 10025) | | | |
| CgDD | | | | | |
| CHOR_171 | | | | | |
| CgDD | | | | | |
| CHOR_172 | | | | | |
| CgDD | | | | | |
| CHOR_178 | | | | | |
| CgDD | | | | | |
| CHOR_179 | | | | | |
| CgDD | Chorismate | Helicobacteraceae | A0A1M4VBP9 | Chorismate | Cnuella takakiae |
| CHOR_127 | dehydratase | bacterium | | dehydratase | |
| | | CG1_02_36_14 | | | |
| CgDD | Chorismate | Paenibacillus | A0A128ATQ8 | Chorismate | Streptomyces |
| CHOR_128 | dehydratase | sp. (strain | | dehydratase | caniferus |
| | | JDR-2) | | | |
| CgDD | | | | | |
| CHOR_132 | | | | | |
| CgDD | | | | | |
| CHOR_134 | | | | | |
| CgDD | | | | | |
| CHOR_138 | | | | | |
| CgDD | | | | | |
| CHOR_139 | | | | | |
| CgDD | | | | | |
| CHOR_147 | | | | | |
| CgDD | | | | | |
| CHOR_153 | | | | | |
| CgDD | | | | | |
| CHOR_174 | | | | | |
| CgDD | | | | | |
| CHOR_176 | | | | | |
| CgDD | | | | | |
| CHOR_183 | | | | | |
| CgDD | Shikimate | Escherichia | A0A087KDJ2 | Chorismate | Streptomycessp. |
| CHOR_157 | kinase 2 | coli (strain | | dehydratase | JS01 |
| | | K12) | | | |
| CgDD | | | | | |
| CHOR_121 | | | | | |
| CgDD | Chorismate | Pyrobaculum | A0A1C6QNS0 | Chorismate | Streptomyces |
| CHOR_125 | dehydratase | oguniense | | dehydratase | |
| | | (strain DSM | | | |
| | | 13380/JCM | | | |
| | | 10595/TE7) | | | |
| CgDD | | | | | |
| CHOR_130 | | | | | |
| CgDD | | | | | |
| CHOR_150 | | | | | |
| CgDD | | | | | |
| CHOR_175 | | | | | |
| CgDD | | | | | |
| CHOR_181 | | | | | |
| CgDD | | | | | |
| CHOR_182 | | | | | |
| CgDD | | | | | |
| CHOR_116 | | | | | |
| CgDD | | | | | |
| CHOR_119 | | | | | |
| CgDD | | | | | |
| CHOR_154 | | | | | |
| CgDD | | | | | |
| CHOR_156 | | | | | |
| CgDD | | | | | |
| CHOR_162 | | | | | |
| CgDD | | | | | |
| CHOR_168 | | | | | |
| CgDD | | | | | |
| CHOR_180 | | | | | |
| CgDD | | | | | |
| CHOR_184 | | | | | |

### REFERENCES

1. Wei. T., B. Y. Cheng, and J. Z. Liu, Genome engineering Escherichia coli for L-DOPA overproduction from glucose. Sci Rep. 2016. 6: p. 30080.
2. Parche, S., et al., Corynebacterium glutamicum: a dissection of the PTS. J Mol Microbiol Biotechnol, 2001. 3(3): p. 423-8.

What is claimed is:

1. An engineered microbial cell that expresses a non-native chorismate dehydratase, wherein the engineered microbial cell produces deoxyhydrochorismic acid, optionally wherein, when cultured, the engineered microbial cell produces deoxyhydrochorismic acid at a level of at least 20, 50, 100, 500, 1000, or 1500 mg/L of culture medium.

2. The engineered microbial cell of claim 1, wherein the engineered microbial cell comprises increased activity of one or more upstream deoxyhydrochorismic acid pathway enzyme(s), said increased activity being increased relative to a control cell, optionally wherein the one or more upstream deoxyhydrochorismic acid pathway enzyme(s) are selected from the group consisting of a glucokinase, a transketolase, a transaldolase, phospho-2-dehydro-3-deoxyheptonate aldolase, a 3-deoxy-D-arabino-heptulosonate-7-phosphate (DAHP) synthase, a 3-dehydroquinate synthase, a 3-dehydroquinate dehydratase, a shikimate dehydrogenase, a shikimate kinase, a 3-phosphoshikimate 1-carboxyvinyltransferase, and a chorismate synthase.

3. The engineered microbial cell of claim 1 or claim 2, wherein the engineered microbial cell comprises reduced activity of one or more enzyme(s) that consume one or more deoxyhydrochorismic acid pathway precursors, said reduced activity being reduced relative to a control cell, optionally wherein the one or more enzyme(s) that consume one or more deoxyhydrochorismic acid pathway precursors are selected from the group consisting of dihydroxyacetone phosphatase and phosphoenolpyruvate phosphotransferase.

4. The engineered microbial cell of any one of claims 1-3, wherein the engineered microbial cell additionally expresses a feedback-deregulated DAHP synthase.

5. The engineered microbial cell of any one of claims 1-4, wherein the engineered microbial cell comprises increased activity of one or more enzyme(s) that increase the supply of the reduced form of nicotinamide adenine dinucleotide phosphate (NADPH), said increased activity being increased relative to a control cell, optionally wherein the one or more enzyme(s) that increase the supply of the reduced form of NADPH are selected from the group consisting of pentose phosphate pathway enzymes, NADP+-dependent glyceraldehyde 3-phosphate dehydrogenase (GAPDH), and NADP+-dependent glutamate dehydrogenase.

6. The engineered microbial cell of any one of claims 1-5, wherein the engineered microbial cell comprises a Saccharomyces cerevisiae cell.

7. The engineered microbial cell of any one of claims 1-6, wherein the non-native chorismate dehydratase comprises a chorismate dehydratase having at least 70% amino acid sequence identity with a chorismate dehydratase from an organism selected from the group consisting of Paenibacillus sp. oral taxon 786 str. D14, Paenibacillus sp. (strain JDR-2), and Pedobacter heparinus, wherein:
the chorismate dehydratase from Paenibacillus sp. oral taxon 786 str. D14 comprises SEQ ID NO:1;
the chorismate dehydratase from Paenibacillus sp. (strain JDR-2) comprises SEQ ID NO:2; and
the chorismate dehydratase from Pedobacter heparinus comprises SEQ ID NO:3.

8. The engineered microbial cell of claim 7, wherein the non-native chorismate dehydratase comprises a chorismate dehydratase having at least 70% amino acid sequence identity with the chorismate dehydratase from Paenibacillus sp. oral taxon 786 str. D14.

9. The engineered microbial cell of any one of claims 1 and 6-8, wherein the engineered microbial cell comprises increased activity of one or more upstream deoxyhydrochorismic acid pathway enzyme(s), said increased activity being increased relative to a control cell, wherein the one or more upstream deoxyhydrochorismic acid pathway enzyme(s) comprise a dehydroquinate synthase or a shikimate kinase.

10. The engineered microbial cell of claim 9, wherein the heterologous dehydroquinate synthase has at least 70% amino acid sequence identity with a dehydroquinate synthase from Corynebacterium glutamicum comprising SEQ ID NO:4.

11. The engineered microbial cell of claim 10, wherein the heterologous shikimate kinase has at least 70% amino acid sequence identity with a shikimate kinase from Corynebacterium glutamicum comprising SEQ ID NO:5.

12. The engineered microbial cell of claim 11, wherein the engineered microbial cell expresses an additional copy of a chorismate dehydratase having at least 70% amino acid sequence identity with the chorismate dehydratase from Paenibacillus sp. (strain JDR-2) or Pedobacter heparinus.

13. The engineered microbial cell of any one of claims 4 and 6-12, wherein the feedback-deregulated DAHP synthase is a feedback-deregulated variant of a S. cerevisiae DAHP synthase that comprises amino acid substitution K229L and has at least 70% amino acid sequence identity with SEQ ID NO: 6.

14. The engineered microbial cell of any one of claims 1-6, wherein the engineered microbial cell is a Corynebacterium glutamicum cell.

15. The engineered microbial cell of claim 14, wherein the non-native chorismate dehydratase comprises a chorismate dehydratase having at least 70% amino acid sequence identity with a chorismate dehydratase from an organism selected from the group consisting of Streptomyces griseus, Streptomyces coelicolor, Streptomyces sp Mg1, Streptomyces collinus, Salinispora arenicola, Streptomyces leeuwenhoekii, Leptospira mayottensis, and Paenibacillus sp. (strain JDR-2), wherein:
the chorismate dehydratase from Streptomyces griseus comprises SEQ ID NO:7;
the chorismate dehydratase from Streptomyces coelicolor comprises SEQ ID NO:8;
the chorismate dehydratase from Streptomyces sp Mg1 comprises SEQ ID NO:9;
the chorismate dehydratase from Streptomyces collinus comprises SEQ ID NO:10;
the chorismate dehydratase from Salinispora arenicola comprises SEQ ID NO:11;
the chorismate dehydratase from Streptomyces leeuwenhoekii comprises SEQ ID NO: 12;
the chorismate dehydratase from Leptospira mayottensis comprises SEQ ID NO:13; and
the chorismate dehydratase from Paenibacillus sp. (strain JDR-2) comprises SEQ ID NO:2.

16. The engineered microbial cell of claim 15, wherein the non-native chorismate dehydratase comprises a chorismate dehydratase having at least 70% amino acid sequence identity with a chorismate dehydratase from Streptomyces griseus comprising SEQ ID NO:7.

17. The engineered microbial cell of any one of claims 4 and 14-16,
wherein the feedback-deregulated DAHP synthase is a feedback-deregulated variant of an Escherichia coli K12 DAHP synthase that comprises amino acid substitution P150L and has at least 70% amino acid sequence identity with SEQ ID NO:15.

18. The engineered microbial cell of claim 17, wherein the engineered microbial cell additionally expresses:
a chorismate dehydratase having at least 70% amino acid sequence identity with a chorismate dehydratase from Strepomyces caniferus comprising SEQ ID NO:16;
a chorismate dehydratase having at least 70% amino acid sequence identity with a chorismate dehydratase from Desulfovibrio vulgaris subsp. vulgaris (strain DP4) comprising SEQ ID NO:17 and
a chorismate dehydratase having at least 70% amino acid sequence identity with a chorismate dehydratase from Paenibacillus sp. (strain JDR-2) comprising SEQ ID NO:2.

19. The engineered microbial cell of claim 18, wherein the engineered microbial cell expresses at least two copies each of:
a chorismate dehydratase having at least 70% amino acid sequence identity with a chorismate dehydratase from Strepomyces caniferus comprising SEQ ID NO:16;
a chorismate dehydratase having at least 70% amino acid sequence identity with a chorismate dehydratase from Desulfovibrio vulgaris subsp. vulgaris (strain DP4) comprising SEQ ID NO: 17; and
a chorismate dehydratase having at least 70% amino acid sequence identity with a chorismate dehydratase from Paenibacillus sp. (strain JDR-2) comprising SEQ ID NO:2.
